(19) United States
(12) Patent Application Publication
(10) Pub. No.: US 2008/0108712 A1
(43) Pub. Date: May 8, 2008

(54) NUCLEOTIDE SEQUENCE ASSOCIATED WITH ACUTE CORONARY SYNDROME AND MORTALITY

(75) Inventors: Sharon Cresci, St. Louis, MO (US); Howard L. McLeod, Chapel Hill, NC (US); Daniel P. Kelly, St. Louis, MO (US); John A. Spertus, Kansas City, MO (US)

Correspondence Address: POLSINELLI SHALTON FLANIGAN SUELTHAUS PC, 100 SOUTH FOURTH STREET, SUITE 100, SAINT LOUIS, MO, 63102-1825, US

(73) Assignee: WASHINGTON UNIVERSITY IN ST. LOUIS, St. Louis, MO (US)

(21) Appl. No.: 11/862,794

(22) Filed: Sep. 27, 2007

Related U.S. Application Data
(60) Provisional application No. 60/827,126, filed on Sep. 27, 2006.
(60) Provisional application No. 60/864,447, filed on Nov. 6, 2006.

(51) Int. Cl. A61K 47/00; C12Q 1/68; C07H 21/04; C40B 40/08; A61P 7/00

(52) U.S. Cl. 514/789; 435/6; 536/23.1; 506/17

(57) ABSTRACT

The present invention provides methods for diagnosing and/or treating subjects that either have or are at risk for developing acute coronary syndrome. In addition, the invention provides isolated nucleotide sequences and arrays comprising the nucleotide sequences that may be used for treatment and/or diagnosis of subjects that either have or are at risk for developing acute coronary syndrome.

### CROSS-REFERENCE TO RELATED APPLICATIONS

This application claims priority to U.S. Provisional Application Nos. 60/827,126, which was filed on Sep. 27, 2006, and 60/864,447, which was filed on Nov. 6, 2006, each of which is hereby incorporated by reference in their entirety.

### FIELD OF THE INVENTION

The present invention generally relates to methods for diagnosing and/or treating subjects that either have or are at risk for developing acute coronary syndrome. In addition, the invention provides isolated nucleotide sequences and arrays comprising the nucleotide sequences that may be used for treatment and/or diagnosis of subjects that either have or are at risk for developing acute coronary syndrome.

### BACKGROUND OF THE INVENTION

Diabetes mellitus (DM) affects approximately 16 million people in the United States. Among diabetic patients, the macrovascular complications of accelerated atherosclerosis, particularly acute coronary events, remain the principal cause of death. Among patients hospitalized with acute coronary syndrome (ACS) in the U.S. each year, those (˜30%) with DM have significantly increased rates of morbidity and mortality. In addition, patients with DM have a poorer response to current therapies and a high frequency of recurrent events. The mechanism by which DM confers worse outcome for patients with ACS remains unknown. It is not only possible, but also likely, that metabolic and genetic differences in patients with DM, compared to those without, may account for these differences.

Peroxisome proliferator-activated receptor alpha protein (PPARα, which is encoded by the PPARA gene) is highly expressed in the heart and other tissues, such as liver and skeletal muscle, that rely on fatty acid oxidation as their primary energy substrate. PPARα is thought of as the central regulator of genes involved in fatty acid metabolism and appears to mediate the balance between cellular fatty acid metabolism and glucose homeostasis, particularly at times of metabolic or physiologic stress. Emerging evidence suggests that PPARα contributes to the metabolic and phenotypic derangements seen in DM and diabetic cardiomyopathy, and PPARA polymorphisms have been associated with the incidence of type 2 DM, obesity, insulin resistance, and abnormal lipid profiles.

Because of the high incidence of DM, it would be desirable to identify those patients at increased risk for coronary events and modify their course of treatment accordingly. Since PPARα plays a key role in cardiac metabolism and considering that PPARA is polymorphic, single nucleotide polymorphisms (SNPs) within regions of the PPARA gene may provide such a diagnostic indicator.

### SUMMARY OF THE INVENTION

One aspect of the invention provides a method for identifying a subject at risk for increased mortality associated with acute coronary syndrome. The method comprises determining the identity of the nucleotide at position −54,642 of a nucleotide sequence comprising the nucleic acid sequence of SEQ ID NO:1 in a sample from a subject. The presence of a G instead of an A at nucleotide −54,642 indicates a risk for increased mortality.

In another aspect, the invention encompasses a method for treating acute coronary syndrome in a subject. The method comprises determining the identity of the nucleotide at position −54,642 of a nucleotide sequence comprising the nucleotide sequence of SEQ ID NO:1 in a sample from a subject. If nucleotide −54,642 is a G, the subject is administered treatment to modulate the activity of PPARα.

In yet another aspect, the invention provides an isolated nucleic acid comprising the nucleotide sequence of SEQ ID NO:3.

A further aspect of the invention provides an isolated nucleic acid comprising at least ten contiguous nucleotides, including nucleotide −54,642, of a nucleotide sequence selected from the group consisting of SEQ ID NO:3, SEQ ID NO:5, and SEQ ID NO:7.

In an additional aspect, the invention provides an array. The array comprises a substrate having at least one address, wherein at least one address has disposed thereon a nucleic acid that can specifically bind to SEQ ID NO:3 or a portion thereof containing nucleotide −54,642.

Other aspects and iterations of the invention are described more fully herein.

### FIGURE LEGENDS

FIG. 1 presents Kaplan-Meier estimates of mortality stratified by PPARA −54,642 genotype in patients with (upper) and without (lower) DM. p-values between genotype groups are shown. (p-value for genotype by diabetes interaction=0.008).

FIG. 2 depicts a graph of 3-year mortality in patients with DM stratified by PPARA −54,642 and −35,014 genotype (4 “bad” alleles v. 0 “bad alleles). (HR=5.7, CI 1.2-26.4; p=0.03). “Other” represents allele combinations other than CC/AA, and AA/GG. “N at risk” refers to the number of subjects represented in each genotype category.

FIG. 3 depicts images and a graph illustrating that ERR binds to the −54,642 SNP. (A) Electrophorectic mobility shift assay of binding activity performed with ³²P-labeled probes differing at the PPARA −54,642 SNP sites (with the corresponding linked −54,645 SNP) using recombinant ERRα and ERRγ proteins. (B) Competition experiments, using 10-fold, 50-fold and 100-fold excess of unlabeled G probe or unlabeled A probe as a competitor, demonstrate that more of the cold A probe, compared to the cold G probe, is necessary to compete with labeled G probe for binding to ERRα (demonstrating lower relative binding affinity of A vs. G). Mean relative band intensities (representative trial, top) from 3 trials were quantified by phosphorimage analysis and results are depicted graphically in the bottom panel. Asterisks represent significantly different binding to probe compared to control (p<0.05).

FIG. 4 illustrates transcriptional activation of −54,642 SNP. Mean normalized luciferase activities (±SE) in (left) C2C12 myoblast cells or (right) CV1 cells cotransfected with PPARA −54,642 A or PPARA −54,642 G promoter-reporter constructs. A schematic of the promoter-reporter constructs comprising two copies of either the G or the A variant element (ERRE) is shown at the top of the figure. Solid bars represent expression in the presence of a mammalian expression vector that overexpresses ERRα. Stippled bars represent expression in the presence of a vector control. Asterisks represent significantly different transcriptional activation compared to vector control (p<0.05). Four independent trials were performed in triplicate for each cell line.

### DETAILED DESCRIPTION OF THE INVENTION

In accordance with the present invention, it has been discovered that three single nucleotide polymorphisms (SNP) in the promoter region of the PPARA gene are correlated with an increased risk of mortality associated with acute coronary syndrome. The SNPs are located at −54,642, −54,645, and −35,014 of the PPARA gene, and may be correlated both individually and in combination with an increased risk of mortality associated with acute coronary syndrome.

Generally speaking, the present invention encompasses sequences comprised of one or more of the three SNPs and methods of using the genotype of one or more of the three SNPs of a subject to predict the risk of mortality from acute coronary syndrome for a subject, to administer treatment, and to decrease mortality.

I. Isolated Nucleic Acids of the Invention

One aspect of the present invention is an isolated nucleic acid comprising the nucleotide sequence of SEQ ID NO:3. SEQ ID NO:3 represents the nucleotide sequence of the PPARA gene (SEQ ID NO:1) with a single nucleotide polymorphism in promoter A, such that nucleotide −54,642 is a G instead of an A. The nucleotide position numbers used herein are in reference to the translational start site of the PPARA gene. Negative nucleotide position numbers are upstream of the translational start site, while positive nucleotide position numbers are downstream of the translational start site. In one embodiment, the isolated nucleic acid comprises at least 20, 30, 40, 50, 60, 70, 80, 90, or 100 contiguous nucleotides of the nucleotide sequence of SEQ ID NO:3, including nucleotide −54,642. In yet another embodiment, the isolated nucleic acid comprises at least 120, 140, 160, 180, 200, 220, 240, 260, 280 or 300 contiguous nucleotides of the nucleotide sequence of SEQ ID NO:3, including nucleotide −54,642. In still another embodiment, the isolated nucleic acid comprises at least 325, 350, 375, 400, 425, 450, 475, 500, 525 or 550 contiguous nucleotides of the nucleotide sequence of SEQ ID NO:3, including nucleotide −54,642. In a further embodiment, the isolated nucleic acid comprises at least a 1000, 5000, 10,000, 15,000, or 20,000 contiguous nucleotides of the nucleotide sequence of SEQ ID NO:3, including nucleotide −54,642. In an alternative, the isolated nucleic acid comprises the nucleotide sequence of SEQ ID NO:2, except for nucleotide −54,642 where a G replaces the A.

The isolated nucleic acid of each of the above embodiments may also include nucleotide −54,645. In one embodiment, the isolated nucleic acid is comprised of the nucleotide sequence of SEQ ID NO:1, except that the nucleotide at position −54,645 is a C instead of a T. In other words, one embodiment is the isolated nucleic acid comprising the nucleotide sequence of SEQ ID NO:4. In an exemplary embodiment, the isolated nucleic acid is comprised of the nucleotide sequence of SEQ ID NO:1, except for a G at nucleotide −54, 642 and a C at nucleotide −54,645. Stated another way, an exemplary embodiment is the isolated nucleic acid comprising the nucleotide sequence of SEQ ID NO:5. In one embodiment, the isolated nucleic acid comprises at least 20, 30, 40, 50, 60, 70, 80, 90, or 100 contiguous nucleotides of the nucleotide sequence of SEQ ID NO:4 or SEQ ID NO:5, including nucleotide −54,642. In yet another embodiment, the isolated nucleic acid comprises at least 120, 140, 160, 180, 200, 220, 240, 260, 280 or 300 contiguous nucleotides of the nucleotide sequence of SEQ ID NO:4 or SEQ ID NO:5, including nucleotide −54,642. In still another embodiment, the isolated nucleic acid comprises at least 325, 350, 375, 400, 425, 450, 475, 500, 525 or 550 contiguous nucleotides of the nucleotide sequence of SEQ ID NO:4 or SEQ ID NO:5, including nucleotide −54,642. In a further embodiment, the isolated nucleic acid comprises at least a 1000, 5000, 10,000, 15,000, or 20,000 contiguous nucleotides of the nucleotide sequence of SEQ ID NO:4 or SEQ ID NO:5.

The isolated nucleic acid of each of the above embodiments may additionally include nucleotide −35,014. In one embodiment, the isolated nucleic acid is comprised of the nucleotide sequence of SEQ ID NO:1, except that nucleotide −35,014 is an A instead of a C. In other words, one embodiment is a nucleic acid comprising the nucleotide sequence of SEQ ID NO:6. In another embodiment, the isolated nucleic acid is comprised of the nucleotide sequence of SEQ ID NO:3 except that nucleotide −35,014 is an A instead of a C. In yet another embodiment, the isolated nucleic acid is comprised of the nucleotide sequence of SEQ ID NO:4 except that nucleotide −35,014 is an A instead of a C. In a further embodiment, the isolated nucleic acid may be comprised of the nucleotide sequence of SEQ ID NO:1, except for a G at nucleotide −54,642, a C at nucleotide −54,645, and an A at nucleotide −35,014 (represented by SEQ ID NO:7). In one embodiment, the isolated nucleic acid comprises at least 20, 30, 40, 50, 60, 70, 80, 90, or 100 contiguous nucleotides of the nucleotide sequence of SEQ ID NO:6 or SEQ ID NO:7 including nucleotide −35,014 and/or nucleotide −54,642. In yet another embodiment, the isolated nucleic acid comprises at least 120, 140, 160, 180, 200, 220, 240, 260, 280 or 300 contiguous nucleotides of the nucleotide sequence of SEQ ID NO:6 or SEQ ID NO:7, including nucleotide −35,014 and/or nucleotide −54,642. In still another embodiment, the isolated nucleic acid comprises at least 325, 350, 375, 400, 425, 450, 475, 500, 525 or 550 contiguous nucleotides of the nucleotide sequence of SEQ ID NO:6 or SEQ ID NO:7 including nucleotide −35,014 and/or nucleotide −54,642. In a further embodiment, the isolated nucleic acid comprises at least a 1000, 5000, 10,000, 15,000, or 20,000 contiguous nucleotides of the nucleotide sequence of SEQ ID NO:6 or SEQ ID NO:7 including nucleotide −35,014 and/or nucleotide −54,642.

The present invention also encompasses nucleic acids that are complementary to the isolated nucleic acid sequences described above. In one embodiment, the nucleic acid hybridizes to a nucleic acid comprising the nucleotide sequence of SEQ ID NO:3 but not to a nucleic acid consisting of the nucleotide sequence of SEQ ID NO:1. In another embodiment, the nucleic acid hybridizes to a nucleic acid molecule comprising the nucleotide sequence of SEQ ID NO:4 but not to a nucleic acid consisting of the nucleotide sequence of SEQ ID NO:1. In yet another embodiment, the nucleic acid hybridizes to a nucleic acid molecule comprising the nucleotide sequence of SEQ ID NO:5 but not to a nucleic acid consisting of the nucleotide sequence of SEQ ID NO:1. In still yet another embodiment, the nucleic acid hybridizes to a nucleic acid comprising the nucleotide sequence of SEQ ID NO:6 but not to a nucleic acid consisting of the nucleotide sequence of SEQ ID NO:1. In an alternative embodiment, the nucleic acid hybridizes to a nucleic acid comprising the nucleotide sequence of SEQ ID NO:7 but not to a nucleic acid molecule consisting of the nucleotide sequence of SEQ ID NO:1.

The above nucleic acids may also be combined with each other, or with other nucleic acids. For instance, a nucleic acid that hybridizes with a nucleic acid comprising the nucleotide sequence of SEQ ID NO:3, 4, or 5 may be combined with a nucleic acid that hybridizes with a nucleic acid comprising the nucleotide sequence of SEQ ID NO:6. Alternatively, a nucleic acid that hybridizes with a nucleic acid comprising the nucleotide sequence of SEQ ID NO:3, 4, 5, 6, or 7 may be combined with a nucleic acid that hybridizes to a nucleic acid comprising the nucleotide sequence of SEQ ID NO:1 or 2. In another alternative, a nucleic acid that hybridizes to a nucleic acid comprising the nucleotide sequence of SEQ ID NO: 3, 4, 5, 6, or 7 may be combined with a nucleic acid that hybridizes with a nucleic acid sequence of a gene besides PPARA.

Hybridization of nucleic acids is typically performed under stringent conditions. Nucleic acid duplex or hybrid stability is expressed as the melting temperature or Tm, which is the temperature at which a probe dissociates from a target DNA. This melting temperature is used to define the required stringency conditions. To maximize the rate of annealing of the probe with its target, hybridizations are generally carried out at a temperature that is about 20 to 25° C. below the Tm. For instance, stringent conditions may typically involve hybridizing at about 68° C. in 5× SSC/5×Denhardt's solution/1.0% SDS, and washing in 0.2×SSC/0.1% SDS at about 68° C. Moderately stringent conditions include washing in 3×SSC at 42° C. The parameters of salt concentration and temperature can be varied to achieve the optimal level of identity between the nucleic acid and the target sequence, for instance, a sequence comprising the nucleotide sequence of SEQ ID NO:3, SEQ ID NO:4, SEQ ID NO:5, SEQ ID NO:6, or SEQ ID NO:7. One skilled in the art will appreciate which parameters to manipulate to optimize hybridization. Additional guidance regarding such conditions is readily available in the art, for example, by Sambrook et al., 1989, Molecular Cloning, A Laboratory Manual, Cold Spring Harbor Press, N.Y.; and Ausubel et al., (eds.), 1995, Current Protocols in Molecular Biology, (John Wiley & Sons, N.Y.) at Unit 2.10.

The isolated nucleic acids of the invention may be labeled. Non-limiting examples of suitable labels include fluorescent labels, chemiluminescent labels, radioactive labels, colorimetric labels, and resonance labels. Methods of labeling probes are well known in the art.

The various nucleic acids mentioned above may be obtained using a variety of different techniques known in the art. The nucleic acids may be isolated using standard techniques, or may be purchased or obtained from a depository. Once the nucleic acid is obtained, it may be amplified and/or sequenced for use in a variety of applications, e.g. the methods described below.

The invention also encompasses production of nucleic acids comprising the nucleotide sequence of SEQ ID NO:3, SEQ ID NO:4, SEQ ID NO:5, SEQ ID NO:6, or SEQ ID NO:7, derivatives, or fragments thereof, that may be made by any method known in the art, including by synthetic chemistry. After production, the synthetic sequence may be inserted into any of the many available expression vectors and cell systems using reagents well known in the art.

II. Methods of Using the Isolated Nucleic Acids

(a) Determining the Identity of the SNP Nucleotides in a Subject

One aspect of the present invention encompasses using the isolated nucleic acids of the invention to determine the identity of a nucleotide at position −54,642, −54,645, or −35,014, or any combination thereof, in a subject. In certain embodiments, the invention encompasses determining the identity of a nucleotide at position −54,642, −54,645, or −35,014, or any combination thereof, of an isolated nucleic acid of the invention derived from one or both chromosomes of a subject.

Determining the identity of nucleotide −54,642, −54,645, or −35,014, or any combination thereof, may be performed using techniques commonly known in the art. Generally speaking, the method comprises collecting a biological sample from a subject, isolating DNA from the biological sample, and sequencing the sample. Typically, the subject is human. In certain embodiments, the subject may be a diabetic human. In other embodiments, the subject may be at risk for acute coronary syndrome. As used herein, “at risk” refers to a subject that has at least one risk factor for acute coronary syndrome. Risk factors may include diabetes (including type I and type II), angina (including stable, non-stable, and variant angina) smoking history, hypertension, hypotension, male gender, increased age, hypercholesterolemia, hyperlipidemia, prior cerebrovascular accident (CVA), inherited metabolic disorders, methamphetamine use, occupational stress, and connective tissue disease. In another embodiment, the subject may be on hormone replacement therapy.

As disclosed above, the method of determining the identity of nucleotide −54,642, −54,645, or −35,014, or any combination thereof in a subject includes, in part, collecting a biological sample from a subject. Non-limiting examples of biological samples may include fluid samples, biopsy samples, skin samples, and hair samples. Fluid samples may include blood, saliva, tears, and lymph. For instance, DNA may be isolated from a blood sample, a saliva sample, an epithelial sample, a skin sample, a hair sample, or other biological sample commonly used in the art. Methods of collecting a biological sample from a subject are well known in the art. In particular, methods of collecting blood samples, saliva samples, epithelial samples, and skin samples are well known in the art.

DNA may be isolated from a biological sample using methods commonly known in the art. A skilled artisan would appreciate that the method of DNA isolation can and will vary depending on the biological sample used. For more information, see Maniatis at chapter 2. Additionally, commercially available kits such as Mini, Midi, or Maxi Preps, from Qiagen or commercially available reagents such as Trizol reagent from Invitrogen may be used to isolate DNA from a biological sample.

In an exemplary embodiment, the isolated DNA encompasses an isolated nucleic acid of the invention, such that the nucleic acid is comprised of nucleotide −54,642 of the nucleic acid sequence of SEQ ID NO: 1, SEQ ID NO:3, SEQ ID NO:4, SEQ ID NO:5, SEQ ID NO:6, or SEQ ID NO:7. In another embodiment, the isolated DNA encompasses an isolated nucleic acid of the invention, such that the nucleic acid is comprised of nucleotide −54,645 of the nucleic acid sequence of SEQ ID NO: 1, SEQ ID NO:3, SEQ ID NO:4, SEQ ID NO:5, SEQ ID NO:6, or SEQ ID NO:7. In yet another embodiment, the isolated DNA encompasses an isolated nucleic acid of the invention, such that the nucleic acid is comprised of nucleotide −35,014 of the nucleic acid sequence of SEQ ID NO: 1, SEQ ID NO:3, SEQ ID NO:4, SEQ ID NO:5, SEQ ID NO:6, or SEQ ID NO:7. In still yet another embodiment, the isolated DNA encompasses an isolated nucleic acid of the invention, such that the nucleic acid is comprised of nucleotide −54,462 and −54,645 of the nucleic acid sequence of SEQ ID NO: 1, SEQ ID NO:3, SEQ ID NO:4, SEQ ID NO:5, SEQ ID NO:6, or SEQ ID NO:7. In another exemplary embodiment, the isolated DNA encompasses an isolated nucleic acid of the invention, such that the nucleic acid is comprised of nucleotide −54,462 or −54,645, and nucleotide −35,014 of the nucleic acid sequence of SEQ ID NO: 1, SEQ ID NO:3, SEQ ID NO:4, SEQ ID NO:5, SEQ ID NO:6, or SEQ ID NO:7.

The isolated DNA may then be sequenced using techniques commonly known in the art. For instance, see Maniatis, chapter 7, 2003. In one embodiment chain termination sequencing is employed to determine the identity of the nucleotide at position at −54,642, −54,645, or −35,014. In another embodiment, the nucleic acid sample is sequenced using pyrosequencing.

(b) Identifying a Subject at Risk for Increased Mortality Associated with Acute Coronary Syndrome

Another aspect of the present invention encompasses a method of identifying a subject at risk for increased mortality associated with acute coronary syndrome. The method comprises, in part, determining the identity of the nucleotide at position −54,642 of a nucleic acid of the invention in a biological sample from a subject. The presence of a G, instead of an A, at position −54,642 may indicate an increased risk for mortality associated with acute coronary syndrome. For a given nucleotide position, a subject may be homozygous or heterozygous. As used herein, homozygous refers to a subject that has the same nucleotide at a given position on both chromosomes. Heterozygous, as used herein, refers to a subject that has different nucleotides at a given position on each chromosome. Generally speaking, a subject that is homozygous for G at position −54,642 has a greater risk for mortality associated with acute coronary syndrome than a heterozygous subject. Similarly, a subject that is heterozygous at position −54,642 has a greater risk for mortality associated with acute coronary syndrome than subject that is homozygous for A at position −54,642.

For instance, as detailed in the examples, a diabetic subject homozygous for G at position −54,642 has an approximately five fold greater risk of mortality associated with acute coronary syndrome after three years than a diabetic subject homozygous for A at position −54,642. Similarly, a diabetic subject heterozygous at position −54,642 has an approximately 1.3 fold greater risk of mortality associated with acute coronary syndrome after three years than a diabetic subject homozygous for A at position −54,642. Alternatively, a subject homozygous for G at position −54,642 who is on hormonal replacement therapy may have an increased risk of mortality associated with acute coronary syndrome than a subject homozygous for A at position −54,642 on hormonal replacement therapy.

The method may further comprise identifying the nucleotide at position −54,645 of a nucleic acid of the invention in a biological sample from a subject. The presence of a C, instead of a T, at position −54,645 may indicate an increased risk for mortality associated with acute coronary syndrome. Generally speaking, a subject that is homozygous for C at position −54,645 has a greater risk for mortality associated with acute coronary syndrome than a heterozygous subject. Similarly, a subject that is heterozygous at position −54,645 has a greater risk for mortality associated with acute coronary syndrome than a subject that is homozygous for T at position −54,645.

Additionally, whether a subject is homozygous or heterozygous with regard to position −54,642 and −54,645 may be considered together to identify a subject at risk for increased mortality associated with acute coronary syndrome. For instance, a subject homozygous for G at position −54,642 and homozygous for C at position −54,645 may have an increased risk of mortality associated with acute coronary syndrome compared to a subject heterozygous at both positions −54,642 and −54,645. Similarly, a subject heterozygous at position −54,642 and position −54,645 may have an increased risk of mortality associated with acute coronary syndrome than a subject homozygous for A at position −54,642 and homozygous for T at position −54,645.

The method may further comprise identifying the nucleotide at position −35,014 of a nucleic acid of the invention in a biological sample from a subject. The presence of an A, instead of a C, at position −35,014 may indicate an increased risk for mortality associated with acute coronary syndrome. Generally speaking, a subject that is homozygous for A at position −35,014 has a greater risk for mortality associated with acute coronary syndrome than a heterozygous subject. Similarly, a subject that is heterozygous at position −35,014 has a greater risk for mortality associated with acute coronary syndrome than subject that is homozygous for C at position −35,014.

Whether a subject is homozygous or heterozygous with regard to position −54,642, −54,645, −35,014, or any combination thereof, may be considered together to identify a subject at risk for increased mortality associated with acute coronary syndrome. For instance, a subject homozygous for G at position −54,642, homozygous for C at position −54,645, and homozygous for A at position −35,014 may have an increased risk of mortality associated with acute coronary syndrome than a subject heterozygous at position −54,642, −54,645, and −35,014. Similarly, a subject heterozygous at position −54,642, heterozygous at position −54,645, and heterozygous at position −35,014 may have an increased risk of mortality associated with acute coronary syndrome than a subject homozygous for A at position −54,642, homozygous for T at position −54,645, and homozygous for C at position −35,014. Other combinations of homozygosity and heterozygosity at positions −54,642, −54,645, and −35,014 may also be used to determine risk of mortality associated with acute coronary syndrome in a subject.

(c) Method of Treating Acute Coronary Syndrome

A further aspect of the invention encompasses a method of treating acute coronary syndrome in a subject. The method comprises, in part, determining the identity of the nucleotide position −54,642, −54,645, −35,014, or any combination thereof, in a nucleic acid of the invention from biological sample of a subject. If the subject is homozygous for G at position −54,642, homozygous for C at position −54,645, homozygous for A at position −35,014, or heterozygous at position −54,642, −54,645, or −35,014, then the subject may be administered treatment to modulate the activity of PPARα.

Whether a subject is homozygous or heterozygous at position −54,642, −54,645, or −35,014 may be used in combination to determine if a subject may be administered treatment to modulate the activity of PPARα. For instance, a subject homozygous for G at position −54,642, homozygous for C at position −54,645, and homozygous for A at position −35,014 may be administered treatment to modulate the activity of PPARα. Similarly, a subject heterozygous at position −54,642, −54,645, and −35,014 may be administered treatment to modulate the activity of PPARα.

i. Treatments to Modulate the Activity of PPARα

Treatments to modulate the activity of PPARα include treatments that increase PPARα activity, treatments that decrease PPARα activity, and treatments that maintain PPARα activity. As used herein, “activity of PPARα” includes rates of transcription of the PPARA gene, rates of translation of PPARα, and regulatory function of PPARα. In certain embodiments, modulating the rate of transcription of the PPARA gene modulates the activity of PPARα. In one embodiment, the transcription rate of the PPARA gene is modulated by altering the binding of an estrogen related receptor to promoter A of PPARA, as discussed in more detail below. In some embodiments, modulating the rate of translation of PPARα modulates the activity of PPARα.

Methods of altering the binding of an estrogen related receptor include modulating the concentration of an estrogen related receptor or inhibiting the binding of an estrogen related receptor to promoter A of the PPARA gene. Estrogen related receptors (ERR) include ERRα, ERRβ, and ERRγ. In an exemplary embodiment, the ERR may be ERRα or ERRγ. In one embodiment, the transcription of the PPARA gene is modulated by increasing the concentration of an estrogen related receptor in the subject. In another embodiment, the transcription of the PPARA gene is modulated by decreasing the concentration of an estrogen related receptor in the subject. Possible methods of modulating the concentration of estrogen related receptor is known in the art.

Altering the binding of an estrogen related receptor might also include inhibiting the binding of an estrogen related receptor to promoter A of the PPARA gene. Methods of inhibiting the binding of an estrogen related receptor to promoter A are well known in the art. For instance, microRNA may be used to block the binding site of the estrogen related receptor.

In other embodiments, the activity of PPARα is modulated by modulating the regulatory function of PPARα. Methods of modulating the regulatory function of PPARα are known in the art. For instance, PPARα agonists may be used to modulate the regulatory function of PPARα. PPARα agonists may be specific PPARα agonists, in that they only modulate PPARα regulatory function and not PPARγ or PPARδ regulatory function. Alternatively, PPARα agonists may be non-specific, in that they modulate PPARα regulatory function and may also modulate PPARγ or PPARδ regulatory function. Non-limiting examples of PPARα agonists (both specific and non-specific) include fibrates, such as gemfibrozil, thizaolidinediones, such as pioglitzaone, and glitazars, such as ragaglitazar.

PPARα antagonists may also be used to modulate PPARα regulatory function. Alternatively, instead of modulating PPARα regulatory function, PPARγ or PPARδ regulatory function may be modulated. Administering a PPARγ or PPARδ agonist may be appropriate when it is desirable to maintain PPARα regulatory function. For instance, it may be desirable to maintain PPARα regulatory function during acute coronary syndrome in a subject. Non-limiting examples of PPARγ or PPARδ agonists include rosiglitazone.

ii. Methods of Administering Treatment

As will be appreciated by the skilled artisan, the therapeutic agents of the present invention may be formulated into pharmaceutical compositions and administered by a number of different means that will deliver a therapeutically effective dose. They may be administered locally or systemically. Such compositions may be administered orally, parenterally, by inhalation spray, intrapulmonary, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, intraarterial, intraperitoneal, intracochlear, or intrasternal injection, or infusion techniques. The therapeutic agents of the present invention may be administered by daily subcutaneous injection or by implants. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Injectable preparations, for example, sterile injectable aqueous or oleaginous suspensions, may be formulated according to the known art using suitable dispersing or wetting agents and suspending agents. The sterile injectable preparation may also be a sterile injectable solution or suspension in a nontoxic parenterally acceptable diluent or solvent. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols may be used. Mixtures of solvents and wetting agents such as those discussed above are also useful.

Solid dosage forms for oral administration may include capsules, tablets, pills, powders, and granules. In such solid dosage forms, the compounds are ordinarily combined with one or more adjuvants appropriate to the indicated route of administration. If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as can be provided in a dispersion of active compound in hydroxypropylmethyl cellulose. In the case of capsules, tablets, and pills, the dosage forms may also comprise buffering agents such as sodium citrate, or magnesium or calcium carbonate or bicarbonate. Tablets and pills may additionally be prepared with enteric coatings.

For therapeutic purposes, formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

The amount of active ingredient that may be combined with the carrier materials to produce a single dosage of a therapeutic agent of the invention will vary depending upon the patient and the particular mode of administration. In general, the pharmaceutical compositions may contain a therapeutic agent in the range of about 1 to 1000 mg, more typically, in the range of about 10 to 800 mg and still more typically, between about 50 and 600 mg. A daily dose of about 1 to 4000 mg, or more typically, between about 10 and 3000 mg, and even more typically, from about 100 to 2500 mg, may be appropriate. The daily dose depends on various factors, particularly the age and body weight of the subject. A reduced dosage is generally administered to children. The daily dose is generally administered in one to about four doses per day.

Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

Treatment to modulate PPARα activity may be administered before, during or after acute coronary syndrome in a subject. In one embodiment, treatment to modulate PPARα activity is administered during acute coronary syndrome in a subject. In another embodiment, treatment to modulate PPARα activity is administered after acute coronary syndrome in a subject. In yet another embodiment, treatment to decrease PPARα activity is administered during acute coronary syndrome in a subject. In still yet another embodiment, treatment to increase PPARα activity is administered after acute coronary syndrome in a subject.

iii. Decreasing Mortality Associated with Acute Coronary Syndrome

Treating acute coronary syndrome by modulating PPARα activity in subjects may also aid in decreasing mortality associated with acute coronary syndrome. In other words, administering treatment to modulate the activity of PPARα in subjects who are homozygous for G at position −54,642, homozygous for C at position −54,645, homozygous for A at position −35,014, or heterozygous at position −54,642, −54,645, or −35,014, or any combination thereof, may decrease the increased risk of mortality associated with acute coronary syndrome in these subjects.

iv. Diagnosing Acute Coronary Syndrome in a Subject

Methods of diagnosing acute coronary syndrome in a subject are well known in the art. For more information, see Achar et al., “Diagnosis of Acute Coronary Syndrome,” Am Fam Physician 2005; 72:119026, hereby incorporated by reference.

III. Arrays

A further aspect of the invention is a nucleic acid array. The nucleic acid array is comprised of a substrate having at least one address. Nucleic acid arrays are commonly known in the art, and moreover, substrates that comprise nucleic acid arrays are also well known in the art. Non-limiting examples of substrate materials include glass and plastic. A substrate may be shaped like a slide or a chip (i.e. a quadrilateral shape), or alternatively, a substrate may be shaped like a well.

The array of the present invention is comprised of at least one address, wherein the address has disposed thereon a nucleic acid that can specifically bind to the nucleic acid sequence of SEQ ID NO:3 or a portion thereof containing nucleotide −54,642. In another embodiment, the array is comprised of at least one address, wherein the address has disposed thereon a nucleic acid that can specifically bind to the nucleic acid sequence of SEQ ID NO:4 or a portion thereof containing nucleotide −54,645. In yet another embodiment, the array is comprised of at least one address, wherein the address has disposed thereon a nucleic acid that can specifically bind to the nucleic acid sequence of SEQ ID NO:6 or a portion thereof containing nucleotide −35,014. In still yet another embodiment, the array is comprised of at least one address, wherein the address has disposed thereon a nucleic acid that can specifically bind to the nucleic acid sequence of SEQ ID NO:5 or a portion thereof including nucleotide −54,642 or −54,645.

In a further embodiment, the array is comprised of at least two addresses, wherein at least one address has disposed thereon a nucleic acid that can specifically bind to the nucleic acid sequence of SEQ ID NO:3 or a portion thereof containing nucleotide −54,642 and at least one address has disposed thereon a nucleic acid that can specifically bind to the nucleic acid sequence of SEQ ID NO:6 or a portion thereof containing nucleotide −35,014. In yet a further embodiment, the array is comprised of at least two addresses, wherein at least one address has disposed thereon a nucleic acid that can specifically bind to the nucleic acid sequence of SEQ ID NO:5 or a portion thereof including nucleotide −54,642 or −54,645 and at least one address has disposed thereon a nucleic acid that can specifically bind to the nucleic acid sequence of SEQ ID NO:6 or a portion thereof containing nucleotide −35,014.

An array typically is comprised from between about 1 to about 10,000 addresses. In one embodiment, the array is comprised from between about 10 to about 8,000 addresses. In another embodiment, the array is comprised of no more than 500 addresses. In an alternative embodiment, the array is comprised of no less than 500 addresses.

### DEFINITIONS

As used herein, “acute coronary syndrome” refers to myocardial infarction and unstable angina, but may also include new-onset angina and sudden cardiac death. Acute coronary syndrome may result from atherosclerosis or coronary artery disease, wherein the instability and subsequent rupture of atherosclerotic plaques may lead to thrombotic occlusion of coronary arteries. Acute coronary syndrome may also develop as a result of a heart condition.

As used herein, “heart condition” refers to a wide range of abnormalities and/or diseases of the heart, coronary vasculature, or blood vessels surrounding the hear including underlying conditions, such as, ischemia (including, for example, atherosclerosis (coronary artery disease), embolism, congenital heart defects, anemia, lung disease, and abnormal stimulation (e.g. sympathomimetic abuse)), hypertension (including, for example, systemic hypertension (e.g. primary and secondary) and pulmonary hypertension (e.g. chronic obstructive pulmonary disease, restrictive lung disease, pulmonary embolism, and morbid obesity)), valvular disease (including, for example, mitral valve disease, aortic valve disease, tricuspid valve disease and pulmonary valve disease), heart muscle disease (including, for example, ischemic cardiomyopathy, dilated cardiomyopathy, hypertensive cardiomyopthay, hypertrophic cardiomyopathy, restrictive cardiomyopathy, and specific heart muscle disease resulting from cardiac infection (i.e. bacterial or viral infection), toxins, metabolites, neuromuscular disease, storage disorders, infiltration disorders, and immunologic disorder), pericardial disease, rheumatoid heart disease, neoplastic heart disease (including, for example, primary cardiac tumors), coronary vasospasm (including, for example, drug induced vasospasm), cardiac trauma, and genetic or hereditary predisposition that may manifest as angina (including, for example, stable angina, unstable angina, mixed angina, and Prinzmetal's variant angina), myocardial infarction, chronic ischemic heart disease, and sudden cardiac death.

All publications, patents, patent applications and other references cited in this application are herein incorporated by reference in their entirety as if each individual publication, patent, patent application or other reference were specifically and individually indicated to be incorporated by reference.

### EXAMPLES

The following examples illustrate various iterations of the invention.

### Example 1

### PPARA Genotypes are Associated with Increased Mortality in Diabetic ACS Patients in INFORM in a Gene-Dose Dependent Manner

The association between PPARA gene SNPs and adverse cardiovascular outcomes in patients with coronary disease was explored using the INFORM cohort. INFORM is a prospective Kansas City-based registry of ACS patients. This study enrolled 1199 patients (679 patients with a troponin-positive acute MI and 518 with unstable angina and normal troponin; 2 patients were missing information on troponin), 35% with DM, from March 2001 through October 2002. A total of 742 patients were enrolled in the genetic substudy. Two PPARA gene SNPs have been found to have significant associations in INFORM patients with DM, the PPARA −35,014 SNP and the PPARA −54,642 SNP.

DNA was isolated and extracted using the Puregene genomic DNA purification kit (Gentra, Minneapolis, Minn.). The DNA segments containing the region of interest were amplified with the polymerase chain reaction (PCR). PCR primers were designed using Primer3 online software (http://fokker.wi.mit.edu/cgi-bin/primer3/primer3_www.cgi), and pyrosequencing primers were designed using the Pyrosequencing SNP Primer Design Version 1.01 software (http://www.pyrosequencing.com). Before use, PCR primer sequences were screened across the human genome using the NCBI Blast program to ensure their specificity for the gene of interest. PCR was carried out using Amplitaq Gold PCR master mix (ABI, Foster City, Calif.), 1 pmole of each primer (IDT, Coralville, Iowa), and 1 ng DNA. Pyrosequencing was performed using the PSQ HS 96A system with MA v2.0 software. Data were automatically transferred from the PSQ HS 96A to a Microsoft Access database for permanent storage and merging with the clinical datasets through SAS v9.1.

The PPARA −35,014 SNP was originally reported to influence the onset and progression of type 2 diabetes (Flavell et al., 2005 Diabetes 54(2):582-586). When INFORM patients were stratified by DM status, the PPARA −35,014 genotype had an increased association with 3-year mortality in a gene-dose dependent manner (data not shown). Although this association only achieved borderline significance, there was a highly significant genotype by diabetes interaction (p=0.009) for this association.

Genotypes for PPARA −54,642 SNP were successfully obtained in 705 of the 726 (97%) available samples from consecutive ACS patients in the genetic substudy of the INFORM ACS registry. The PPARA −54,642 G allele frequency was 0.70 in Caucasians and 0.40 in African-Americans. A SNP adjacent to the PPARA −54,642 SNP was identified (PPARA −54,645; rs135562) and was found to be in complete LD (D′=1) in all Caucasian and African-American patients in the genetic substudy. PPARA −54,642 G allele frequency in samples from healthy, random blood donors of different ethnicities (97 samples each from Caucasian, African-American, Han Chinese and Mexican populations; G allele frequency of 0.73, 0.32, 0.85, 0.88 respectively) were obtained and were consistent with those found in INFORM for Caucasians and African-Americans. Complete LD (D′=1) between these two SNPs was also observed in these samples. Neither of the variants deviated significantly from Hardy-Weinberg equilibrium.

The ACS population was reevaluated within the context of DM status. When ACS patients with DM were compared by PPARA −54,642 genotype, significant differences were only seen in race, history of prior PCI (19.4% AA vs. 29.3% AG vs. 46.1% GG), admission systolic blood pressure (141.8+/−32.5 AA vs. 143.5+/−25.9 AG vs. 132.4+/−26.5 GG; p=0.03), and receipt of PCI (30.6% AA vs. 44.0% AG vs. 59.6% GG; p=0.02). Of note, there was no significant difference by genotype in age, history of heart failure, or hyperglycemia on admission, factors previously reported to contribute to or be associated with poor prognosis. Given the difference in history of prior PCI and receipt of PCI, the number of diseased vessels with greater than 70% stenosis was assessed in all patients with DM in whom cardiac catheterization data was available (70%) and compared by genotype. No significant differences were found (34.8% vs. 42.6% vs. 36.1% 3 vessel disease; 26.1% vs. 27.8% vs. 37.5% 2 vessel disease; AA vs. AG vs. GG, respectively; p=0.78).

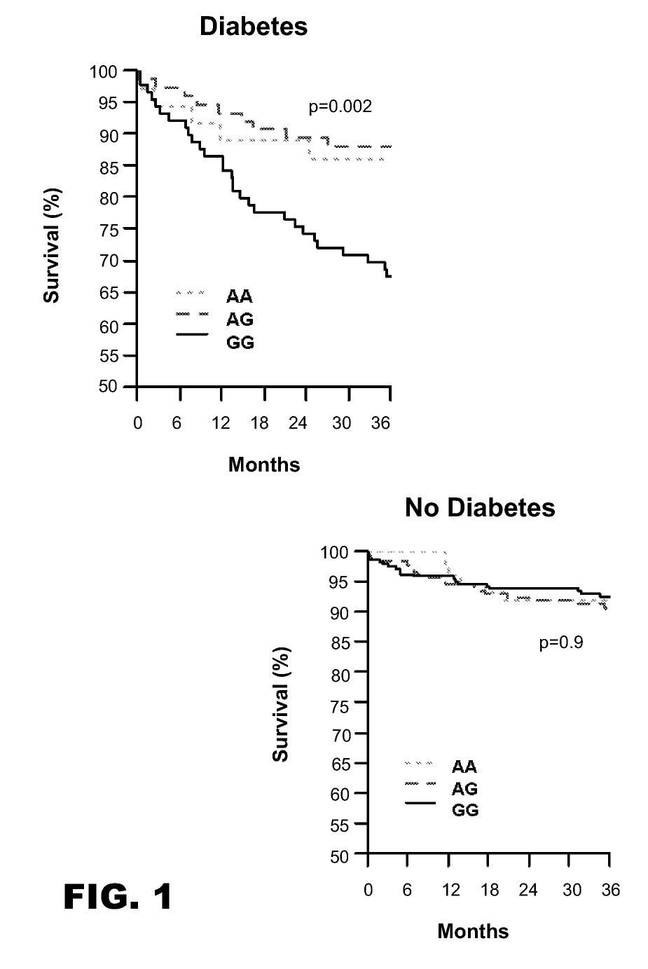

When examined among the entire cohort of ACS patients, PPARA −54,642 genotype was not associated with 3-year mortality. However, PPARA −54,642 genotype was associated with increased 3-year mortality in diabetic (p=0.002) but not in non-diabetic (p=0.90) ACS patients (FIG. 1; genotype by DM interaction p=0.008). Compared with the 8.3% 3-year mortality rate following admission for ACS in patients without diabetes, ACS patients with DM and an A allele at the PPARA −54,642 SNP site had 12.6% 3-year mortality (survival in the AA and AG patients with DM was not significantly different). ACS patients with DM who were homozygous for the GG genotype had 32.7% mortality (FIG. 1; HR 2.85, 95% CI 1.50-5.39; p=0.001).

In multivariable analysis adjusting for age, race, and gender, genotype remained an independent predictor of 3-year mortality. Patients homozygous for the PPARA −54,642 GG genotype had greater than 2-fold relative increase in 3-year mortality (HR 2.49, 95% CI 1.27-4.85; p=0.008), when compared to PPARA −54,642 A allele carriers. Given the racial differences in allele frequency in Caucasians and African-Americans, the effect of PPARA −54,642 genotype on mortality was examined separately in Caucasian DM subjects alone (the largest racial group). The results were consistent with the findings in the entire group. Caucasian patients homozygous for the PPARA −54,642 GG genotype had significantly increased 3-year mortality compared to A allele carriers (HR 2.57, 95% CI 1.19-5.54; p=0.017).

### Example 2

### PPARA −54,642 and −35,014 Genotype is Associated with Increased Mortality in Diabetic ACS Patients in INFORM in a Gene-Dose Dependent Manner

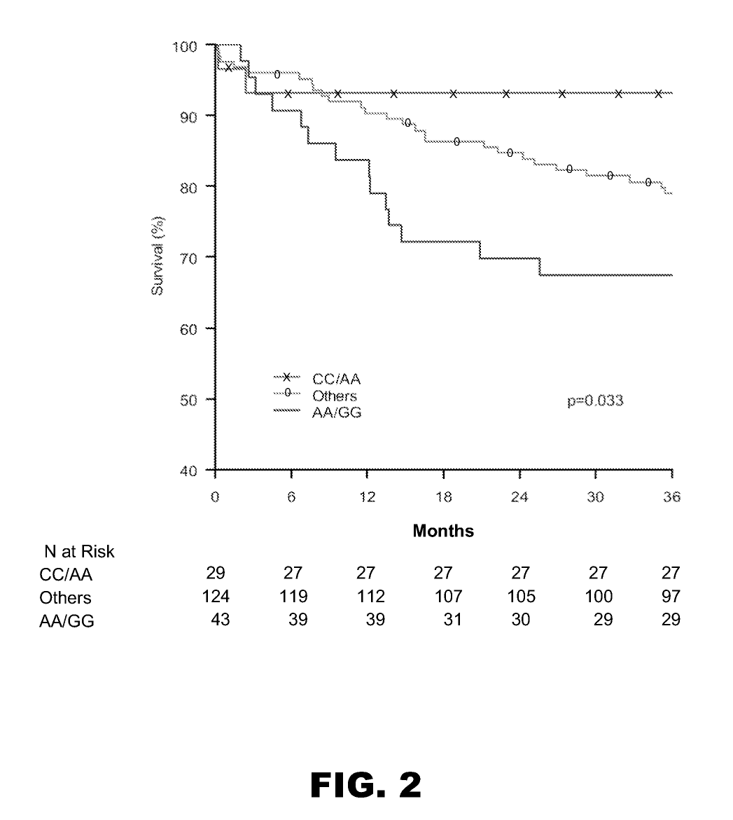

The SNPs at −54,642 and −35,014 work together, such that patients homozygous (GG) at PPARA −54,642 and homozygous (AA) at PPARA −35,014 had a 5.7-fold relative increase in 3-year mortality (HR 5.7, CI 1.2-26.4; p=0.03), when compared to subjects homozygous for AA at PPARA −54,642 and CC at PPARA −35,014 (see FIG. 2).

### Example 3

### The PPARA −54,642 SNP Confers Differential Binding to PPARA Activators ERRα and ERRγ

Given that the ERR nuclear receptor transcription factors bind to single consensus half site sequences and are known activators of PPARA expression, experiments were performed to determine if ERRα and ERRγ were able to bind nucleotide sequences containing the PPARA −54,642 variants.

Electromobility shift assays were performed as described by Huss et al. (Mol. Cell. Biol. 2004, 24(20):9079-91) and Cresci et al., (J. Biol. Chem. 1999, 274(36):25668-74). Complementary oligonucleotides corresponding to the region of the PPARA promoter encompassing the SNP of interest were annealed to generate double-stranded fragments used in radiolabeling and cloning. The PPARA −54,642 G variant contained a C at −54,645 and a G at −54,642, and the PPARA −54,642 A variant contained a T at −54,645 and an A at −54,642. The positive control element consisted of the previously characterized ERR responsive element (Huss et al., 2004). Probes were synthesized by Klenow fill-in reaction with [α-³²P]dCTP using the double-stranded fragments. Binding reactions were performed essentially as described by Huss et al., 2004 using recombinant ERRα and ERRγ generated using TNT Quick Coupled T7 reticulocyte lysate (Promega) and 15,000 cpm probe per reaction. In competition assays cold competitor was added simultaneously with probe and protein. Gels were imaged on a Storm phosphorimager and band intensities were quantified using ImageQuant software (Molecular Dynamics).

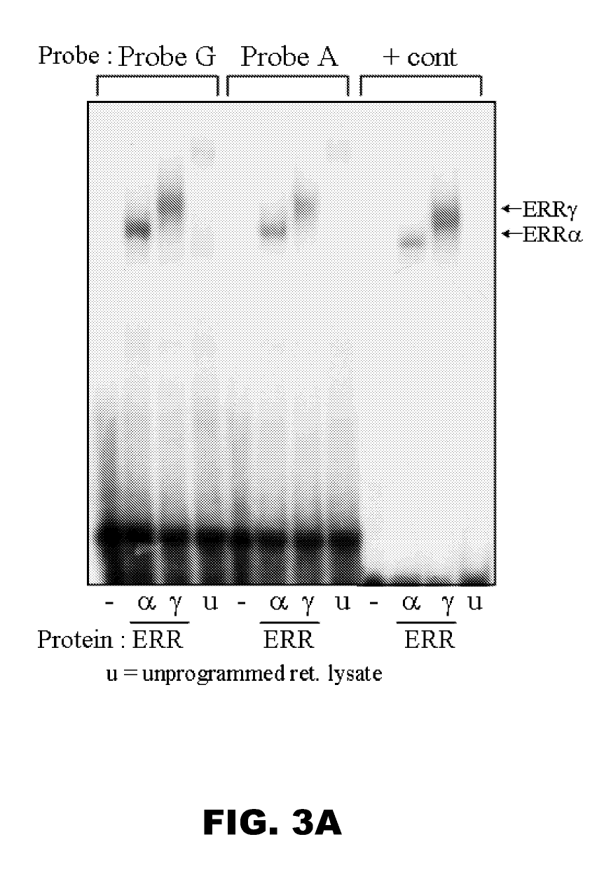

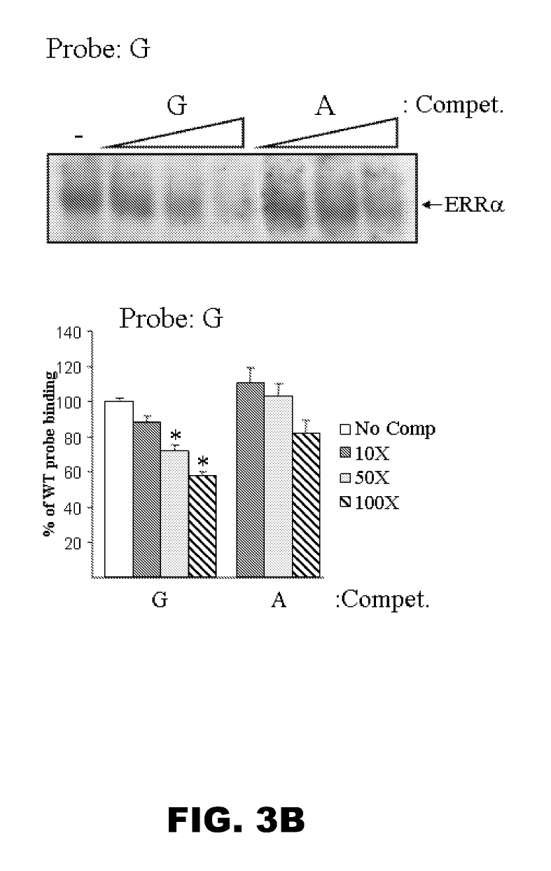

Two labeled probes, differing at the PPARA −54,642 SNP site (with the corresponding linked −54,645 SNP) were used in electrophoretic mobility shift assays to assess binding with recombinant ERRα and ERRγ proteins. ERRα and ERRγ bound the sequence corresponding to the PPARA −54,642 G allele with similar, or even greater, affinity than a previously characterized ERR responsive element (control; FIG. 3A). After determining that ERRα and ERRγ bound the putative regulatory element, it was determined whether the binding was affected by the PPARA −54,642 G>A polymorphism. The relative binding affinity of ERRα and ERRγ for the PPARA −54,642 A probe was qualitatively less under the same conditions (FIG. 3A). The relative affinity for the element variants was directly analyzed by performing assays using 10-fold, 50-fold and 100-fold excess of unlabeled G or A elements as a competitor in ERRα binding reactions with the G probe. As expected from the initial binding reactions, the A probe was less able to compete for binding to the G probe, and thus had significantly lower affinity for ERRα (FIGS. 3B and 3C) and ERRγ (data not shown). As seen in FIG. 3B, at 50-fold excess there was a 28% reduction in binding due to competition with unlabeled G probe vs. no reduction by unlabeled A probe (p<0.05) and at 100-fold excess there was a 42% reduction unlabeled G probe vs. 17% unlabeled A probe (p<0.05). Thus, the G>A substitution increases ERR affinity for the PPARA polymorphic nuclear receptor response element.

### Example 4

### The PPARA −54,642 SNP Confers Transcriptional Activation to PPARA Activators ERRα and ERRγ

To assess the functional relevance of differential ERR isoform binding affinity between the two PPARA −54,642 variants, the activities of heterologous PPARA polymorphic element reporter constructs containing 2-copies of either the G or the A variant element upstream of a TK luciferase reporter were assessed in conjunction with a vector expressing ERRα. To clone heterologous PPARA variant promoter-reporter constructs, (−54,642A)2c.TK.Luc and (−54,642G)2c.TK.Luc, double-stranded fragments corresponding to the probes used in Example 3 were ligated into the BamHI site of a luciferase expression vector immediately upstream of the thymidine kinase minimal promoter of the pGL2.TK.Luc reporter plasmid. Clones were screened by PCR and those carrying 2 element copies were verified by sequence analysis. Transient transfection studies were performed as previously described (Huss et al., 2004; Gulick et al., Proc. Natl. Acad. Sci. USA 1994, 91(23):11012-16). PPARA promoter-reporter constructs were cotransfected with empty expression vector or vector expressing ERRα and ERRγ. Transient transfections in CV1 cells were performed using Lipofectamine 2000 (Invitrogen) with 2.7 μg/ml reporter, 0.3 μg/ml each expression vector, and 0.3 μg/ml pRL-CMV to control for transfection efficiency. C2C12 myoblasts were transfected with 4 μg/ml reporter, 0.5 μg/ml each expression vector, and 0.5 μg/ml pRL-CMV using the calcium phosphate precipitation method. Firefly luciferase activity normalized to that of renilla luciferase was measured 48 h post-transfection on a Clarity luminescence microplate reader (BioTek). Four independent trials were performed in triplicate.

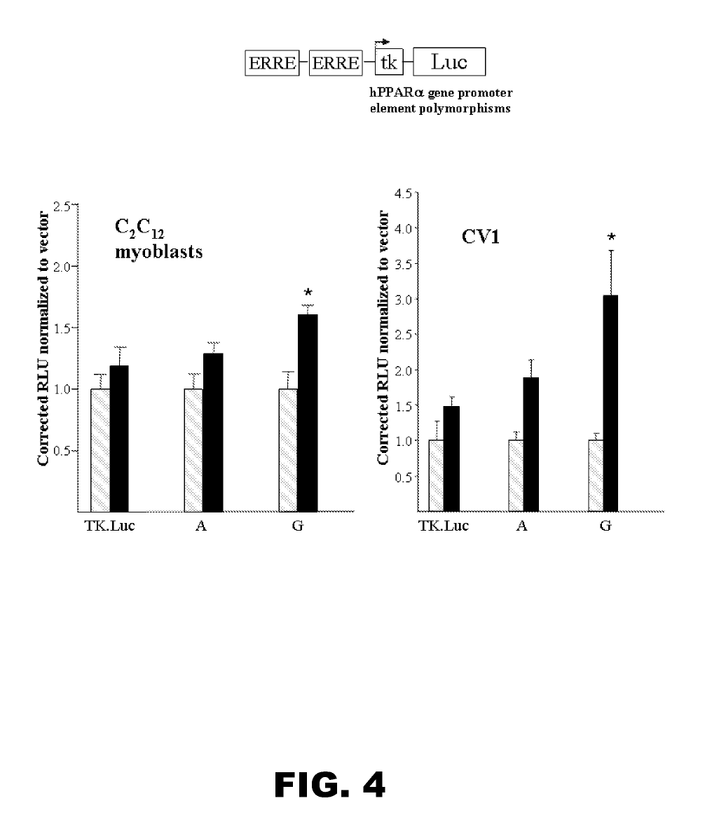

In C2C12 myoblast cells the G variant was significantly more responsive to ERRα-mediated activation compared to the A variant (FIG. 4). The same differential activation of the PPARA variants was also observed with ERRγ cotransfection (data not shown). Although ERRα-mediated activation of the G variant was significantly greater compared to the A variant (1.6-fold vs. 1.3-fold; p<0.05), the activation was modest. It was hypothesized that this was due to the presence of endogenous ERRα in C2C12 myoblast cells, moderating the ability of exogenous ERRα expression to further activate the PPARA variant constructs (thus decreasing apparent fold-activation). To test this hypothesis, similar experiments were performed in CV1 cells that were null for endogenous ERRα and ERRγ. As seen in FIG. 4, in CV1 cells the G variant had greater fold-responsiveness to ERRα-mediated activation compared to the A variant (3.1-fold vs. 1.9-fold; p<0.05). These results were similar with ERRγ (data not shown). Collectively, these studies reveal that the PPARA G variant confers a functional change on the PPARA promoter response element resulting in increased binding and transcriptional activation by ERR isoforms. This, in turn, is predicted to increase PPARA transcription and expression.

TABLE 1
Summary of PPARA Gene Sequences.

| SEQ | | |
|---|---|---|
| ID NO: | Name | Description |
| 1 | PPARA gene | (See sequence listing) |
| 2 | Promoter A of PPARA | Identical to residues 1-10,001 of SEQ |
| | gene | ID NO: 1 |
| 3 | −54,642 G variant | Identical to SEQ ID NO: 1, except |
| | | residue 3142 is G |
| 4 | −54,645 C variant | Identical to SEQ ID NO: 1, except |
| | | residue 3139 is C |
| 5 | −54,645 C and −54,642 | Identical to SEQ ID NO: 1, except |
| | G variant | residue 3139 is C and residue 3142 |
| | | is G |
| 6 | −35,014 A variant | Identical to SEQ ID NO: 1, except |
| | | residue 22,770 is A |
| 7 | −54,645 C, −54,642 G, | Identical to SEQ ID NO: 1, except |
| | and −35,014 A variant | residue 3139 is C, residue 3142 is |
| | | G, and residue 22,770 is A |

What is claimed is:

1. A method for identifying a subject at risk for increased mortality associated with acute coronary syndrome, the method comprising determining the identity of the nucleotide at position −54,642 of a nucleotide sequence comprising the nucleic acid sequence of SEQ ID NO:1 in a sample from a subject, wherein the presence of a G instead of an A at nucleotide −54,642 indicates a risk for increased mortality.

2. The method of claim 1, further comprising determining the identity of the nucleotide at position −54,645 of a nucleotide sequence comprising SEQ ID NO:1, wherein the presence of a C instead of a T at nucleotide −54,645 indicates a risk for increased mortality.

3. The method of claim 2, further comprising determining the identity of the nucleotide at position −35,014 of a nucleotide sequence comprising SEQ ID NO:1, wherein the presence of an A instead of a C at nucleotide −35,014 indicates a risk for increased mortality.

4. The method of claim 2, wherein the subject is diabetic.

5. The method of claim 2, wherein the subject is on hormone replacement therapy.

6. A method for treating acute coronary syndrome in a subject, the method comprising determining the identity of the nucleotide at position −54,642 of a nucleotide sequence comprising the nucleotide sequence of SEQ ID NO:1 in a sample from a subject, wherein if nucleotide −54,642 is a G, the subject is administered treatment to modulate the activity of PPARα.

7. The method of claim 6, wherein the treatment is designed to reduce the mortality from acute coronary syndrome.

8. The method of claim 6, wherein the treatment comprises administering a drug to modulate the activity of PPARα.

9. The method of claim 6, wherein the subject is a diabetic.

10. The method of claim 6, wherein the subject is administered treatment to decrease the activity of PPARα.

11. The method of claim 6, wherein the subject is administered treatment to increase the activity of PPARα.

12. An isolated nucleic acid comprising the nucleotide sequence of SEQ ID NO:3.

13. The isolated nucleic acid of claim 12, wherein nucleotide −54,645 is a C instead of a T.

14. The isolated nucleic acid of claim 12, wherein nucleotide −35,014 is an A instead of a C.

15. The isolated nucleic acid of claim 12, wherein nucleotide −54,645 is a C instead of a T and nucleotide −35,014 is an A instead of a C.

16. An isolated nucleic acid comprising at least ten contiguous nucleotides, including nucleotide −54,642, of a nucleotide sequence selected from the group consisting of SEQ ID NO:3, SEQ ID NO:5, and SEQ ID NO:7.

17. An array comprising a substrate having at least one address, wherein at least one address has disposed thereon a nucleic acid that can specifically bind to SEQ ID NO:3 or a portion thereof containing nucleotide −54,642.

18. The array of claim 17, wherein at least one address has disposed thereon a nucleic acid that can specifically bind to SEQ ID NO:4, or a portion thereof containing nucleotide −54,645.

19. The array of claim 17, wherein at least one address has disposed thereon a nucleic acid that can specifically bind to SEQ ID NO:5, or a portion thereof containing nucleotides −54,642 or −54,645.

20. The array of claim 17, wherein at least one address has disposed thereon a nucleic acid that can specifically bind to SEQ ID NO:6, or a portion thereof containing nucleotide −35,014.

21. The array of claim 17, wherein the substrate has no more than 500 addresses.


SEQUENCE LISTING (1 sequence)

SEQ ID NO: 1 - DNA, 113156 bp, Homo sapiens
  misc_feature (1)..(113156): PPARA gene
  MISC_FEATURE (3139)..(3139): Y = T or C
  misc_feature (3139)..(3139): Y is -54,645 upstream of translational start site (TSS)
  misc_feature (3142)..(3142): R = A or G
  misc_feature (3142)..(3142): R is -54,624 upstream of translational start site (TSS)
  misc_feature (22770)..(22770): M is -35,014 upstream of translational start site (TSS)
  MISC_FEATURE (22770)..(22770): M = A or C
  misc-feature (57784)..(57786): translational state site
aaacctccaggccaagtttcagggggaaaccaagaacctagagaagaacgctggggtcactggtgcctgcgagcatttgccaacctggcaagttgggctttgaccttggagggagtagaggggtagacagaggtcaaggtatgaggagcctgataggggcatatcaagctggaagcgtgaaaggcctcaccttgggtgaagactgaagcagagataaccctggcccctgccccaaaacaaggaattcgttagcattaaaaggagtgggaggaaaaaaggaaaggaaactcacggattcaagcacaaagagcctcggagctcctgcggacttggaccccttgcccatcacacccgggaggcccggagatggtggggcttctgtgcacttggttccaggtggtctgggctgtccatgtgaaagtaaacactatccttgtgagaaggaacctctgtccttggcctcacggaaaccccacagacacccttcaaagggccccaagacgcacacaaagatatccaagtctgaaagggaagaaggcaccatgagtaagaaccagcagacaagctagcggacacccgcgcggtctccccatattattagaaaattaccaaacaagacactagaggacatttcaaaccataatagaagagatggcccatgttcggtggccaagcatagcacagagctcctcactcctccaggaattccactgaagggtggcaagttctggagttcaagccactggggaggcaacaggagtgcaagtggccacagttcaggcgctgaggggcagaggcagggagggggcgctcccaagaagtccactgggagacgcaggcagggggcttcccaggcagcctcaccagcttcccttcacactgatcccctgtaaaggtgtgtccagcctcgatgtaggaggaacagggagaaactgatttgctattcataaaatgcgtctgcagaaacagattaactcctgatccactttcacggctgaagcagagcctgccttgtaaaacacatgggcattttcctatagttgtttttctgttttcagttttgtttgccatttcctgaatctaacaccttctgaggcggatgctaagagtttagaatttacataattacccgagtcacttgttaactccctgtaagcagcaccctcactggatttgatcacttttgctttgccatgcctgcataagcttgccaccgaaatactttaaaaaatggacttgaggccacacactgggctcatgccagtaatcccagtactttggatgtcaaggtgagaggatcactggaggctagaagttcaagaccaccctgggcaacataccaagatcccttctgcaaataataatacaaatagtaataataataataaatatatatatatatatatatatttttttttttttgagatggagtctcgctctgttgcccaggctggagtgcagtggtgccatctcggctcactgcaagctccgcctcccgggttcacgccattctcctgcctcagcctccccagcagctgggactacaggtgcacaccaccacgcctggctaatttttgtatttttagtacagatggggtttcactgtgttttagtcgatctcctgaccttgtgatctgcccgcctcagcctcccaaagtgctgggattacaggcgtgaaccaccgcgcccggccaataataatacttttaattagccagtcatggtggtgtacacctgtagtcccagctactcaggaggcagaggcaggagggttatgtgaacccagcagtttgaggctgcagtgaaccatgattgtgccactgtactccagcctgggtgacagagcaagatccaacctcccctccgcaccaggaaaaaaaaaaaaggaaagaaagaagacagacttgtgcctcacaagtggtacttttgagttcccactcaaactactgtctttcgagtagggcaagaccactttccattgtggtatctttgcagcagtgccattgtcccttatggaacagtgtctcaggaccccgctcctgctccttctgccggctctgagcaggtgaggacaggggactgggaaggtcccctgcatctgagcccgccggtgctcaagcatctttcttggctgcccaagaacattgtctgcaggacaatcccagctcctcagcctgcgtctctggccctccaggtgcccactgcttatgcttctcagtcacatccctggcctgcctgggctcagggctgactcttactccaagggtgggcagcatgaagctggagccattttgcagcaggaaggattctgcctactaggagccgaggccacacatgggggaaggcagggttcctctgccggagcccctcctggtgtgatgagggaggcaggcacacagagagggagttagaagaatggggttagtgggaaactgaggtctgccccaggcatgaggaggcagtgagggcagactcctagcccagcagggagaggaggtactgagagcacccaaccctgagtgaacagcagctagaccaggccgggcagatggccagtccctcctgcagcgcactccaaagtctcagggagccagggaacaggcagagctgagaaaacgtcctggaaactccacaggagcccagagcccaccatgttctctgagagaaagtcagcactgggcacgctggacagggaagcagggtcatgcttggaaaggccaactcagtccttgccacccactgagcacccactagctagttatttggttttgttttaaaaactcaactcatcacgaatactcaccaagatgctttgatctaatttcatgcctccacctggagagggggaacgatgaattgacagtctcccagcctgtcttccccaccagccatccaagaaccctgcctgccaggatgctggtcctgtgtctgcctgccccacgccaccgtcaccttgcagagaaacactgattgccttcacacatctggctccagccaactccctcctctgcacaccttgaggctcccaggcagagaggaggctcttccccaggccgggggcccccctgggagggtgtccaatgtcatgccagccaaggcccgtgtgggcgtagctcctgcaggttctcaaggttgtagyccrcaccctgctccttggctttgtgaaagatcaaaatcagggcctcacatagaactttatattccaacgttgcaagtctcaacacaaaccaatgtgttgacttgaaaataaatatcactaataaaatggagaagagagctggcatggaaagattgggaaaatgtcaagaattcttggtgctgtatgtttatcagtaaataaagcacatctgaggggtgaggggtcggcccttggaccttgggctcttacagatgggtgccattttcaacagctttggagacaaacatggtgtttgccaatatcttggttttgccaagtcaaagctaaccaatatcagcacttcatctaagcaattcccactaaggaaaggtattttcaacaacaacaaaagaatataaaggatatagtcttgggaaggaaaaggatatttgtcttgtttttctttgagaaaagaaatgagcctctcaatgttgcccaggctggagtgcagtggtgcaatcacagctcgttgtagccttgacctcccaggcccaagtgatcttcacacctcagcctcctgagtagctgggaccacaggcacgtgctaccatgcttggctaatttttttttttttttactttttgtagagacggggtctcattatgttgcccaggctggtctcaaactcctggcctcaagcaatcctcccgcctctgcctcccaaagtgctgggattacaggtgtgagccactatgcctggcctgaaaaaaaaggctattgctttttttttttctttttgagatggactctcactctcgttgcccaggctggagtgcaatggcgcaatcttggctcactgcaacctctgcctccagggttcaagcaattctgcctcagcctctcaagtagctgggattacaggcatgcaccaccacgcccagttaattttttgtatttagtagagacgggtttcaccatattggtcaggctggtcttgaactcctgtctcaggtgatccacttgcctcagcctccctaggtgctaggattacaggcccaccatgcccagcccaaggctctttcttttcttttcttttctttttttttgagacagagtctcactctgtcgcccaggctaatctccagtcactgcaacctccgcctcctgggttcaagcgattctcctgcctcagcccccagagtaggtgggactacaggcacgtgccaccatgcgcagctaatttttgtatttttagtagagatggggtttcaccatgttggccaggctagtctcgaactcctggcctcagatggtccacctgcctcggcctcccaaagtgctgggattacaggcatgagccaccacgcctggccccgaggtatttctttaaatcagctagttgaagttctataggaaactcttcacaatgtcagttttttgtttttttgttttgttttgttttgtttttttcactttgctgcagacccagaaaactttgtcaaacatgacctgtcccttccaggagatgagggaccatggcccaatcccacgttttagtgaaacccaagcagagcaatgcaatagggcagccccccttccactcagctgcacgggctccggttcagattgcctttcctggctggcaggatgctttggtgaaaggcagataatagggtgggacaggggatcacgggcgggagacacgtaggcactggggctggtgtctgcgaggcatgaggcctgcctgtcggctgtcctgagtggagagacaggaccgggcagtgggccgccataaagatggtgctggatgcgacaagagccgcatggaacacaggaggtggtggccacacaaggcagagggcagctttggcagggggacggttgccaagcaggcttcctgtaggaggcaactcttgacagagccctcaagctggtggttaccatgtcccaaatatgacccactcatcaggccccaccctgcaccacccccgtgctccctcctcaaactcacaccaggacccatccaggaaggcgtccctgccccattttagagatgagggggctcggatagcatcaccggctggaggttgtagtttgaggaaaaggtacaggaggatttgaatccaattctgttgaactccaaaccccaggcacccactcattccatgagggaaagttgggggacagtgcccacgtggcacggtgctgtgcacgtaagcttgcctgtcttgtgtgtttccaggctgtcatgccagacctcatctctgcacttgcagtcttccctggccgcagcagtgccactcactgccaaccactgggtcagatcattgcagaatccaactccacacagagcagattgtcctttatggaccaaatatgtctatctggttgggcgcagcggctcacgcctgtaatcccagcactttaggaggtcaaggcgagcggatcacctgaggtcaggagttcgaaaccagcctggtcaacatggtgaaaccctgtctgtatgaaaaatacaaaaaattagccgagtatggtggtaggtgcctgtaatcccagctactcaggaggatgagtcaggagaatcacttgaacctgggagatggaggttgcaatgagccgagatctcaccactgcactccagcaaggacataagtgcagagcaagactccatctcaaaaagagaagagaagagaagagaagagaagagaaaagagaagagaagagaagagaagagaagagaagagaagagaagagaagagaagagaagagaagagaagagaagggaagggaagagaagagaagaaaaaagaaaagaaaagtaaagaaagaaaatccgtctatctacacatcacactttgcagtgagactcccacggcacctagcatgacacagggaggggtcctgcagcttccagtccatgtcctggactggataaacaacatccatgatctagccctggagctggatcctgtgtgacgaggtggacacctgtccctttggaggggcaaagaagggaggcagtgcagctgcaggtgggctgccctgttcttctcaagatctacagagacagacatctgagccatgtcgaatgggccctgctgcaaagctggggactggttaattttcaaagggtacagtgtcatgacagcagcagtttctggacctgcggaagcttccagatcctgcaaagctagaatgggtgcacagcttaagcccctccccaccacctcagctgtgaccactggttaacatgtagcatgcagcacttggtgctaatcagatggagtcatgcctctgctgacaaccctccatggcaggtgtcccacagcccttaggaccaaagttaaactcctcactgggctgcaagacctgcctggccagggccttgcccagctctgcccgctttgtgccatctccacactctctccccttgaatgtctcctgccacaggcttctgcacatgctgctgccacccccctggagctgtggggccaggagcagccaccagggaaatctgatccttgaccctgggctcctgagtctgccagaggctgccgtatcagccaacagacagcttctgcaacagacaaccgaaccccgggggtacccagggccaggtgctgatggactgggactcgggagagttctcactccctgccggggaccttggccacatgctgccattcctggcccttcgttcctcatctgtaagatgagcataagcacagctgccttgtgggactcttgtagattctgtcccctagtatgacaatactttgttaccttggacccatggggttttgctgtcagtaccaggatggcaactaaggacatgcccatggttggccctcaccgacgaggtccagcctcgggtcgccaagcagccgcctgctcgctgccagtgagccctccctttctcagcccacggcccctgccccggtggacttccaccacctgacctgcaggcacgcttccaggagctcagaggccactgccctggcggatgacaccctggaagcacacactcccccttgacagagagctttgcacatcagcagagggcaagccagcagcaaacttcgcaggccctggctggcaaacagactccagcctggaccccaggcccataggagaagcttcaccctggcagggtcagggaaagcactggggtccctgaacacagatgaccgtcatcacctgctcccagcactcagggtctaactgctgccctgtactgagcggccaagccctatcactccaggttgcctcctccagcaaccccttcccgtctgaattcccagcttcccaattctttccctgaggctgtgggagagcctcggcttctctggctggctggcaggtcagcacttgcaggagagagcagctcagcagggcagacgtgggctttacagacctgccttccgtcctgcgccctgccgctggcctgggggacccagtgcagatggggcttctctgtgcctcagtctccatcttgcaatgtgggcacagtaataattaccctcagaacatcttccggatcagaaaatgtaaaaagggacctgagacagcattgtcaccacataaatggtagctatgaactaagatccatatgcaccagatgctcccagtagtcactaaggcgccagagaggaaactcaggtggtgctcagagacattttcaggagcactgatttgtcctcaaagttacagatttctactcagtccctgcgccagcggtgtttctgttctgtcgtgtggccctcatttcatcagcactgaagaaggtaaagagagacttcctggagctgggaatgggggtgagggtgtgccctttgtgacagttgctcccatgctgggtgtgtgacaaggtcttcgtggccaacagaaaattgcttcatctctctcatctgagcgtggtttcccagaagaaacctaccacccccgggaagcatggatatggggctgtttctggccaggatactcccaagagaagagtcctgaacttgcttctaccctcaggacgcctgcgggagactggctgacagcatttgattgaaaatgcttatttctgggacacatggatgaaaaacaagttttctctccctaaaaccttgggagccccaaaaaacccaagaatatcactttcctatgtctgctgagtgtacgtgaccactctgcacacccccacatagcctttgtccttgttcatgagaatgttcactaggtctttcaacacctggttgacctgggtggtgcatctgggtcacacacctgtgtggaccctggtccttgagagtctgggatatgggtatgccaggtaatgtctttgagcccggaggtcctcatgaggatgtagcccctgagctctgcgtgggcagcccagggtcagcttccctcaccaaacacaacaggatgacaagcttggatgtggctgcctgcacaccccacgagatatgcaggatattacgtgtacaggtcaccctataaactctgaaacaacataaatgatagctattgctggctaacatgtgcaagagaaggtgaggttgccgtgtgccagtgggaaggatatgtgggtgtctggagggtggggcaaagttcaccataggtgcctggctaatcaacaagggtgagtttaagagaagtttgtgaaagggccagttcccgcctgatctccagttcccctgcctaatgagatctgggtttgctttccggagtgggtcctcctgggcgcccgctgccacaaatagcacagtggcaggcacagctggcagcggagggcaggcagtggagcgggcatagcacacattgcgttcccgaggaggggagccctgatgtgccggcacccaggggctttgtgcattccgctccggcagctcgagcgtcacggcccgaacaaagcggctttgcagggcgctctcctactcctcgccattggccaccgggccgcgacctctccttgctctggcagagtcccagcgccttgcgtaggcacaaagtcagcccctcaccccccagcgcatgaagtaggggcgggcatggctcttgggccgctggagaggggtggggttgtggggtcggagctggcggcgcctccccgagcccgtccgcgttgcccttcaccctcctcgttcccccgcccaccacaccgccctggcacctcccgccacctgtttccttgtcctcccagctcgccttccccttctccttattttgcatcctggggttccagggacaaggtccctcccgggccgcctcccaccctacgcacttctgagcctcaagggcacccggtcccgggtcccgggtctagaccggctcatcgcacagagtagcagagccgggctcatcgaggaggcaggaggggctcgccagcgtggcacgggcgcccggcgggaacctccacccgccccgcggccgcgcgtccccgcctcgaattcagccccgccccggtgcgccgggctggaggggcgctgacgctcagcggtgtcccatcggtgaccttggacggtccctccacctctccggcctcagtttcccttggctgcagcggccgcggggcgctaggtgggagccgctgagcgctcccggggccccgcccaccgcgagcagccaatcgggcgccgccctccggggggtgtgtcccggggccgaggcccggggcccggagggcgcgcggggcgggcggggcttccgggtcgggcctcgggacactggctcgcgcggaccggggcagggggcgggccgaggggcggtgcgtgtcgcgggggcgcggctggcacggacgcgcggaggcggcgccgggcatgggccgtggacgcggcggccccgcggcgggggcagcgggcggcgggggcggaggcggccgctagcgccctgcccggcgccgcctccttcggcgttcgccccacggaccggcaggcggcggaccgcggcccaggtgcccgggggcgggcgggcgggcgggcgggaacgcgcgcgggggtccgcggtccgggcttcccaggtcccgggacccggagggcggcggacgggggaggggcaggggctgggcggcgcatgcgcggggcccggggtctcggggtctccgggtcccggggacccgggggcccggggtgcgcggctggggacctgagggcgaggagcgaggacacacaccgaggactcttgcgagggatctcggggcccagctcggcctccctcctagcgctgggggcctgcccggaacccgagtccgcggctgtccctggggtttggcgctgcgcggaggtcgggtctggggaccgcagcgactctgggtcttcgggttgtcccctcggagggagggcccacgggcggggacatcgggacttgccctttcctcggcgcagcggagctggggcgtcgccgactcagaaggtgctttccgagacctccagggatctccgaggcgaggaaacccgggccccggacagaccgaccctgggtgggtgcgcccggcttctgccgtcggacggagacgcgcgtgtttgttcctccagctgcgaccacctttgaggaacggttcccactttgtgccccaacgcggcggggcgaccccggacaggctgcgctgggccgggtggcttctctgcggaagccgcgccacgtcgctcccggtcggggccgctgagggtcgggcgcccaggtctttccggagtcccgggctgcgcggcccgcgtggtgcgggtgaagctggaggggcgcggggtggtgccagtggaagtcaggagggtcggccctgccccctcacgcaccccaaccgggcacaactgcacgcctgtgcttttctgaagtcttttttaaaagttaaaagagaggaagtgtgctccaagtgtcaggattctttccaagaaaaacccacagttgtccaatggcctgggcttcgtgggacctccggggctgcacgcccacgtcagcctcagccgacccctgccaggaaaccagggaggcccctcctctcccagcctccttgggataagggtgccttggggaactgggtcagggcaaggacacgggattttcctgggaaggaccctgcgacacccgtgtcgttgcggggcagggtcagcatgactttcctcttccaaggtgaagagttggggggcatccagagaacaaccgtaatcacttcctccttcaccttcttactgccaggctgaagctcagggccctgtctgctctgtggactcaacagtttgtggcaagacaagctcagaactgagaagctgtcaccacaggtaaatagaaggtttaatttactgtttccagatggaaatatttaagtgttttcagtgtttacttctgttgcactacagaccagcaatctgggggttattactttgtgatgcaaggttagatacgttttcagactgaaagtaaaatacatgtgcatggattcattttttttttttttttttttttttgagacggagtctcgctctgccgcccaggctggagtgcagtggcctaatctcagatcacagcaacctctgccactggggttcaagcgattctcttgcctcagcctcccgagtagctgggattacaggcgcctgccaccatgcccagctaatttttgtagttttagtagaggcggggtttcaccatcttggccaggctgatcttgaactcctgacctcatgatccacctgttcctcccaaagtgctgggattacagacgtgagccaccgtgcctggcctaggattcactttgaagttctgagttattgtgtgacttttgctaggaacttcattgcttcgtggcaggcatgttttgtataatttaaaacttgatgacattaactttgagaaacgtgagtgcttactagacccttgggatgtccacactgactggtaccgagtagtgtactgtctctgagctgttttcattttgatttgaatattaagcagatggcttcttgagatagacccgtgccagaacatgccagggataggctgaagaaacgggccagatgatacaaatttgtgtggtcaccatccatgagagaccagggacactggggctgatgatgacctctgcaactctgaagcaaaagtaaactaattggcaagttgggtgcggtggctcactcctgtaatcccagcactttggaagctggggtgggcagatcgcttgaggccaggagttcgagaccagcctggccaacatggtgaaaccttgtctctacaaaaaaatagaaatattgcctgggcatggtggcggacatctgtaatcccagctactcaagaaactgaggcaggagaatcgcttgagcctgggaggtgaaggttttagtgaactgagattgtgccactgcactgcagcctgggcgccagggcgagactccgtctcaaaaataaataaataaaataaaattaattaactaattgacattagaaaaaaatgttttttctttcttttcccacatcctttttttttttttttttttttttgtgacagagttttgctcttgtcacccaggctggagtgcagtggcatgatcttggctcaccgcaacgtccacctcacggattcgaacaatactcctgcctcagcctcccgagtagctgggattacaggcactcaccaccacacccggctaatttttgtatttttagtagaggtgggtttcaccatgttggctgggctggtctcaaactcctgacctcaggtgaaccgcctgccttggcctcccaaagggctgaggttacaggtgcgagccaccgcgccgggcccttttccgacatcttaaacgtaaagtaggagacgtgtcataatcatcgaatactgcagtggttttcattagctcctgtttgtcaaacttatgaacagagttttaaaaattgtgtatcagccgggtgcggtggctcacacctgtaatctttgggaggctgaggtgggcagatgacaagatcaggagtttgagaccagcctggccaatatggtgaaaccctgtctctactaaaaatacaaaaattagctgggcatggtggcgggtgcctatggtcccagctactcaggaggctgaagcaggagaatctcttgaacccgggaggtggaggttgcagtgagctgagatggcaccacagcaccccagcctgggtgacagagcaagactccgtttccaaaaaaaaaaaattgtatatgagagagacagaactagacagagaagaaggagaaaatgtgtcttctttatacactattttgtaacttgctttatcgagtaggttatgaaaaatcttcctatgtgaaaaacatttctgcatcatttgaaatgtctatataatatcccattgtgtttagatacaataatatttagccaatctctttatgtgtatatatttaatacagtcattctataaatattgactgagtagctgctgtgggctactgtccgcagtgctgaacaagacaagcatgaatccatgaaactgattttcataccagaatataaaaaagaaacttaaagataatcctcatcatggtaaaagatgaagaacctatttttgccgggacatcttactctttagtaattggtggccagtgttctttttcttgcatgctgttttggagagtctgttttttaaataaatatttaagtagcctgggcgcagtggctcacgcctatggtttcagcactttgtgaggccgaaggggatggattgcttgagcccagggcttcaagaccagcctgggcaacctggcgaaaccctgcatctactaaaaatacaaaaattagccaggtatagtggcgtgtgcctgtggccccatctacttgggaggctgaggtgggaggatcccttgagcctgagaagtggaggttgcagtgactgagatggcaccactacactccagcctgggtgacagagtgagacctggtctcaaaaaataaataaatatttatgtaatcatctttaagcagtgtttttaattttatttatttatttatttatttttgagacagggtctcactgtgtcacctaggctagagcacagctgcatgatcacggcctattgcagcctcgacctccctgggctcaggtgatcctcccacctcagcctcccaagcagctaggaccacaggcacacgccaccaggcctgactcatttttgtattttttgcagagacggggtcttgctatgttgttcagacctgtctcaaactcctgggctcaagccatcctcctgcctcggcctcccatagtgctgggactaagccatgaaccactgcacccggcataagtggtctttctttaaaaaaaaaaaaaaaaaaaaaaaaaaaaaaaccacattaattaaaatatgtatttgcttattataaatatatttgaaacatgccaatttttcttctctttttttgctctattggtttctgtgtgtggatggatatatttttaatggcaaataggatgagtgtctttacttccaagtagtcagtgtttttctttaatgtttgtactaattttgtcacattgcagttagaggttgtggcctgtctaatttctgcttttttggaacttgagagtctctgtttttatttgtttttggtagcctggcatagagtccatttttcttttcttttctttttttgagacggagtttagctcttgttgcccagactggcgtgcagtggcgcaatctcagctcactgcaacctccgcctcctgggttcaagcgattctcctgcctcagtctccccagtagctgggattacaggtgcccaccaccacacctggctattttttgtatttttagtagagacggggttttgccatgttggccaggctggtctcgaactcctgatctcaggtgatccacccgcctcggcctcccaaagtgctgggattacaggtgtgagccactgcgcccagctgtagatactttttaaaaaggtatagtttctgattatggggtagaaatgtgctatgtctgtcatttcagccttatgaattgcccagaataagctagatcacctttaaggccatgtggttagggaaacttgggcacagaatttacattttcaacttggtgataagatgggtttaaggtaagaatcaaataggagaaagccttagctgttccagcggcccatgtttaaaagaatgtgcttctttttccaagtatttctgccgcttgcatgcactgagcttctttggaaaggagcaccatgcaggcatattttccagacaggaccggatttgctcgttactcagaggtgtgtgcattctttgcttttaggatatttaattagcatcttttaatagtgatattacggtgtcttaaaagtttatgcatttgaaaagaaaagaacttactccttgccaggtctcaacctatcatggttatctttgcagctgagctgcgttggttttgaggctcacatatggtaaaagtggttggaaatctggaaatattgctgtgtatctgcaaagcagcttgatatagtggaaaaggtattaggtcattaatcatgagatttggattctagccccttagctgctgcctgccaggcctggagacctttgttctcttctttaaactgctgctttctcatcagaaaatgaagttcctctccataccacctctctgaagggctgtgaagctcgaagtggcagcttaaaaaactgcccatctcaggaggtgtcttaagaaggaggacataccgctggctcctgcctttctcacttagccaggtctgatacctgtgttgttttcactgtggccattttaggatttttcaaaggctttcagaaagcaacatgctaccgtaccccttatacaccaaaactggttttcattttggaatataaaagtgagatttctccaccagtacaataaagttgttacaagtggttcctatgtgtttgtttttgtttttgagacagagtctcactctgtcacccaggctgcagtgcagtggcacaatcttggctcactgcaacctccgcctcccgggttcaagcaattctcccacctcagcctcctaagtagctgggactacaggcacccgccaccacgcccagctaatttttgtatttttagtagagatggagtttcaccatgttggccaggctggttttgatcttctgacctcaggtgatccacccgcttcagcctcccaaagtcttaggattacaggcgtgagccaccacacccggcctcctgtgtgttttgaaggcgattgtgacctcaggttttggcagggctataccttgtgtttgctcttactccaactccatggcatacctggaccaggcctcttcatcttgaagagggatctgctgaaatgcaggcccagtgaatctccccatgcctggacacagttccgtcaagccaggacccggtgctgcctgcacccctgtttctgttagtctgactgtcctcactgagtctaactccttgagggcagagaggatgtcttatttatttctgccccgctagccgtgtaaactgagtaggtacttgtaaatgttcattgaataagtacctgattaatagaatttaattcaagaagaatgtattgatgggcctgtgtggtcaccacagtactgagatgtaggtgggagctggctgaagggggaggcacctaaacaggagcgcagacagcggcacctacggatgatggcccgctccatcccaccgcagcgaaattgtcccagacctctgcagctccccccacacctagactgagagagagctcttcttccttctgtagggagcaggtgtttcctccagatgtccaatatgtacctcccattacagcggtgttaggaaggtgagggctgccgctgaaagggtccccttcataatcatcactagatttggggtatattatggattaaatagaatttttataagatgacctggggatctatttaaataaaatcctctttctttctgcaagatcatggatttaaattcaacacaactgacttcatagggaaggggtatggtgaaagggaagtgaggtgggcagcactgatatttaacaaggtgagggtccttctcctgctgtgactgtcacattaaaatattcccaggagaaattggagaaaactcagatgaaatatcgtctgtgttccaggaggcaggactcatcggaatgcttttattttgctccattttaagagatttgcagataaagaggagtgaagatttctattcagatttacttgctttatacttttaacttatagaccacaagccaactttcgaaagagcatcattttgaatagtaagagttaggaaggcaaatacagaaggactaatggcttccaagattatgagcttcataggaatggtttgagatgaggctatagtaaagcagaatattgaagttcccccaccccctttcatttttcatttttcatttttaagagtgagcgaggccaggcgtggaggctcatacctgtaatcccagcactttgggaggccgaggtgggcagatcacaaggtcaggagtttgagaccagcctggccatcatggtgaaaccctgtctctactaaatgtacaaaaattagccaggcttggtatcaggtgcctgtaatcccagctactcaggaggctgaggcaggaaaattgcttgaacccaggagtcggaggttgcagtgagctgagatcgcaccactgcacatctcagaaaaaaaaagagtgaggccccaagtttttttgcatttgtttgtaactgaatacgtctgaagttatgtgataaccacgccaaggtgacaaattgccaagtttcagtaaaagagacccagttatttagaggttgacacgtggatatgtccctttctaagaagttcgtggtcagctttacatgagtatttaaatgcgtgtttataattcagcaatatggcttgtaaaatacagattgccaatcaagtgacatgcaaatcttgatgatctgaaacaagttttcttctgttatctatggaagaaatggtaatagggatatttaagtgggatgaattttttgaagcattttcaggcagttttccacatggaacaaaataacattgagtgggctgctaacatgaggaacatattgccctctgcctaggattatgagtaaatttgataaattctagactgcagtctcattttagctcattttatgaggcagcttgacaactgggatagtgtctcttttttttgtcgggggtgttgaggctggagtctcgctctgctgcccaggctggagtgcactggcgtgatctcggctcactgcaacctctgcctccggggttccagtggttctcctacctcagcctcctgagaagctgggattgtaggcatgtgccaccacgcccggctaatttttgtattttttagtggagacgggctttcaccatgttggccaggctgggtctcaaactcctgacctgaagtgatctgcccgcctcagccaccctaagtactgggattacaggcatgagccaccacacctggcttctgtttttctatctgtgcattggggatgaaattaacacaaatgatgtttaaagaaaaaaatgctcagagaagttagaaatgtgctttaaattggaatcatctcttagtatgtaaaagttttttgtaatagaaacaagcagggcagtatttgacctgttgacagtgtccttggactttacaatttgtgaagcagcgtattttgcttgagttgtacgattgtcgtttttttccctccactttgacaactgttacagaacctgtcaccagatacaggcaagggaggttgggcttcccatctctgcacggcttccctgtgattcacaagcaagcaatcagaagtgcacaaaagtttagaacgcgattttcattctcttctttccttagaaaaactcgctttgttagccttttccagaaaggaaggcactcaattgttgtaatactcaaatcataaaaagaagcctagtctagtctattcagcaaggtgttctgaaagagggaattttttaagttcaattatgcgaagatcttgaaggtgggactcaaaggagagggctatcctgggaagaaggctttggaaaatgagaggcatgaaggggagagggtatttaaatgtgtttgaagccaaggatccttgagagaaaaagctggcactaacagcgttcaaagaacttgcgtgacaagtgatgactaatgacactgagggtgggttgtgggtgcctagtgaattcctccgaagccaagagagaggtttccagacccagggaagaaggtgtgtacacccagaagtagtgtagggacagagattccgatcacaagctgtgactggaagacgccgaccaccactgcagcagcctgaaaaccacagtcttgaaccgccagcgaagggctgggaagtgcggatccagggctggtgcactgaacccagaggagcaggctcccattcccagccaagggtggcagctggcggggatctttccagcagaaagctgtaagtggaagctttcaattcagagcagtagcaatgccttcaaagtcccaggcttcacgtgggaacagagaatgtgaagagtatttagcaggatgccaatataagaaatctatattggtgttcgtttgtttgtttttgagatggagtctcgctctgtcacccaggccggagtgcagtggtgcgatctcagctcactgcaatctctgcctcctgggttcaagcgattctcctgcctcagcctcctacatagctggtactacaggcacgcgccaccatgcctggctaaatttttgtatttttagtagagatggggtttcaccacgttggccaggctggtctcaaactcccggcctcatgatccgccctctgcagcctcccaaagtgctgggattacaggcgtgagccaccgcacctggcccaatattgtttgtttatttatttcttgacaggatctcactctgtcaccaggctggagtgcagtggtgtgatctcagctcactgcaacctccacctctctggctcaagcaatcctcccacctcagcctcctgagcagctgggactacaggtgcacaccaccacacccaactagattttgtgttttttgtagagatggggtttagccatgttcagctagtctcaaactcctgggctcaagtgatctgtccgccttggcctcccaaagtgttgggattacaggtgtgattcatgatgtccagcccagtatttttctttcactctggaaaccaaaaattattggctttttttcctgttgcattccctttacttagatgaatctagcaaggttggctgttagtgtctaggtcagaagtctaagtgaaagtgaatatttaaccacactcaagcacagctgatgatctttaatactaatagaggtataagacttaaaagaaacaagaacccagagggaaaatatggccatggactcagagaaaaccacggcagcttccatggactcataaaaagagctcaaaacctaggaagtggatggagactctttttggaatgaatgaattcaaatgtgggctttcttagtagattaaatcattttctagaaggaatttcggaaggatgtgtgcccaattatggtatcaggtctgttgtagactcttcaaggaggaagcctctgaaagacaagaaggaacaattaaaaattagaattcaggtgagtggatcacgaggtcaagagatcgagaccagcctggccaacatggtgaaaccccgtctctactaaaaatacaaaatttagctgggcatggtatggctgtagtcccagctactcgggaggctgaggcaggagaatcacttgaacccgggaggcggaggctgcagtgaaccaagattgtgccactgcactccagcctggcaacagcgagactccatctcaaaaataataagtaaataaataaataaataaaaattagaactcagaaaaggaattaatttcttctgagagagaaaaagatgagattctagcctaaggtgtaacacatccatccaccaggtatcatttttatacacgtgaagttaaatcaccaaaggaccaggtgagcagatgtggactttccgactgtgtgtgtgcgacttcctcagagccctcagtggcgttcccttttccgcgctagcgtttggtccctgcgcttttctggatgcccccaccccctctggctccacgaggccccctgtacgtcaccatcacctttgtgagcttgaaacctgtcacccacccgccttccagatgtcacctgggccctcccggaggccctcgccctcagtgtgtctgattctgagctgtcctgcgttttcccctcccctcaccctggcgacccttttcggtctcagttgccagcctcctgctagggctgggtgggggacatcaaaggcaggacaaggtgtagggtcctcacccaccacttagcagctctcagatgcagacagatttttcagctggcctgtggctcagtttccctcagctacaagaggggtgcatgctagggtttctctggattgctgcacctggcaggtagtgtgagcttggtaggtgcttccttgcttatcattgcctctcccatcttaatgttgtcccatccatccaatgtttatgggatgagaggttgataggagggcatggccctgacattccagggactgaccgacacgctgtctacacaaaccccttctggttcttctcgtgcactgggcgtgccggagacacactcccttaccctcataccccgccgcacccctgtgacctctacctttgagacctcagcttaaactcactcttagggaaggctccctgaaccacctgctggggttgtatgctgatgcaggtacttgtaacacctggtgcttttcctttcgtgcactcaggcagtgtttattcaagtgatggcttggtgacagtggctctccctgagaccccgtgaggccagagtccttggctcatcactcatggttgaacccggagcctcttgctggtaggtgcttcaggactggctctgggagcctgtggctcctgccgggtacccaccggttgagatacctcaagttttaaatgccacctccttcctgaagccttccttgctgctcccccaaactagaggcaggagttttgtccttcagataacctatggcatttgagtcactctgatttgatgaattctgccttcacttgagcagctattaggggcatatgtcagtcattcattcctcagttcatgtatttattcagcaaatatttactgagcacgtactgcgtgccaggcactgtcctgctgtggaaaacagcaggcatgattccctgccactaccaaccactgcatcgcataactggcagactcccagcttcaaggagaggcacggagggaaactgagagcagcctgcagaggggaaagagcggggacagagggtcacggaggtcgcaggggcgtgtgtgcagcacctgccagtgaacggaatgtgcggctccagatgtcgttgtctttaaacttcggaatttcctttcactaaagaaccaagtccagggggaggaaagagtgaatacaaattatccaagaaactcaagagctcattttagttctcctgattatgatcttaaaggcattaagcgctcaagttaaactccttgtgacccacataggttagcagaatttaaatcctaggtgattmttaactctaatcatacatctaatgacctatattgaagatacactgcctgcttagttgtggcttcagcctttgctccgtcactgatagttctagcctgaaaagcaaatgagccctcatgctcacgatttcaccacagtcacataagcgggaagagcaggctcctggctgtggcgagcttgactccatttggtttgatagaaatgagaggtagatgattccctagacaaatgcaggcctttctcgaagcccctttcccaggacgacgtgacatgagtggtctgtgccttccagggcagccacgtcatgctttgcccagccagggcggtggggagggagacagccacatcctgcccggggctcctgggccccgctgcatcaagtgaaagcagggctggctccctgatgtccttggagaagtcgcccacactgctttcccccatgggagtgacaaggatgtgtcccgccagccttccacgacggaccccccactctctattaattcccaagaaaccaggccatggaggtgggtttgagggtttgtattggtgttttttaaagtcaggttgaccgagtgcggtggctcacgcttgtaatcccagcactttgggaggctgaggcgggcggatcacatgaggtcaggagttcaagaccagcctggccaacatggtgaaaccttgtctctactaaaactacaaaaaaaattacctgggcgtggtggtgggcgcctgtaatcccagctactcaggaggctgaggcaggagaaacccttgaactagggaggctgcagtgagccgagatcgcgccactccagcctgggtgacaagagtgagattctgcctcaaaataaataaagtcgggtttattaagatataatttacatacagtaattttttttttttttgagacagagtttcactcttgttgcccaggctggagtgcaatggcacgatctcagctcactgcaacctccgcttccagggttcaagccattctactgcctcagcctcctgagtagctgagattacaggtgtccaccaccatgccttgctaatttttgtatttttagtagagacaggttttcgctatgttggccaagctggtcttgaactcctgacctcaggtgatccgccagcctcggcctcccaaagtgctgggattacaggcatgagccactgcacccggccagtacatgctttcttgatttgtctgtttcccacctgtctcccctccctagaatggcagctccatgacgacagaggtgtttctctgttttctccatggctgcaccctcagctgctagaaggtggcccagcataggaggtatttaatgaagccttcctctccacttaaatctacacccttgtgcttattaaaaggtgacagttttctgtttgaaaattttattagtgttttaatgagaaagttattatttgggtaatgcctgaatatgaggaaaacattaagggtagaaatgtaattgttttcctatttcattcagtctatggattttattgaagattacagaattacttctttgtagctatggaagtaaaaaaataataagacgagtagctatttcaaaacgtagggctgataaatttgggatggtttgagaacgttaagttggggaactccatttctttttttacatttttatttattttcatttgtttatttatttatttgagacagagtttcgctctgttgcccaggctagagtgcaatgccatgatctcggctcactgcaacctctgcctccggggtataagtgattctcccatatcagcctcccgaggagctgggactacaggcgcctgccaccacacctggctaatttttgtatttttagtagagatgggatttggccatgtcagccaggctggcctcaaactcctgaccgcaggtgatccgcctgcccttggcctcccaaagtgctgggattacaggtgtgagccaccgcgcccagccagggaactgcatttctgacagtggctcagtagtttggaagttaactggcaaaggtggacagaatctttaaacatatgtggaggaattggagagtttacaagatagtgaagaactgccaggccatggtctggagaagatggaaacttgatgtttggggccattgtgtccctggggtgttggccaatttatgaaagaagcagttaagagcctgagtggcacttttgaggggctagaagggaagaccctggtaaacatcccaaactttggattgggacccaaaaaagctccatcccaggagtacaggtgacctggaaacggatcagcgtaatcgaggactgaagtccagttctagctacgcccagtccttgagactggattaaggtgatctcagattgcaaggacctcaaatgcctggcagaagcaagtgaatatccttctggaggaacagagcctcatcctaggcctctaattatttttaaggacaatttttcaaatgcaggctttcctccctttgcacagttcccttatgcataaatttcagtcagtggccagctgcagtggctcatgcatgtaatcccagcgctttgggaggccaaggcgggtgaattgcttgagtctgggagttggagaccggcctgggcaacatagaaccccatctctatttttaaaaataaaatattaattatcactgcttagttaaattatagtggtctcccaacaatacagatcagatcccagctcccatggtatatacactgtgagtgctgtataaagtacaagctctgccgccagttctccagcctacaaatcacagtatagataacagatgtgcatgatgatcactggccaattgcgtcacttctctcaaagtcagtctgtgattggtccctgagcatctgtcggtcagtttcatgcacagactgcaaagcatatggttttgtctactctttgtctctcagtgataaacccacatggcattttgtaaaagtggatacatcaggccaggtgtggtggctcatgcctgtaatcccagcactttgggaggctgaggcaggtggatcatttggggtcaggagtttgagaccagcctggccaacatggtgaaaccccatctctactaaaaatacaaaaattagctggatgtggtggcaggcgcctgtaatcccagttactggggaggctgaggcaggagaattgcttgaacccaggaggcagagcttgcagtgagccgagatcatgccactgcactctagcctgggtgacagagcaagactaccatctcaaaaaaaaaaaaacaaaaaacagtaatcaagcatgaaaattatgaaatgctcagagataaaatgcgcgaggcctgtacactgtaatctacaaaacactgctgagagaaattttaaaagacctaaataaatggcaagttataacatgctcttgaatcagaagactcagtatcttaggatggcgacttttcccaaaatgatctacagattcaaagcaatcggaatcagacctcagcatgcctacttgtagaatttgataacctgattctaaagtttatatggaaatgcaaggaacccagagttgctaaaataactttgaaaaagaacaacacagttgaaggacttagactacatgatttcaagaattattataaagctacagtaatcaagacagtatggtattgatatgaaaatagaccattagatgaatggaacagaatagcaagtccagaaatagatccacacatatatggtcaattgattttcagcaaagtgccaagtcatttaagtggggaaaagataatcttttcaacaaatgataccggaacaactggatagccatatgcaaaagaacctcaaccttcagctcacagcactacaaactcataattattatcattatattatactattatgtaataatagtatatatcatgttacatattatattatgtaatatatattatatgatactgttatgtcatataattattattgaaatgggtcatagatctaattgtaagagttaaaaccatccaggtacagtggctcatgcctgtcatcttgcactttgagaggccaaggcgggtggatcacttggccccaggagttacaagaccatcctgggcaacatagcgaaacaccgtctctacaaaaaaatgaaaaaattagttgagcatgatgacactcacctgtagtcccagctgcacagtagtctgaggtgggaggatcacctgagcccagagaggtcaaggttgcagtgagccatgattgcaccactgcactccagactgggtgacagagagaccgtgtgttaaaaaaagagttaaaactataaaaccttcagaagaaaacatatgagaaaattctagtgatttggggtttggcaaagattccttgaacatgatttaaaaagcattaactaggccaggtatgctggcttacacctgtcattccaatgctttgggggaccgaggtgagaggatagcttgaggccaggagtccgagagcagcctgggcaacataacaagagtgggtctttaccaaaaaaaaaaaataaaaagcctgtgccaggcacagtggcacatgtctgtagtcctagctactcacgaagctgaggcaggaggatcacttgagcccaggagttgaagcttgcagtgaattatgaccatgccactgcactccagcctgggccacagagtaagactaagactcagtctcttaaagaagaaagcgaccgggcgcagtggctcacgcctgtaatcccagcactttgggaggctgaagcaggtggatcacaagggcaggagatgaagaccatcctggctaacacggtgaaaccccatctctactaaaaatacaaaaaattagccggacgtggtggtaggcgcctgtagtcctagctactcgggaggctgaggcaggagaatggcgtgaacctgggaggcggagcttgcagtgagccaagatcgtaccactgcactccagcctggacaacagagcgagactccatctcaaaaaaaaaaaaaaaaaaaaaaaaaaaagaagaaagcataaactataaaagaaaaaattaataaattagtcatcctcaaaattagaaacttttactcatcagaaaacacttaataaaatgaaaagtcaagccatagacttagagaaaatatttacaaaacatatatctgacaaaggacttggatatggattatataaagaactattgtaattcaataagatgtcaaacaacccaattaaaaatgggtgaaagatgaactaactcttcaacaatgggcatgtcatttgaatggatggtaagcaagcacatgaaaagatgttcatgtgcctttccctcattagtcactagggaaatgcaagttcatagacatctctcttggtagaaagatatcactacacacccacaagagtggctgtaattaagcagtctgaccaagtatgcgtaagaatgtggaataagaactctcatacactgctgatgggaatgtaaaatgatagccactttggaaaacattttggcaaataataccacttacattattatcgaaaatattgtatacctgaaagaactcaaagtgaaaaagctatatactgtctgcttccaaggctacacattatgggaaaggcaaaactatgaagacagtaaaaagatgcgccagtggttgccaggggctcatggggagggaaagaggaatgaataggtggaacacagggcatgtttagggcagtgaaactattctgtatggtaccgtaacgatgaatacatgttattaggcatttgtcaatacccataaaatgtacaacacaaagagtgaaaatgaaaactgtgggcttcagttagcaataatatgtcaacattggctcatcagtggcaacaaatgtacctcaccaatgcaagatgtttgtttgttgtttgtttgttttgtgacggagggggtgcagtggcgcaatctcggctcactgcaagctccgcctcccgggttcacgccaatctcctgcttcagcctccggagtagctgggactacaggcgcccgccaccacgcccggctaattttttgtatttttagtagagacggggtttcaccatgctagccaggatggtcttgatctcctgctgtcgtgatctgcccgcctcggcctcccaaagtgctgggattacaggcatgagccatcacgcccggccaccaatgcaagatgttaataacagggaaactgtggtgggagtgaggtggtatatgagacctctctgtactttccactcaatttttctgtaagcccaaaacttctctaaataagaaagtttattaattaaaagttacttttatagtgtatctatatctaggaataaatctgaaaaagatatataagatctctactcagaaaactgattatgttattaagagagcttaaatatagcccaaataaatagatggatatactatgttcatggaagggacagctcagtattaggaaggtgtcagtcatcttaagaaaagcctcatgtgtcacacaagggatactgacatctgacaccaagcacatgtaggcatcctgactacgtttacttgaatgatgtggactttacagagctgactatagacagttcaaatggcctgaaaactgttcaatgcactccctcccaggctgtcatgggatgcacttcaggaactttactttttaacaagaaaattcagttttcctcttaaacagctggcttctgttccattagcattcttgtcactttaagttgcattcatctttgttttttttttttagaaaaacatttgttctgcaaccagtcttgtcctttaaatacttgtactgtatacaggctctttttcataggtccattacttaaaatgatgtaagtgtgtttttggtggcaggggggtgggagttgtttgttttgttttgttgagacacggtcttactctgtcacccaggctggagtgcagtggtgtgatcttggctcactcctggcctcaagtgatccacccacctcagcctcctaagtagctgggaccacaggtgtgtaccaccacacccagctaattttttttttttttttttttttttttgtagggacggggttttgtcatatcacccaggctggtctcaaactcctggactcaagggatcagcctgtctcagcctcccaaagtgctgggattacaggtgtgagccactgcaccggtcctgatttgagtttttgtaagacagggaacaatgttcagaatttagcaccaatgtcagactcattctgtaaatttttattgaacgtctgcctggtgtaggagaggaagatgacagacaagaattcttcctccaagagttacaggtcagttgagcagaaaaggcatacatcaatacccacaatgagagttgtcgtgattcagaggagggacaaagtccttcccctggagggatcctgagcactttggagaggaaaggcatctgtactgccccccaaatgtgtagaatgggatgcattcctggcagaaagaagtaggataaagtacagaggccagggctgggtgcagtggttcacgcctgtaatcccagcactttgggaggccgagacagcagatcacctgaggtcaggagttcgagaccagcctggtcaacatggcaaaaccctctctctactaaaaatacaaaaattagccaggcacaatggcaggtacctgtaatcccagctacttgggaggctgaggcaggagaattgcttgagcccaggaggcagagatcgcagtgagccaagactgcgccactgcactccagcctgggcaacagagcaagactctgtctcataaaaaaagaaaaaaaaaaagtacagagtccaggaagcctggggtggggctggcagatgccgagtcatctattttggccagagttcaaggcttgctaggggacatgaagagaagattcgtgcattctagttcaaactccaccagatatttgagctccttctctgtaccaggcattgttctaagatacgtaagtgaacaaaacccatgacaccctcgtctatgagagctgatcctctggcagggacagacaggtcatgagtggagtgatggagcagctggcctggtgacttagccgccttcaggtacagtaggaggagcaagcccaggacaggtgagtgggtcaagggtgccagaaggggtgagggcaccaggaagctggtccagtttggcttccctgaggtggtgaccaggacctagcatctgaggaagggctggaagcaggtgagagcaggtggagcagacatcaggatgggagcatcctgacagggagggcagcagggtgggctcatgagaggaacagccaggaagtgtgactcgagcagtgtcctggagaggaggaggaggagaaagaggtcaggaggtcccaggggagaggcaggaccagtctcgtggaggtcggggccgttgtgaggactctggtttgtgttgtgtgtgaaaggccatgggatggggaccagcgagggcttcttaggggactggatatgctctgatctagctgctaaaaagcccccttgggcagcttgcagggcccgggcagaagctataggtggttctgaggtttgcagaggggcctgaaggggtggggcccggccaagcaaggtggctaagtgggaaaggctccaccgcgttgggtgtaggaagaccttgaccttagctccagcccagccactgagcagccgtgtcgccttgggtgatacctgtccctggtcggtttccctacctgtgaatctgggtacttggaagccatgctcgagaagagcccatccccaggaggtgatcagggttctcctccaggtgaggaacctggcagccgtgtgtgagaaccttagaaaagggagagggaagaggctgtggcaggaagtgaggagggagttagtgataccctgggcaggatgccatgagctgggatggaaaccacaggatgaatgcaagtaattaaaaaaaaaaaaaaaaaaaacagcattgggccgggcagtggctcacgcctgtaatcccagcattttgggaggccgaggtaggtggatcacctgaagtcagcagttggagaccagcctagccaacatggtgaaactgaaaatgcaaaaattagccaggcatggtggcgtgtgactatagttccagctactcaggaggctgagacaggagaatcacttgaacctgggaggtggatgttgctgtgagctgagatcgtgccactgcattgcagcctcggtgagagagcaaggccccatctcaaaaaagaaaaaaacagactttccgaccaaacgatcgacaaaccagactgtccaaacagccataagccgtaactttgtgcggaggtaaaagaccgaggtcacatcgggacctgttggattcaaggcatgttgacagctgtttccaggcttcagatagagcctccagctggcagggtggccacagggcttgttgagtaggaagcctcgttgctttgacaggttacttggccccatgagggacaatcccatagtcagttacccagaaacgtgactgtctccttgaaatcctcagcatggggtcttatgaataaacccttactagatttcctgttctgtcttatttttatgcagagctttactttatagcagaaaattccatttttacccttaaatggcttgcttctgctcccttagtgttcttgtcactttaagttgcattcatctttgtccctttagaaaaggatttgtcctgcaaccagctcttgcagaaggtacttggtttattgttaaccgatgtttgctaaatgtttgaattatgttgagttgcttaaagtcatgctatcgggtagatgttgtggctgttcttttcactctcttatttggggatttacaaaacagttatgtttttagttttcttttatttgttgtgttgaataggaatgtagctctgggaacctctagttccaaataagaaagccttggacacatttccagttggcaagctggcaaaatgaagggcgtacaagttgttagagaggctgggagcctatttaagcacccagcttcaggatgggacatgggatatacctcgagttagaggttcttattaactgtggattcttctatgcagatatctgtcacaatataagttactataagtcagtactaaggcagctgctacattctgtttgccaaggggaagaagaaagcttggaaatggtattccttaaaaatgtcagtatcataaaagacaaagaaaagctgcggaaatgtttcagattaaaagagagaagacaataaaatgtaatacctgactctgaacagcatccagtactgaaggaggaaaaatgctatcaaggacattattgggtcaattaacaaaatttgaatacgaatcatagattgaactgtatctgttaaattaacagaagcgaagtgttctgtggtgtgtaggagcacactgccattcttagcaaacgtgtagtttagtatttaggagaaagggccatgaggcatgcaactcaccctcaaatacacacacacatacacatatatacatacatacctataaagaaagaaattatgggctaggtgcagtggctcatgcctgtaatcccagcactttgggaggccgaggtgggtggattgtgaggtcaggagatcgagaccctctctactaaaatacaaagaattagctgggcgtggtggtgcacgtctgtagtcccagctactcgggaggctgaggcaggagaattgcttgaacccaggaggcagaggttacagtgagccgagattgcaccactgcactgcagcctggcaacagagcaagactctgtcttgaaagaaggaagaaagagagagagagagagagagagagagagggagaaagaaagagagagagaaagaaagaaagaaggaaggaaggaaggaaggaaggaaattatgataaagcagatggttaagttggtaactaccagtgaatatgggtaaagttaggrtgttctttactctgttttgggggtgcaacttttctataagtgaaagtacttccaaataaaaagttaaaaggcaagcaaataaataaaagagacagtttctatgttatatatcctagctatgtttaccatgtctggattctgaaagctgcagagcagaaaacctgaagaacagatcacctgttcttaaaataccactgttggccagacatagtaactcactcctgtaatcccagcactttgggaagccgaggtgggaggatcacctgagctcaggagtttgagaacagcctgggaaacatagtgagaccctgtctctacaaaaatttaaaaaattatccaggcatcatggttcgtgcctgtagtcctagctactcaggaggctgaggtaggaggattgcttgagcctgggagttcgaggctgcagtgaaccatgatcacactaaagcactctagcctgggcaacagagcaagaccctgtatcaaaaaaacaatcaaacaaaaaatcactcctaattttcctcccttttagtacttttaaaaattaacttaaaacattttttggataattgtagttttttttacttttttttttttgagacagagtctcattctgtcacccaggctggagtgtactggtgcaatctcaggtgactgcaacctctgtctcctggattcaagtgattctcctgcctcagcctcctgagtagctgggttcataggcgtgcaccacacctggctcgtttttatatttttagtagagatggggtttcaccttgttggccaggctggtctcagattcctgacttcaagtgatctgcccgccttggcctcacgtgcagttttaggaaataatacagagatccccagcactctttccagttttccccaagggtaacatcttgcaaagtgagaggacgatatcacagtcaggatactgacattgataccatcaagatacataatgtttccatcaccaatcagtggtcatggtgccttttatagccaaacccacttctctcctaccttcccatcccttttttaattttgccagtcattaatctgttgcccatttctgtcattttatgaatgtcacataggccgggcgcggtggctcacgcctgtaatcccagcactttgggaggccgaggcaggcggatcacgaggtcaggagatcgagaccatcctggctaacatggtgaaaccccatctctactaaaaaatacaaaaaattagccaagcgtggtggcgggcgcctgtagtcccagctactcgggaggctgaggcaggagaatggtgtggacccgggagacggagcttgtagtgagctgaaatcacaccactgcactccagactgggtgacaaagcgagactccatcttaaaaaaaaaaaaaaaagaatgtcacataatgaatcatatggcatataaccgtttgagactcagggtaattctcatgagactcatccagcttgttggtgcatcaacagtttattcctttttattgctgagtaatttccatggtatggaggaaccatggtttaactattcacccattggaggacatctaggttgtttccagcttggagttattatgaataaagctgctgtgaacatttgtgtacaggtttcttggttttctggtttgttttaaacagttctagccaggcacggtggctcacacctgtaatcctaacacttggaaggctgaggtaggaggactgcttgatcctaggaggcagaggttgcaaggagccgaaattgtgccactgtactccagcctgggcaacatagcaagaccctgtcattcataggtaggtggatggatggatggacggacggacagatagataggtagaaatgtaaattacagggctacgctcagtggctcatgcctgtaatctcagcactttgggaggcgaaggcgggcggatcaccagaggtcagcagtttgagaccagcctggccaacatggcaaaaccccatctctactaaaaatacaaaaattagccaagcatgctggcatgtgcctgcaatcccagctactttggaggctgaggcaggagaatcacttgaacccaggaggcggaggttacaatgagccaagatcatgccactgcactccagcctgggccacagagtgagactccgtatcagtactttctttttattgtttttctgttattatagtttaagttcattgttattagattatatactctgtatggcttcaattcttttaaatttgttgaggtttgtttaatggtcaaagacatggtctgtctaggtgaatgttccatgggcttttagggaaaaaagtatattctagtgttgttgaatggtgtcttagtccattcaagctgctataacaaaataccgtaaactgggtgatttataaacaacagaaatttttctctcacagttctggaggctgggaagttcaagatcaaagtgccagcagattcagtgtcatgtgaggacgtgcttcctgcttcatagataagaggtacatacacgtttaggagcatcgtgtcttcctggtggatgaattctgttatcattaggtgatcctttgagcacttttaaaaagaatctgttggccgggcgcagtggctcacgcctgtaatcccaggactttggggggccaaggcgggcagatcacgaggttaggagattgagaccatcctggctaacacagtgaaaccctgtctctactacaaatacaaaaaaattagccgggcatggtggcaggcgcctgtagtcccagctactcaggaggctgaggcaggagaatggcgtgaacacaggaggcagagcttgcagtgagccaagatcacgccactgcactccagcctgggcaacaaagtgagaccctgtctcaaaaaataaaataaaataaaataaaaataatctgtttaatagcctactagtgttcttcctttactattttattgagcattaattaatcccaacattatgtctatgtcaggactgatgacaatatttggtataaaaatttgatagtctcagaggctgaggcaggagaatgcttgaatccaggaggcagaggttgcagtgagctgagaccgtgccactgcactccagcctgggcaacagaacaagactccatctcaaaaaaaaaaaaaaaaaaatcgatagtatcatatcctccaggattcaaagtgaacttcaaacagtcttatgtagtctaaattttggaatgcatcccagtattgagttgcagcagggatttgagtttttgtgaagagagagaggtatatcagaatcttgggcataaactaaggagccatgtcagaacctcaggtgtatgccaatgagatagatcagaacctcaggcatgtacccgatgagacagatcagaacctcaggcgtgtacccggtgagacaggtcagaacctcaagcgtgtacctgttgagacaggtcagaacctcaggcgtgtagccagtgagacaggtcagaacctcaggtgtgtacccagtgagacagatgagaacctcaggtgtgtaaccagtgagatatatcagaatcttgggtatttacccaaagaggtatagcagagtctcaggtatatactcaagaaggcatatcttgaggctttaagtatctagctaaggatttatatcaggatctcaggtttatacccagggaggtatagcagaatttggggtatagatctaaggaggtctatcagtctagagcatatagccaaggaactatatcagaacctcaggcacctacccaaagaggcattttaggactcgtaaggagggggtagatttcaaaagtgtagtctaacagtttatctactttgaaatttaaaacaatattaaaggaaaacatgaaatatttctatctgtcagaaggtgacatgagttttaaacaattaagaaatatactggctgtggccttgtaaccaaattattatgcctatagaaattacagactccattttccaggatagaataacagggactgacttaccttctcatctgagataacaaaacctccatacaaatacatgaaacaatgttcttcaagatgctggacatcaggcagtgaagggcactgatggttgtaagacaaggtgagaggtgtggcttgagagagtttccaggttgcagtgcagggagaggggaaactgaggcagatcttggcagacttcctcagttgacaaaatagagctgagagtccagggagaccatggtgtatagattatccaaagcaaagtatgagaggtgcaagccatatacagagggactccagagatctaccaaagtacttcttggtgcatccatatgagcaaaactacttgaggccaggaaaagaaccatctgagaggattagaaggaacagtgcccagtacttgtgccagccaggaatggtgcctgatactcacgcagggccaggaacagtgcagggatgtgagtgtttgttaggagagggaggtatatcagaatcttgggcataaagacaagaaaccatatcagaacatcaggtgtgtaccaatgagatagatcagaatctcgggtgtatacacagtgagatagatcagaatctcagatgtgtacacagtgaagcagatcagaatctcagatgtatacacagtgagatagattggaatctcaggtatgtacccagtgagtccaagagcatggtgctggcatccggtgagggccttcctgctggatcgtgacatgaagcaaggcaaagagcctgtcagctcagggctctcttcctcttcttataaagtcaccagtcctatcatgggggccccaccctgatgatcttataatcctaattacctcccaaaggctaccttcaaatgctatcaacatatgaatttggaaactaagtttccagcacatgaaatttgggggatacattcaaagtatagcaaatattacatcataaccagtaggattcatcccaggaaatgccaaatggcttgataatcaaaaattaatgtaactcatcgtattaacaggatgaaaaagaaaaaccatgtgatcatcttagtagatgcagaaaagcagttgattaaatcccacattcatttctaacttaaaaaaacaactggattttgacagaggtgcaaaggcaatttggtagagaaaggacagtcttttcaataaatggtgctggtgcaatggttatccatatgcccaaaatgaactttgacccatgcctcatgccatacacaaaaattaactcaaaatagatcagagatctgaaggtaaaatttaaaactataaaacttctagaagaaaacacaggagaaaaatctttgtgaccttggtctaggcaaagatttcatagatatgacaccgagaacacaatctatgaaagaaaaaaatcaataaattgaacttcatcaaaatgaaacttttactgttcaaaagacagttttaggagaatgaaaacacaagttacacattgggaagaaatattcgaaaagcatttgcctgataaaggtattgtagctggaagacagaaaaaattctcaaaactcacctagaagaaaataacccagttttaaaaatgggcaagagatctgaacaaacacattgtcaaagaagatagatgaatagcaagtaagcatgtgaaaaattctcaatgttatcagtcatcagagcaatgcagatgaaacctacagtccccatgctaatgttctacaacttacacagtggtggtatgataccactacatgcccatttgaatggccaaaattagaaaggttgaccataccaaacattagccatgatgtgcaggaactagaactctcatctttgctgacaggaaggtaaaatgatacaaacacattgaaaaacaggttggcagttgcttttttttttttttgagatggagtcttgctctcccaggctggagtacagtggcgcgatctcagctcactgcaacctctgcctcccaatgaattagaaaaataataataaaggtaacaatagcagtaataataatagaaataatgatagtttctttaataaaaatgctgtttaggcccagactgaaaggctttaagtaaccactcccccactgaagttagagttaagaaagaatattaattttccttgtgtgaaacattaatcttatctagcctccatgtattttgtaagttctgtaaattcctgttttccctgcacagctgcaagttcacaaggcagataagcttaagctgcaaaacatgtttttcttaagatgtaaggcatgtcacaagaatatcacaagatgataacggcctttattctcacttctgtatgcctgcttcctgcctcacatatttcctgcctcaagatgcgtaaaaggtacttgccttctttgtttggtgctctgactttctggatgcaagtccactgagccagtgtacaccttaaataaatcctcctgaaccccatcaatcgctccagttctctgatttcccactacattttctgggggctcgtccgggattggagatggcagattttctgtctcccttgcctgtggaactggagcccgggtcgagggagacctgggacctttggtgccaatgggaggactttagcccggaaaggagattggctctcctgcatcccggtgtccttcctagacagcacaacggaacctataaaggggttgcaggacggttccagcaggggctggggatggtgagagtagctcactgattcagatgacagggttttgccatgttgcccagacccagaggggctggggacagtgagagtagctcactgattcagatgaaacttacaccttagccgatgcaggacacgagagtggctcactaagttggtcaggaaagaaactgaaaatgggaagagtggcttcctgccttgactaaggatcgggaactgggagcggggaggtgtgtgaaagagatggttccgggagggccgtgatgtggggagacacagatctcttagcacggactgtgtgctctgaggcgagtgtgtgattgaccagaaccagggcatcacatacagctgacaggagctgccccacagctgcagcaggctgtggcaggaataaggtactctcctagctaagcagcacctgaaacttccgtaataggacccagtctggtcagtctggaacgaaagtgagagtgagtgtgcatcacaaagggcgggatgggaggaaaagcatcgaaacccactcctctggggtgcatgttaaagaattttaagaaaggttttgctggagattatggaattaagttgtcccccccaaagattgagggttctgtgtgaagtggaatggccttcttttaatgtcgggtggccagccgagggtacaataaatagggaaatgattggtcatatatttagggtagtgactggggttggaggacaccctgggcatccagatcagttcccatacatcaattcctggatgatcacagtctagacatgccccaaatggttacagccttgtctggcaacttactgtaagactctagtgacctgagccgaacctaaggcagttagagggcccccttcaccagacacctcaggtggaaagaaaaagccacaggaaaattaggaaagacctgttctacttcactgggatcaagtgattctcctgcctcagcctcctgagtagctgggagtacgggtgtgcaccaccacgcctggctaatttttttaaattttatttttagtagagacggggttttgccacattggccaggctggtcttgaactcctgacctcagacagtctgcctgccttggcctcccaaagtgctgggattacaggtgtgaaccaccatgcccagccagcagtttcttataaagttaaaccaatgcctaccatgagatctggcaatcccactcctaagtatttggccaagaaaaaagaaagcatatattccatacagagtctagtcctgaatgtctatagctgctttatttataatagctcagacttggaaaccattcagatgccattaataggtgaatatattctcaaactgtggttatccatacaatggagtattactttgcaatcaaaaggaatggcctatgaatacccataacaacatggatgaatgctgaaataattgtgctgagtaaaagaagacaggaaaaataagtataatacatactgcttgattctatttgtataaaactagaaagtacaaactaatctgtaatgacaggaagcagaccagtgacagtgggcatggaggggcaagagggagagattagatgggcacaggagagctttgaggatgatgggtctgcgtactgtctcggctatgatagtggtttcacaggttgatacatacggcaaaaaataccaaatttgtacactttaaatatgtacagattattgtatgccagttacatgtccataaagctttcttttgttgttttgtttttattttattttttgagacagagtctcgctccatcgtccaggctggagtgcaatggcaccgtctcagagcactgtaacctccgcctcccgggttcaagcgattctcatgcctcagcctcccaagtagctgagactacaggcatacgccaccatgcccagctaatttttctatttttagtaaagacagggtttcgccatgattgccaggctggtcttgaactcctgacctcaggtgatccacccacctcggcctcccaaagtgttgggattataggcatgagccacagcaccgggcccataaagctgtcttttaaatgaaaaaaagttgtcttgaaataagcattagaactgtggctttggctctgaaatcctcatctgaggacccacactcgggtgccccaatgtggcggtgcttacagaaatgactccatctgctaaatgagtaaatgggtaattctccactgaacacacactcgtttagcagcataagcagcaagagttcaggtaatcctcacattgcaatttgtcattagtttaaacttccagtctttgttttaaaaacacattagaataatactacattttccctcatctctaaacttgactgaagactccaagagagagtaatattcatcaagaggatcatctactcaacacagataaactgggaaagaaaaataacttgtgagtaattcagaatctggattatcaggtcaggctcaatggctcacgccagtagtcccagcactttgcggggcccaggagggcagatcacttgagttcaggagtttgagaccagcctgggcaacatggcgaaaccctgtctatacaaaaaatagaaaaattagccaggcatggtggcatgtgcctgtagtcccagttacccgggaggctgaggtgggaggatcacttgagcctgggaggtcgatattgcagtgagctgtaattgcaccatgcactccagcctgggtgaaagaaggaaactctgtgtccaaaacaaaacaaaacaaaacaaaaaaagctaaattatcaaatgtctagatcgttgatggttggaagtaaagttgagaaatgttcacactgggagatgacacacagtaaaccacacagagggttctaacgtggttgttagaagcagaaactagaggcttgctgcctgaggtcaaaccccggtcccggtggttctgtgccccagcgcatgttgtggtagcctctctgtacttcagtttcctcatctgtaaagtaaacataatgataatgcctgcctcatggggttgctgttaccaggaagtgagttaatgcacattaggttcttatttatgacagtgcctggcataggataagggctcaaaaagtgttagctggaactactatcattatcaacatctctaatttattgcagggttggatctgaaaaatggctgatgatgatttgatgatgacttcatttttataaaacaataatattgcagtgcaaattaaacacaagcaacctgcaacacgccactgcaagttggatgtctagaaaaggtgccatgagttaccttctaaaacatcataagaaaccatgttcaccaataattaccataataggagagaagtaccaagtaccatggggagacagagtccagaatctcagagagagacacagttaccttttagttacactaatggggaaaacaggagctttgctacccttgcacctgatggagggcttctctgaatatagggctatggggtcagaagccagtttcctcccatatttagaaggtttccaactgttatctttcacttcccatgttgctgttggaaaatccaaaagtattctatttggccaggcacagtggctcacacctgtaatcccagcaatttgggaggccgagattacctgaggtcggaagttcaagaccagcctggccaatatggcaaaacctcttctctacaaaaaatacaaaaattagccaggcgtggtggcgcacgcctgtagtaccagctattcgggaggctgaggcacgagaattgctttaacctgggaggtggaggttgcagtgagctgagattgtgtcactgcactccagcctacgcgacagagcaagactccgtctcaaaaaacaaaaaaagtattctgttggatcgtttgtgtgcgacgtgtttttccctctcagaaagctcgtagggtcttctctttgtctccagcatggtctggaatttcccagtgagtgaccatctgtgtgtgtgtattcatccattccatggagtccctgctgggcccttgcaatctggaaattcatgcccatcatttctgagaagttatcctgaacgtactggttggtggtttcctgtgctccatgttcttgcttcctgctcttcggagctcctgttatttggttgagttttgtctcctggactggtcctctcttacttctcttgcttctcccatgttccatctgttttcactctactttctgtgagatcaggccctttatcttccaacccttctattaggtttgcaattgagtttgtaattccctagaaaagttcttattctctgaatatcccttttgatagcatactcttcctttttcatgagtgcagtgtttctgcatggctctcatcagaacatgctagtgtctcccatttctcataactttctagagtgaggaccatttgaacttggagtgccctcattttaaaactctgtggttgaccttgttcccctcttttgctgctgcttttcaccagactttgaagaagcagaagtacattcagaacttgtctgctctggcaaaagacaatactgttggcttaatctaaaaattgaagaagaaagctcaaggagagaagtttaaaaatataccacctctggctgggcgcggtggctcacatctgtaattccagcactttgggaggctgaggcaggtggatcacctgaggtcaggagttcaagaccagcctggccaacatggtgaaaccccgtctctactaaaaatacaaaaattagccaggtatggtggcagccgcctgtaattccagctactcgggaggctgaggcaggagaatcacttgaacccaggaggcagaggttgtggtgagccaagatcacgccactacactacagcctgggtgacaaagtgagactccgtctccaaaaaaaaaaaaaaaaaaaaaatatatatatatatatatatatatatatatatatatatatatatatatacacacacactaaccttcagcatataggactattgcagaagggattatctttctacttggttggttcctcttggtcttgagcaaattttgacttccctgagttggcccctaaaagttaaagggaaagggccttttctctttcctttaaaaccaatatggcatatttgctgagaacttagataccacaggattggcagtgtagacttacattcatagaccggatgccatcagccaaccttgagtaatttgcagcacactgcatcattttatttaagtaatgcgaagtccttgacatgtctcagacattgtcttggttacttgtaaggtctcacataaatctaattttcctctttctctgccctttctggttcagctcagtttattcaagggtgtatttgtgcaacacacttgaataaggtgtggtcccgcctttgtagatgttatagtttgggaagaccagccgggcagagagaagagcatgattcaaggatgaaggcgtgggctgggggccgagggagcaaggattcccagtaacgagggaggaaggagcagcaccatgtgcccattactctatagacatctcgaaccacctggccatgtagctgtcattaacctaaatttacagttattgaaactgaggttcagccaggcgtagtggctcacgcctgaacacaggaggcggaggttgcagtgagccgagatcacgccactgcactccaggctaggtgagagagcgagactctgtctcaaaaaaaataaataaataaaagaaaagaacaaaactgaggttcaaagaaatgtacagtgctcccccccttatccaaagaggatacactccaagcccccacagtggatacctgaagcctcagatagtatcaagccctatatatgctatgttttttccctgtatatgcatacctatgataaagtttataaattaggcacagtaggagactaacaacaatgataataaaatggaacaattataacatacactgtaacaaaagggtctcttcctctctctctcagaatatcttattgtactgtactgggggtaactaaaaccaaggaaagtgaaaccatggataagggatatctactgtataaggtggagttttcaaggtcatagcactgccttcccctgaggttggccttgcagcctctctaggcactgctgctgctgctaagaacccctgtgaggtgaacactgtaagcatcatcattgcttctcagaagaggagacctggcttacagaggtcaagcctcaggtaccttaaacaccattttaaaactgaactcatggccaggtgcattggctcatgcctgtaatcccttctctccatgctcaaaacctgccctccttgtctttatattccaaatttcgtgggtgccacctcctctgcccagtgacttaagccagagcatacattcatcctagactctgtcccaggtccctggtccaggcagctgccagttgtcaggatcagctctttatctcgcagtcctcctgcctcttgtgtcattacccagggctgtcaccatcttttcttgggacagttacaacagccccgtaaggagttgtgctgcttctagtcttgttccctttgaatctggattccttcttgccatcaagacaatcctgatacaaatctgatcacgtcacacttcccttcaatagtcttccatggctccttattgttttaggatgaaatccaaactcctaaacatggggattaacaatgtgccatgattggcactgctggcctctcctacctctgcagactcacctcttgccacttctcccttgaggtagatcaaaaatggtcacaagttctttgaggctcttcccatcaagaggtagagtttatttccccacctcttggatctggcttgccttgtgacttgctttgacccacagaacgtaacagaaaggacactgcctaacttacaaatgaggtctaccttaagaggctttgcagattccacattcaacctcttggaatgctgccaccatctgagaagcctgaggtggcctctgtgaggatgaaagacttcatggtgagaaatacctagcgaacagcctggcaccagctaccaggcatgtgactgaggccatccagccatagctgagccacaaaatgaccacagctatgtgaattatcccaggtcagaccagtagaagagccacctggctgagctcagcccaatttgctgacccatagaattgtgaacaaataaaatggttgtagttataagccattaagtttcagagtttgttacacggtaacatgtaactgatacaactcttggagccagttgttcagccattctcaaccacttattcaatgatgttttgggccatatatgcagatatgctgttcccttttccttgaaatggcccttaccctcctttctgttgggttttcctatggaatatccagtcagcctttaggattcatcttgggtgtcccttcctgtatgtaggctccctggccctccaggattcccccagtaacagccctcatcatgctgcctttgcaaccatttgtttatttgtacctctcacctgctagttgggcaagttactcacttctctcaacctctgcatttttcttctttataaatgggaccaataatacctaccctgccctggcgtggatagattaaagaaaaaaaatacatgcagctgccattgagggcctggcccacgtgtgatgttcaataatattatttctccttgttttccttcccgtgccagtgccacacccccctgtcccagtgcactggggctgtggatcccttcaaagctgagattgcctgtctgtggtctccagcgttaagcacagtcattagctcaggtgcgtactcatgtgttccacgagttcaagcctcagccctgtaaagtttgcctgccgtgtatctgatatatttctgctaaaacccattaggcctttcttgctctgaaatgtcatcgttagttgtgtgtcacttcagttttgtaactggccaggccactgcgcccaggctgcttcctcgtcatctggctgctaaatgcttcaaccttacctgccttgctatgcgtcccatcctgtatcaggtcagagctcttgagtggtgaatacaaatttcatttcagttgacttttgattcttgtggcaggcctctcggcctactctaatttgattgcaacggacacaaaatgtgtccaaacttgcagcttttcttctcttattttgatatcaccatccacaaaggtaagatattttaaagcaataactacaaactttctgaaaattatgaagaagtgctgggttttaaatggaagtcatatagtgtgaactttgtgtaaagtccgtagggagttttcttggaaatggctgggaacattctttttgcacctttgaagataaaggtaggtggaggagctcacagctcttgtgccatgttgggcttgtcactcttgtttatgtgccaaattcttttgattacaaaattttaagtttaatgctttaggtattgttgggcaagatctagatgtatctagttaaatgtaggtgatatgcaaactatttatgatgtatttgatttaaattcattaagatagagtgtctttaccaccattatagtctggtccttttccttctgttttaaatgtgtttccattggcattttctaaactgactttgttagcgtgttaatcatttggcactggtaatgattaatcttttctttctttctattttttttcttttttttttttgagacagagtcttgctctgtcaccaggctggagtgcagtggcgcagtctcagctcactgcaacctccgcctcccaggttcaagtgattctcctgcctcagcctcccaagtagctagggactacaggcacgtgccaccacgcccagctaatttttgtatttttaatagagatggggtttcaccatgttggccaggatggtctcagcctcttgacctcgtgatccgcccacctcggcctcccaaagtgctgggattacaggcatgagcgactgcgcccagccgtgttcatctatttctgtgaaccgatgctaggtgaaggtacagagggctttctagcttctgggtttgtttattctgaaatgttattttaaatcttagcccaacaaattgagcgaaaagacttctagatgttaaatatgatattcaaaaaatataaagacaaggtgataaattagaattggtgggaaagagaaaaatctgtcttctgatggtcacctgccccagcaacactactcgtttgagaagacttccatcctttaccctcaaagtgttccatgaggttggatcagacatcatttagcaaagaaagatgtaaatagatttctgtagggtggcattattaagcatattaagtggttacaatacagtaaattagagggagtagtacagaagcataagcagtcaaaaaagtgaaagtctaacgttcgtaattattgttctggaggcttttgtatcacatataagttccaggctgggtatgatggctcacaccagtaatcccaacacttagaggccaagccgcgtggatcgcttgagcccaggagttcgagaccaggctgggcaacatagtgaaacctatctctacaaaaatacaaaaattagctgggggtggtggcagcgcctgtagtccaaaccacttgggagcctgaggtgagaggatcacctgggcccgggagatcaaggctgcggtgagccatgatcttgccattgcactccagcctgagtgacagagagagactctgtctcaaaaataaaaaagttttgagtgtgaaaattcaagctcaattccatttgttggttgtcttgagtgtctgatcacatagaatataaagatgttttgatagttgggacagtattcagctacctgctatttaatacattatttcagaaaatatttacaaagggggctgggcacagtggctcatgcctgtaatcccagcactttgggaggccgaggtgggcggattacctgaggtcaggtgttcaaaaccagcctggccaaacatggtgaaaccacatctctactaaatatacaaaaaattagccgggcgtggtggtgtgtgcctgtagtcccagctactcaggaggctgaggcacgagaatcgcttgaacccgggaggcgtggggttgcagtgagccgagattgcacaactgcactccagcctgggtgacagagtgagactgcatctataaaaacaacaacaaaaaagaaaatatttgcaaaggaccttctgggtccaagaacctcatgtccaatacaagggtgcacacgtgggtgagacacggcagctgctctccagaagcccacagtggaggggtttcccttcggtctccttttattccaagcaagtggcaaaactactttactcttaatacaaaccacttccttttatcacaggacgcttcccaagctctgcaactgttgctcctgaggaagggagtggaactgataatctgttcctccctattgtgttcagtatggttttttttttttttttccttttgctggctttgttttcctgtccctgtgatgattaaaattcactctgcaaattagatcacctttcccacgcagagtccctttgacttctgttctagatatccattacatttttgtagtcttcggacacactgtgtgtgccgctttgccctctgggtgacagcaggctgtggctgcggcgacagagctgaggtgaattctcacagaccatcactgggttactcctggagtaagtaattcccaagagctccttctgtgcagatcgttagaaatagatattgaggccaggcgcggtggctcatgcctgtaatcccagcactttgggaggctgaggcgggcagatcacgaggtcaagagatcaagaccatcctggccaacatggtgaaaccttgtctctactaaaaatacaaaagtagccgggcgtggtggcgcacgcccgtagtcccagctactcaggaggctgaggcaggagaatcacttgaacccgtgaaacggaagttgcggtgagccgagatcacgccactgtactccagcctggtgacagagtgagactccatctcaaaaaaaagagaaagaaagaaatagacattgaacacctgctacacagcagggattgtgctagaagtatggatgcaaagattaggtgaatatgttccctagcctcacaaagcatacagtctagtaggagagacagacacgtaaaaagtttcaacagcacagataatcagggctacaccagaattgggcccaagatgctgcaggaatctataggtgaatgggtttcatgaaggaagagcttcttctcccatttataactcattttagcctaatcttccaaacagtcacgcatctaagagcaggtgatgcagaaaataccctcgtgttagttatgaattaccgttggaatccttccagtgtttgcacctgccctgtgctcgggtaacataaaacagtgatataatttgatgtctacttcctcttgtatttgtctgtttttaagtgttctacaattttcatatactctgtttcatcgttctcaaaggaatattttgattgataaatgtttagttagtaagacctaaaaactgaatctcagtagtttgagcttatgatatacaagatgcagctctaacatttaaattggaagggaaatgtcaaaaagatacctgcactcttgtttgttgcaacactgtttacaatagctaagatttggaagcaacctaagtgtccatcaccagacaaatagataaaggaaatgtgatatatatacacaatggagtactattcagccataaaaaagaatgagatcctgtcatttacaacaacatgggtggaactggagatcattatgttaagtgaaataatccaggcacagaaagacaaacttcacatgttctcacttatttgtgggctctaaaaatcaaatcacttgaactcagagagattagaaggatggttaccagaggctgggaagggtggtgaggggttgggggcagtgaagatggttaacaggtacaaaaaactagaaagaatgaataagacccactaggttttttgttgttttgttgttgttgttgttttgagacagagtctcactctgtcacccaggctggagtgcagtggcatgatctcggctcactgcaacctccacctcctgggttcaagcgatcctcctgcctcagcctcccaaatagctgggattatgggcacgcgccaccacacctggctaatgtttgtatttttagtagggaccgggtttcaccaggttgaacaggctggtctcaaactcctgacctcaagtgatccactcgccttggcctcccaaatgctcagattacaggcgtgagccaccgcacctggccactgtttgatagcataatagggcgactatagtcaataataacttaatggtatatttttaaataacttaaagagtataattggattgttgtaactgaaaggatcaatgcttgagggcacagctaccccattccccatgacgtgtttagttcacattacatgcctatgtcaaagcatctcatgtaccccataaatatatacactgagtatataccacaaatattttaaataatttttaaaataaaaaaataaattgtaagggaaagaaaattatgaatttagaaatgtaaaaggtctcaggtaaggaaggaatgagaggatcatgcagaacctcccatcattgctgggactggaacagaagccctaccttttcccaacaccctatccacctgtccctcacctctcagcttttgtgagactctgtctgtgctatgaaactgaagatctaattcagtgctgtttgcattgtcttgcctcctggaccagaggttgcagttgttgagaaaagggatggttggttatgccttgatcccccccagagcatttggggcataggacacgggaactggccagcctggttcactcttctcgattagctggacagcggcatgtcatgtgggtaataggaaggggtggggacttccccgggatattctgctcctgatcagaagcgccagtgatgtggggggagccccagcaccagagcatgctgggagggcgtgcagggtggggcaggtgcccgtttggcctctgctgtctatctgggggatgcatccaaaggcaactgttccttatctgctcttgttgggagcaaggaagggccaatttgttcaatgatccgtatacagccagtccctctggccagagttcaagacagtattgcctcactctatatagagattgtatcttggttagctcttcattcatagcaagaccaatgtttctgtaaattaatcctggtattgtttaaaagcaactaaaaatgatgaaattgtaaaactttgaaactccctgaatataacgacaagcaaactaacattgttttattggtcgatgctcctggccagaagagagaatattagcagggataaaaggcataggccacatgcattttccaccccagtgctgagaacacgatgggcgaaaaagggaggtggccacagcccatccatcacacagtctctgcccatctacttgctttttcctttttttttttttttttttttgtgacagagtctcgctttgtcacccagactggagggcagtggtgcaatctcagctcattgcaacttctacctcccaggttcaagcgattctcctgcctcagcctcccgagtagctgggattacaggcacctgccaccaagcccagctaatttgtttgtatttttagtagagacggggtttcaccatgttggccaggctggttttgaactcctgaccttaagtgatcagcccacctcagcctcccaaagtgctgggattacaggtgcgagccaccacgcctggccccagctacctgttttctttcttttttttttttttttttctttttttgagacaaagtcttgctcttgtcccccaggctggagtgcaattgcatgatctcagctcactgcaacctccacctcctgggttcaagcgattctcttgcctcagtctcctgagtagctgggattacaggcgcctaccaccacgcccggctaatttttgtatttttagtagagacggggtttcaccctgttggccaggctggtttcgaactcctgaccttaagtgatctgcccgcctcagcctcccaaagtgctgggattacaggtgtgagccaccatgcccggccccagctacttgctttctattgggatgaacctcatggttaatacagttagttagtgactgcaacttttgaactttttgttcatagtgaaaaatattttaagtaatgcttaccccattatgtttcttgtcatttgaaaaaaaatctcccttcagacagaatgcagaataaaatactacagaaaatctgtacagagtcccagcctgacttatgctagtaggttacagagaaagaaagtcttctaaaccctatgaaaggttaacagttctcttatttttccctgtgtgctatttgatgatttccctgtgaactttgatgatttattgccagaattccaaacataatatgtgaatttcacaaaaatggatgaaatgtatctatttttcattggtagaagaagccaaaacatcccttcctcaccgcactaaaagctgttgtttacatgaagcaaacctcaaatgtgaacatatttttacgcaaatgcatttaatgggtgaatatttgctttgggacggtattctttactctatctggagagtctggcgttccgtaatcaccatgtgatgacggctgccctgacagtggctggtagcagcacatacccccgagcctctccgtggtgtgcgccgtgggcaccatgtgaccattttcagaaaggaagacagttctggaagctaaaggtcacctagtcagcctcgttgggtgattgatgactcagctgggttcagggaggtggacccgaggcagagcctctagaaggcagcggtgggcagggcggttcaggcaggtggcacctgggcaaaggtgcagacgtggaatcctgaaagcaattctcagcgctgctgcgtttccaggaggtagaagaacagtgacaagtgcacagtcgggtagggacaaatgtggaagggctgggaacagtgtgttcaggagactgggcttcaatctggaggtctcaggaagtggtttaggatgtttcagcgagagcatgatacagactaacccaggaagaaccgctgctttgtcacttatacccctatggaaatgccgttcgctttgctagttgaaatagcctaccattgtctgggactcacccagttagatttgtttggactccacaaagtattcttgaccatacaatcatggtcgaggaccccctacatgagctgccttcatggctacagggagagcacaccaaagtggatgtcacacccagcacacatgccaccggcttggccctgcgccccgcagcctgagccacactggctgcctgttcctggaatgtgccaacatgtttcagtcctggagcctttgcacttggtgttctcttcgctggaacattctcccccaagacatttacacagcttgccccctcattccctgaggttatctcctgccccctaatcagtgaggccttccctggcctcaccccggacactccacacgtgcattcatttcgttgttcaccatctgtgtcccagttacaagggaggctccctgagagcagggatctgatttttgttagttgttgttgttgctgttttgaggtggagtcttgctttgtcgcccaggctggtgtgcagtggtgcgacctcagctcaccgcaacctccgcctcccatgttcaagcggttctcctgcctcaacctcctgagtagctgggattacaggtgcctgccaccatgcccagctaatttttgtatttttagtagagacagagtttcatcatgttggtcaagctgccctccaactcctgacctcgtgatctgcccacctgggcctcccaaagtgctgggattacaggcatgagccactgcacccgaccctgttttttgtttggttttggttttggtttggttttggtttttttttgagacacggtctcactctgtcgccccggctagagtgtggtggcaccatctcggctcactgcaacctccacctcccaggttcaagtgattctcctgcctcagcctcctgagtagctgggattacaggcacatgccaccacacccagctaatttttgtatttttagtagagatggggttttgccatgttggccagtctggtctcaaactcctgacctcaagtgatccgcccgcctcggcctcccaaagtgctgggattacaggtgtgagccactgtgcccggcccagggatctgtttttgtctccgctgtgtccccagcacctcaaacatattgtacggagctgcgacagctgcgcagtcagtgatgactgagagattcctggccccgtgggctatggctccttcaacagtttgttgtttaaaggttcttcactttctcagcgtgctgatcaagagacaagcctggaggagaggctcagtggtgctcctgtgtagatgatgaattcaggtgtatcttggatggtaaatgacgttgcatttaaaaccaagcaagtggccaggcgcagtggctcacacctgtgatcaaagcactttagaaggccgaggcgggcggatcacctgaagtcaggagtttgagaccagcctggccagcatggtaaaaacccgtctctactaataatacaaaaaaactagctgggcgtggtggcgggcacctgtaatcccaaccactcagaaggctgaggcaggagaattgcttgaacccgggaggtggaggttgcagtgagctgagatcgcaccactgtactccagcctgggcgacaagagcaagactctatctcaaaaataaaaaaaattaaaaattaaaatttaaaattaaaacaaacagccggacgcagtggctcactcctgtaatcgcagcactttgcgaggctgaggcgagcggaatacgagctcaggagatcgagaccaccctggctaacacagtgaaacccgtctctactaaaaaaaaaaaaatacaaaaaattagccaggcgtggtggcaggcgcctgtagtcccagctactcaggagactgaggcaggagaatggtgtgaacccgggaggcggagcttgcagtgagccgagattgtgcccctgcactccagcctgggcaacagactgagactctgtctcaaaaaaaaaaaaaaaagaataaataaataaataataaaaaataaaaacaaacaagtgaacgttgttatacgtcagtcttaccaattgttcctctttcctcccagtagcttggagctcggcggcacaaccagcaccatctggtcgcgatggtggacacggaaagcccactctgccccctctccccactcgaggccggcgatctagagagcccgttatctgaagagttcctgcaagaaatgggaaacatccaagagatttcgcaatccatcggcgaggatagttctggaagctttggctttacggaataccagtatttaggaagctgtcctggctcagatggctcggtcatcacgggtaagtgtgccgtttcctagaaagttttatttagaaatgtttcttcctccaagaaaactgttctctcttttttttttttttttttttgagacggagtctcgctctgtcgcccaggctggagtgcaatggctcgatctcggctcactgcaggctccacctcctgggttcacaccattctcctgcctcagcctcccgagtagctgggactacaggtgcccgccaccacgcccagctaatttttgtatttttaatagagacggggtttcactgtgttagccaggatggtctccatctcctgacctcatgatctgcccgcctcggcctcctaaagtgctgggattataggcgtgagccaccgcgcccggccgaaaactgttctctttagctggaaaagaagtcacacttttttgcaaagaaagcttcagacgtggtaaagcatgacctccagtgcccctgggccctggaaggcgcgtgtcacggctcacggtgccccctcttgtgaaagccatgcacacatcaaacagtgcttgagattcagtcacggggaacagctaaagtacacagaccctaaccccagcaagcccgcggggggcagctagacatttttaagaggagacgtgtgcaagggtctgcatagaggtactgttggtaagagggaaggatgggaaacaagctgtacatgcgtcaaagggaaacagataaattgggatgcatttatacagtggtatatacttcatagcaatttaaaagaacagactaggctaggcgcggtggctcacgcctataatcccagcactttggaaggccgaggcaagtggatcacttgaggtcaggagcttgagaccagcctgaccaacatggtgaggccccatctatacaaaaaaaatttaaaattaaaaaaaattagccaggcatggtggtgcatgcatgtggtcctagctactcaggatgctgagggaggaggaccacttgagcccaggagctcgaggctgccatgagctatgactgccactgcactccagcctgggtgacagtgagaccctgtctttaaaaaaaaatttttttaagcaacattgaatgaaaataaacaagcttaatgaatatttttatgatccaattaatgtaaaatcttttattttttattttttgagacagagttttgctcttgttgcccaggctggagtgcagtggtatgatctcagcccactacaacgtccatcttccgagctcaagcagttctcctgcctcagcctccctagtagctaggattacaggcacccgtcaccatgccgggctaatttttgtatttttagtagagatggggtttcaccgtgttggccaggctggtctcaaactcctgacgtcaggtggtccgcctgcctcagcctcccaaagtgcagggatcacaggcatgagccactgcacccggcccaattaaaatctttaacactaaacaatctagtacatcactggtggaaacagacatacacctattgcaaagggcatctcagctttaaggactcagtcacctcctgagcaagatggagggagaactggggaggggtcccatggggactgtaattctctctaggttgtatatttttaaaagacttcagcagtgtgataaacctgggtggtgtgtacatgggtattacagtcatgttgcttaatgacagggacaggttgtgagaaatgcatccttaggtgatttcatcattgtgtgaaagtcatagagtacacttaaacccagatggtagagcctgctgcacaccgaggctctgcggtgcagcctgttgctccaaggcacgcacctgtacagcgtgttactgtactgaacggcgtaggcccctgtgacacaatggtaagtatttgtgcgtctaaacataccaaaacatatagtagaaaaggttacagcaaaaatacagtattatcatcttatgggaccatgataccacagttgaacttatggtctattgttgaccaaaatgtcactgtgcagtgtgtgactatacagaaataagctcagagaaattaagtaacttggctgggcgcagtggctcacgcctgtaatcccaacactttgggaggctgaggcaggcggatcacccgaggtcaggagttcaagaccagtctggccaacatggcaaaaccccatctctactaaagaatacaaaacattagctgggagtggtggcaggtgcctgtaatcccagctactctactcaggaggctgaggcagggagaattgcttgaacccaggaggcagaggttgcagtgagcagagatcatgccactgtactctagcttgggcgacagggtgagactccatctcaaaaaaaagttggggcgtggtggctcatgcctgtaatcccagcactttgggaggctgaggcgggcggatcacttgaggtcaggagttaaagaccagcctggtcaacatggtgaaaccccatctctactgaaaatacaaaaattagccaagcatggtggtacacacctgtaatgcctgggcaacagagcaagattccgtctcaaaaaaaaaaaaaaaaaaagtaagtaacctgccacggttcatacagccagaaagacacagagccgggcctggaccccgcctctcagcttgctctagagggctattctctgcatgctggcatgatcgcgccttgtaaaaggtggcagtgttctcagcttagtcaatcaggaattgcaagaggcaagtgagcccctgaggactctggggggcctttgtgaccgagcagctttgggagtgaccctgacagacctttacaggtggtgcaagttttgactccctttctcctggcgcgttaagcagaggataagcgctgtggaaggagtgaaggtgtagggagatcatggcccccagagcagtggggaaggggacagggaggctggaggagagcaaggaaaaggctccgtgtcaggtggcgccttgagtggcctgggtaggttgtcttgcagtgaacccgggttaatggccttgacaatgaccgcattgtttcctgagcactgcaggctgcccacacacctcacacctcggcttgcctaagcccagagcagccttgtgaggtcgttgttatgtttatttaaggaaggaggaaaggaggcaggtcccaggacatcctgacgtgctggagatcaccagcccagaacccagctcttaaccccacaatgtgggacctttcttcacccatcacagacacaccccatgctggttcaccgttttcctataatgactatttgtgctatttattagaaaaatcttttccttatggatttgaaaagatttatcttgcttttgtttttcttttttgcctttcttttttaaggcaggcaggctcccgcagccccacccccagggtgaaaaatatagttcattgtctagtaaaagagttcagagatacacttttttctttgggtaagatatactctagagcttgttctgaaatatggaatttgtgtgagctgcgggagtgggtgggtgtgtggctctagctctggaaagttctttcctggcagtggccaggagggctgcccagccccctcctgcctcctctggcagcttaaacacaggaccccttattctgtgctctctcctgacccctggtcctcatgcaggagggaaccctgctcttctagggtccttttctaaaagtagtgtcttttaggtcattgtcaagaactataatctaaaatgtatttttaactcatctggaaattctgacagaggtaaggcttgagaatttcctgcatactagccttgtggtctatataatccattaaaagccacatttaacccaattccacagactgaactgtgcttcccatctaaataaattaaaagcaggccgggcacggtggtcacgtgtgtaatcccagcactttgggaggtcgaggcgggtggatcacctgaggtcaggagttcgagaccaacctggccaacatggtggaacctcatctctactaaaaataaaaaaaattagctgggcgtggaggcgtgcacctcttaagcttaaggacatatttcttatgatccaattaatgtaaaatattttattttttatttattttttgagacagagtttcactcttgctgcccaggctggagtgcagtggtgcgatcttggcctgtaatcccggctactcaggaggctgaggcaggacagtcgcttgaatccaggaggcggaggttgcagtgagccaagatcacaccactgcactccagcctgggcaacagagtgagactctgtctttaaataaataaataaataaatagcgagggttcagggcaggagaaaaagggttccaaatttgttctgaaccaattccaaggaactttatggcacaaagaaaaaaaaggggaacttacaaaaagtgaccacactgaagcgtcctggtcacccatccctggttttgaccaccagcctttaaagtggcaagcgggtgataacccatttcttatttccccctcagcatttcctcactgttattcatacatgtggtcatttgtactcatctcacaattgttaaaacctctttcctcccttccaggttttactgaactgttactgcgaagtctgagagatgaggtcatttaagattatttcttatttgtaaattagatcgttcatatttgtacctaatctgatcttttgggtaatattcctagttatgtagactggtctctcagaagagccggatattaaatgcagtactttaaactttacacccaggagaccggatgggtgaggctggttcactcggccaaagtaccattttatctctgctttttcttcccggctttattgccataattgacatacaataaactgcatgtatttaaagtgtacaatctgttgggtgtacacacacacgcatctgtgaaaccatcatcacactcaaaatagtgatgtagaaattttgctccttagttcgactaaatctgggttcttgtgtcatgaccaggaaaaattaggcacgtggacacgttgaagggtgaggagagcagtattgggcgaaaaggaaaaaagaaaaaaactctcagcaaagctagaggggatcctgccaatgagttcccagctcacagactgattagcaggccaccacacatgagctggaggccaggctcctcccgctgcgcaaggtgagaacttcccgtggctccaccccattctcccaatgcccaggtgggtccccgtcccttgcgggcctgtccagacaagggaaccctgggcaggttccctcatctacacaaaagcacctgaggtaaacacttgtggggcaggttgcagattctctggggacgccccccttctctgcctcctgcatctatcagtagtgcctctgtctgtcacccctaaagtttacttgtgctgtttctaattcctctttccccagccccgtgcctccctgcctccctcccccagtaaaccatgaatccactttctatcattctaggttgctttatatttcctagaattttatataaatggaatcatacagcacgtactctttctaggctggcttctttcactctgcagaatggctgtgagactcatctgcattgcagcaagcatcaatagttcattcttcatccatcatgtggacatagcacagtttgctgattcacgcacctgttgatgagcatttaggttgtttctagcttatggctattacaaataaagctgctatgaacattcacgtacaagtctctgtacaaccctctgctttcatttcttttgaataaatacctaggagtatgacggctggaacagatggcaggtgtttgtgtaactttttaagaaactgccaaaatcttttccagcatttcagaaaaatcttagaaaatgctatactatgttatattcccactggcagtatatgggggagttccagttcctccataccctcatcaacatgaggcatgatcagtctttttaattttaaccatgtcagtaggtgtgtgatggtctctcactgtggtgatttttatttgcacttccctggtgattttgagcatcttttcgtatgcttatttgccatatatcttctttggtgatatttctgttcaaagcctttgctcattttttaattgagttgcttttctactattcactattgaacactatttatatattttgaatacaaatactttatcagacatgtgatctacaaatattttccccagtgtgtggtttgtctttcttttctttctactgatagtatcttaaaaaaaaaaaagaaaaaagattgttttgtttgttttgttttgtttttgagatagggtctcaatctattgcccaggctagagtgcagtggtgcgatcatggcttactgcagccttgacctcttgggctcaggaaaccctccgacctcagcctcccaagtagctgggaccacaggtgtgtaccaccatgcttggctaattttttttttttagatacagagactcgttatgttgccaggggtggtcttgaactcctggactcaagcgaccctcccacttcggcctcccaaagtgctgggattacaggtgtgagccatcatgcccgaccagttcttaattttgatgaagtccaatttatcaatgtcctttttttatggatacttcatttatttatttatttgagagagggtctcaccctgagcccaggctggagttcagtggcatgatctcagctcactgcagcctcaacctcccaggcccaggtaatcctcctacttcagcctcccaagtagctgagactacaggtacctgccaccatgcccgggtaagttttttgtatttatttgtagagacggggtttcgccatgttgcccaggttggtctcaaactcctgggctcaagtgatctgcccatctcagcctcccaaagtgttgggattacaggcgtgagccaccatgcccagccatatatatatatatatatatatatatatatatatattttttttttttttttttttttttttttttgagacagagtctcactctgttgcccaggctggagtgcagtggtgcaatcttagctcactgcaacctccttctctgaggttcaagtaattctcatgcctcagcctctttagtagctgggattacaggcatgtgctaccaggcccggctaattaccagccttatatttttgaactctgtttaaaacatttaggtgcataaacattcaggcttgttatattctgttgatgaactgaaccttttattattatgaaattgctgttgtaatccgtggtaaaattatttgttctgaacactactttgtctgttattgatgtagccactgcagctttcttttgattggtgttaacatggtatatcttttcccattctttttcttttaactggtttgtgtctttatactatggtttgatttaaatctattatctcacaatttgttctctttggtacatctttgttttgttcccttttcctctttttatgccttctgttgaattaattgagtcttttttgttttgtttcatttaattttgttttttgagacggagtctctctctgtctccaggctggagtgcaatggcgctatctcggctcactgcaacctctgcctcctgggttcaagcaattctcctgcctcatcctcctgagtagctaggatcagaggcatgcaccaccacgcccggctaatttgtgtgtgtgtgtgtgtgtgtatttttactagagacgggtttcactatgttggtcaggctggtctcaaactcgtgaccttgtgatctgcctgccttggcctcccaaaatgctgggattataggcgtgagccaccgcacccagcctaattgagtcatttttaagattccactttatctcctttgttggcttattatttataacaccttctggtgttattttagtagttgctttagggtttatagtgtatctctctaatgtctcccagtctaccttccagtggtatcattctatcttacagatattataagaactttatgacagtatactttcatttttcccttcatgcatttgtggtaatgtttcacataattttatttatttacctacattataaatattacaatatgttattgttttacatagacagccggttatctttttaagatagtagtaagaaaaattttttacatttacccacataattaccttttctagtgctatatacctttgtataaatccagatttccatctgctatcattttccttctgcctgaaagacttcctgtgatattatctataatatggctctactggtaacgaattactagcttttgtatgtctgaaaaagtcttcatataaccttcattctagaaagtatgtgattcaaagggccgggcacagtggttcacgcctgtaatccgagcactttgggaggccgaggcgggtggatcacctgaggtcaggagttcaagaccagcctgaccaataaggtgaaaccctgtctttactaaaaatacaaaaattagctgggcatggtggctcatgcctatagtccctgctacttgggaggctgagacaggagaattgcttgaacccaggaggcagaggttgcagtgagccaagatcacgccactgcacaccagcctgggtgacagagcaagactccatccccctgcaaaaaaaaagaaaaagaaaaagaaaaaagtatgtgattctacattggcaattttttttttttttttttttttgagacagagtctcgctctatcacccaggctggagggcggtggtgccatcttggctcactgcacgctccgcctcccaggttcacaccattctcctgccccagcctcccaagtagctgagattacaggcacccaccaccacacccggctaatttttttgtattttttagtagagatggggtttcaccatgttagccaggatggtctcaatctcctgacctcatgatccgcccacctcggcctcccaaagtgctgggattacaggcatgagccaccgagcctggccactttttttctttaaatgcttttaagatgttcctactatcttcttgtttttaattaattaatttattattattattattattattattattattatttttttttttttttttttttttagagacagggtcttgcgctgatgccgaggctggagtgtgctagtgccatcgtagctcactgcagtctcaaacacctggtctcaagcaatcgtcctgcctcagcctcctgaggaactaggactagaggtatatactaccatgcccagccaattttaaaaattttttgtagaggtggagactcgctatgttgaccaggctcctctcgaactcctggcctcaagcaatcctcctacctctgcctcccgaagtgttgggattacagggattacaagtgtgagccactgtgccagtccccactgtcttctggcttgcatcgtttctaaaagaaacttggtgtcatccttatttttgtttctctacatgttatatgtcctctttatctggttgctttaactttatttattaattttagtttaatttttaattgacaaataataattgtatttttatggggcacaatgtgatgttttggtctatgtttacattgtggaatgtgtaaatcaagctagtgaacatatccaccacctcacacacttaccattttttgtgtgtggtgagaacatgtaaaggctgctccttgaggccaggcccaatcccagcactttgggaggccgaggcgagtggatcacttgaggtcaggagttcaagactagcctggccaacatggtgaaaccccgtccctactaaaaacacaaaaatcagccaggcgcggtggtacacgcctatagtcctagctacttgggaggctgaggcaggagaatcacttgaacccaagaggcagaggctgcagtgagccaagatcatgctactgcactccagcctgggcaacagagcaagactccatctcaaaaaaaaaaaaaaaaagtctattccttgagcaattttgaaatacacaatacatcattgttaattatggtcaccatagtgggtagtagatcactaaatcttattcttcctgtctaactaaaacttttttccttttgaccaacatctccccattccctccctcaacctcagcccctgataaccaccattccactctctactgctatgagtttgacctttttagatttcacatatgagatcacatggtatttgtctttctgtgcctggcttcttttacttagcataataccttccagatttacccatgttgttgcaaatggaatttccttcttttttaaggctgaatagtattcgtgtgtgtgtgtgtgtgcgtgtgtgtgtgtgtgtgtatcacattttctttatctcttcgttcattaatgatcatttaggatgattccacatcaggctactgtgtatagtgctgcagtaaacatggaagtgtagacatctcttcagcatactgcttccaatctctttggatataaacccagaagtgggattgctggatcatatgtagtgctatttttgtttttttgaggaacctccatacttattttgcataatgctattctaattcacaatactaccaacagtggacatgggttcttttttctctacatgcttgccaaccacttgttatcttttatctttttatatatctggctgcttctaaatttttttctttcttaccaattctgaaccatttgatggtttcttcctttatgctccttgtgcttgaggttcattgagcatctgggatcagtgcacttattgttttcatcaaattcagaagattaggccattatttcttcaaacttttttgtcgttctctgtctacctttgagagctccaattatacatacattaggccacttgaagttgtcattacagttcactaatgctaagttctttttttaagtcttgtttctgtgtttcattttggacactttctattgctacatcttcaaatttactaattttttcttctgcaatatctaatctgctcctaatcctatccagtgtattttccatattagatattgtagttttcataactagaagcatgatttggttctgttttcacccatgtatctatataacatgtccagtctttcactcagcttcttaaacatttagaatatggtcagaataactttttttgctgttttgttttagagacagggtctcactttgttactcaggctggagcgcagtggcatgatcacagctcactgcagccccaacctcctcgtctcaaggaatcctcccacctcagcctcctatgtagctgggaccacaggtacacaccaccacacctggctaatttttaaattttttgaagagacgggtctcactttgttgcccagactggtctcaaactcctgggttcaaacaatcctccagccttggcctcccaacgtgttgggattacaggcatgagccactgtacccagcccagaataactttttaaaaatgtcttgaggccgaggttgggaaataatctgaggtcgggagttcgagaccagcctgaccaacatggagaaaccccgtctctacaaaaaatacaaaattagccaggcacagtggcacatgcctgtaatcccagctacttgggaggctgaggcaggagaattgcttgaacccgggaggcagaggttgtggtgagccgagatcacaccattggactccagcctgggcaacaagagcgaaactccatctcaaaaaaaaaaaaaaaaaaaactcttagccacaatttctatcatctgtgtcacttctgagtccctttctattcagttatttttctccttgtcatgggtcatatttttctgattcttcatgtgtcctgtaattttcttttctttttttttttggagatggagtcttactctctcacccaggctgtagtgcgatggcacaatcttggctcactgcaacctccacctcctgggttcaagtgattctcctgcctcagcctcccaggtagctgggattacaggtgctcaccaccatgcccagataattttttgtatttttagcagagacggggtttcaccatgatggccaagctggttttgaactcttgacctcaagtgatccgcccacctcggcctcccaaagtgctaggattacaggcatgagccaccgtgcctggccagttgttctcattggatgtcatatgttgggaactttattgggtgatggatatttttgatttcctataaatattcttgaactttgttctgggatgcaattaagttacttggaaaatctttgatcctttcaggtcctgtttctcagcttcattagatgggactatcacagtgtttgttttagagataactttgccccactgctgaggcaaaaccactttgagcttcacctgatgccccatgacttcagtgatcttccactgtgggaggcgagagcaggactatatccagctccatgtgggccccaggcagcgttcactatcatcatttcaggttgctactgaagtatccctttttcaggctctcagctggcagagcaaatacatatatgtatacatactaacctatgtctatacaggaatctatcggtatttctgtctgtggccatctgtagctgtatgaagccaaacatgagtgtgtgctgatgtctccagccctcatctgttaccagatggatcgttctagcctcctccacttgcctacctgtcaattcaccattccttgagttcatggttcattttcagtatacctgcacagtggtatcagaactgttaacccacaccctgtgggaaaaaaactccatcagctagagcacagtgtttacagccagatccttttgcctttagtcttacagattccaatcattccaaattattcggtgcagcgcctttccgcacctgcacccactttttcccctgagattgtttcctacattcgtagcacagttagattgttttgttacattctgcatttcaccctgggatcctccaacctcctaagttatttttgttttatttgcacacattaggttcaatctgaactataaagttctgtgggttttcacaaatgcgtagtgtcatgtatccaccactacattttccttctctctctttcttgctttctcgccttcttgtcttgctctgtcacccaggctggagtgcagtggcacaatctcggctcactacaacctccgtctcctgggttcaagccattctgctgcctcagcttcccgagtagctgggactacaggcacgcaccaccacccctggctaactttttgtatttttacaaaatacaaaagacgatgtttcactatgtgggccaggctggtctcgaactcctgaccttgtgatccacctacctcggcctcccaaagtgttgggattacaggcgtgagccaccacacccggtctctctccttcctttcctttcctctcctttccttttctttctttctctttccctctcctctcttctcctctcctctcctttgatggaggtctcactgtgacacccaggctggagtacagtggcagcataatctcagctcactgtagcctcagcctcccagggctcaggtgatcctcccacctcagcctcccaagtagctgggattacaggtgcacaccgctgagcccagcaaatttttgtattttttgtaaagatagggtttcaccatgttgcccaggctggtctcaaactcctgagctcaagttatctgccagcctcggcctcccaaagtgctgggatgacaggcatgagctaccgtgcccagaccactgttagattttcatatgaatagtttcaccacatcaaaaaaccccatgcttcacctattcaaccctgcctctcccacccccagccagctcagaaatggttctttttaccattgctataattttgccttttccagaacgccatgaatttgaaatcatatagtatgtagccttttcagactgacttctttcatagcaatatgcatttaagagtcatccatgtctttccatggcttgatatctcatttctttttacactgaatgagttcccactgtctgtttgtaccacagtttgtatatctattcacctatctaagggcatcttggttgcttccaatttttggcaattaataaagctggccatgcacagtggctcacacctgtaatcccagcattttgggaggccaaggcgggcagatcacttgaggtcaggagtttgagaccagcctggccaacatggtgaaacgctgtctctactaaaaatacaaaaattagccgggcgtggtaatgggcacctgtaatcccagctacttggaaggctgaggcaggagaatcacttgaacctggaggcagaggttgcagtgagctgagatcgtgccactccactccagcctgggtgacagagtgagactctgtcccaaaaagaaaaagaataaactgctgtatacatgtgtaggttttgtgtggacagaagttttcaaatcagttggacaaatacctaagagtgtgattccatcatacagtaaaactgctttgctttgtcagaaactgccagaatgtcctccaagggggctgtctcatgttgcattcccaccagcaatgaatgggggttcctgttgctccacatcctcaccagatttgatgatgtcagttttgtggattttagtcatcctagtaggtgtgtggtgacaccacattgttgttctcattctcagtgccccgatgacatatcatgctgagcattgtttcatatgcttacttgccatctgtatatcgtccttgctgaagtgactgttcagatgttcagatcttttgcccattttctttctttttttttttttttttccttttgatacggagtcttgctctgtcgccaggctggagtgcagtggcacaatctcagctcactacaacctttgcctcccgggtccaagcgattcccctgcctcagcctcccaagtagctgggactacaggcacgcaccaccatggcaagctaactttttcttttttttttcttttcttttttttttgagatgaagtctcgctctgtcacccaggctggagtgcattggtgcgatcttggctcactgcaagctccgcctcctgggttcacgccattctcctgcctcagcctcccgagtagctgggactacaggcgcccccaccacgcccggctaatttttttgtgtttttagtagagacggagtttcaccgtgttagccaggatggtcttgatgtcctgacctcgtgatccgcttgctccggcctcccaaagtgctgggattacaggcgtgagccaccacgcctagcccccatttttcaattgagttgtttgttttaagacctctttgtatattaccacatgtgtattgaaaatattttctcccagtctgtggcttgtctttaattttcttagcaatgtcttttgcagagcagaaggtttcattagctttcatagattccaacttatattttctctttcatggattgtgcatttggtgttgcccacacagatttttatactgtattctggtgccatttactgagttaacaattgcggaagaactggaagaaaggaagcaaacaaaacgagttctgcgtggcactgtcagtgcgggggcatggggagtcctgcagggtgaggtatgggcggtatggcaaggcgcgggcccatagatgtgcaggtctggagatgtgtgcagcggagatgtgcgggcccgagatgtgcgggtccgatgtgtgggtccggagatgtgcgcgtacccagaggtgcagatcggaaatgtgggggtccggaggaaatgtgcggatcaggagaagtgccagtcccgagatgtgcggatcggagatgtggagggctaggagatgcgtgggtccggagatgcgcagatcaggagatgggcgaatcggagatgcgcgggtccggaaatgtgcagagcggagatgtgtggatcaggagatgttggggggtcaggagatgcgggggtccagagatgtgggggtccggagatgtgcgggtctggagatgtgcagagcagagcaaagatgagctgatcggagatgcccaggtccggggatccacgggtccggagacgcgcgggtccggagatgcgtgggtccagagatgtgcgggtccaaagatgtgcaaatctgaagatgtgtggatgggagatgtgcaggtccggagatgcgcggggcggagatgtgtggatcggagatgctcagatcgaagatgtgggaatgaggagatgtgcggggcgggatgtgtggatgggagacgcgcgggcccggagatatgcggggcggagatgtgcgggtccagggatgtgtgatctgaggtgtgtgggtccggagctcggggtcagctcagcagcagtgagagcgagcatgctggctttgggagcacagcacaatggcagctgtaggagtgcaagagggtgtgacccagaggcagggcccggccccgcatgggtgttctgaggtttatgcctcagcactagaagcctcgtatgcgaaatcacatcctcatagacccggttcagacacaggatagtgatgcctggactattcatccgtctctcctctttttccccagacacgctttcaccagcttcgagcccctcctcggtgacttatcctgtggtccccggcagcgtggacgagtctcccagtggagcattgaacatcgaatgtagaatctgcggggacaaggcctcaggctatcattacggagtccacgcgtgtgaaggctgcaaggtagaggggagctggaacagggcctggtggccgccaccatcaactacttatggtcacttttatagcaaatggcagtcattactgagagattgcagaaagtcccggataagaaactgacttcaggccaggcgcggtatctcatgcctataattccagcactttgggagaccgagatgggtggatcacctgagatcaggagttcgataccagcctggccaacatgatgaaaccctgtctctactaaaaataccaaaaaaaattagccaggcgtggtggtgggcgcctgtaagcccagctactcgagaggcaaagacaggagaattgcttgaacccaggagccagaggttgcagtgagccaagattgcgccactgcactccagcctgggcaacaagagtgagactccatcttaaaaaaaaagaaagaaaaaaagaaaaagaaaaagaaactgacctcagtgatagattagcctctctttatagcacagaacccctgagagcgtaagccctgttgtgaactgcgtatttgaggaatctagcttgtacgccccttatgagaatctaatacttgatgttccaaggtggaacactttcatcctgaaactatccctccccacccccatctgtggaaaaattgtcttccatgaaaccggtccctggtggcaaaaaggttggggattgctgctttagagagtctaggacaaatggttcctctgtgctttgtaaatacttagagaagtgcattctttaaaagaaaataagtcacattggaccgggtgcagtggctcacgcctataatctcagcactttgggaggccgaggcggctggatcacctgaggtcaggagttcaagaccagcctggccaacatggtgaaaccctgtctctactaaaaatacaaaaattagccaggtgtggtggtgggtgcctgtaatcccagctacttgggaggctgaagcaggagaattgcttgaactcaggaggcggaggttgcagtgagctgagatcgagccatttcactccagcctaggcgacaagagtaaaacttcatctcaaaaaaaaaaaaaagagagaaaagaaaataagccacattaagaacatcacttcattcgaatacaagacagagagctgttaccgttgatctctggagcctccctgaaggccaggtggggcaggtgttctcatgctcctgccagggaaattggccatcagagacacagagtatcttgcttagggtcccacagcccccagcagcagggactggaaccagagactggctgctcctgctccccagcagttccttcctgcacatcaggggcttctccacctgattcaagcgacaggaaccccctgtgcatcttcatcctcctgctggctcagcctgccctaaacagatgtgacctgggccaggagtgcatgaaggcaggccctgttgtcctgcatgctgccagctggactggtggcccttccgtgtttgtcagcgtggtgatgaggagagctcctgtagcagcgtccctttagggttgcacagacgtgctcaagtctggcgccttatgtacgtgatatgtgggagatcatcatctgaatgtttggtttgaatcagaaatcccttctcacggtgcacgctgcaggtgttcactaacttggaaaatgccaccgcctttctggcacaatgtaccatcttggaacaccagcattctgccctgagccaggcctggcctcagaggcctgggccacagggagaacctcacagccaggacactgtggcactctgctgtctagaagcctgtctccccacccttcccattctaaccccatgcgttcctcagcctccccactgtgcaagcctaggtaaggacattatgaagacgtcagcctgcctctcacattcccctgcacactgctgtccctctcccgcgggccaagcagacccactgtggcaaaaatatagaagaatgacttaaaagcaaagagaaaaaagaacccaaagcaaaaatgaactccttcgcatgttttctaaccatatacctttgaaaaagctccttataaagtggccttttccttagggccatgattaattattcatttagttttgttttttatggactatttagtaacattgtttcttgctgggtagagtttaagatgcttttacaaagcaagaaaattgtttacaaacagctggcttccttttattataatttttgtctttgagggagttaatatactcttacaaaaattcttagaaagtctttagtcacaaatatggaaatgtcacaatgctggggatagttacattcatatacattgtaacaaggctgagtaactctttggaaaactataattgtgttttcccaagtcagatgagggcattttgaaatgacttcgaatgctgcctcattttattgtttttcacattaaatgtaacgacatttaaagttctgtatttgtcctaatcattccagacttcttagaagaactatttctttctttttttttttttttttttttttttttttgagatggagtctcactctgtcgcgcaggctggagtgcagtggcacaatctcagctcactgcaacctccgcctcctgggttcaagtgattgtcctacctcagcctcctgagtagctgggactacagacttacatcaccatgcccggctaatttttgtatttttagtagagacagggttgcaccatgttggctaggctggtctcgaactcctgacctcaggtgatccacccgcctcagcctcctaaagtgctgggattacaggcatgatcaccatgcctggcctggaataacttttctctaaattttgttcatttaaaaagaaacaataaatgagcaacaaaaaaggtgagtaaagcaagtgcgctggtttctcagtggcccaggtctttaaatccactgtgtattaccctcacagggcttctttcggcgaacgattcgactcaagctggtgtatgacaagtgcgaccgcagctgcaagatccagaaaaagaacagaaacaaatgccagtattgtcgatttcacaagtgcctttctgtcgggatgtcacacaacggtaggtaaggtggccctgcacattttcccagttcgttcctcagttccccttccttgctccaagggaacagatcaagctatggatgaatgtgcttcaacatttcacacccaagtcattttgtaatcagagtggcctaagaaaataaaagtcgcccaggcgcggtggttcacgcctgtaatcccagcactttgggaggctgaggtgggtggatcacctcaggtcaggagtttgagaccagcctggccaatatggtgaaaccccgtctctactaagaatgcaaaaattagctgggtgtggtggcacatgcctgtagtcccagctactcgggaggctgaggcagaagaatcgcttgaacccgggaggcggaggttgcagtgagctgagattgcgccactgcactccagcctgggcgacagagggagattccgtctcacaaaaaaaaaaaaaagaaaaagaaagaaagaaagaaaataaaagtctcccaggtgcggtggttcacacctgtaatcccagcactttggaggccgaggcgggtggatcacttgaggtcaggagtttgagaccagcctggcgaacatggcaaaaccccgtctctaataaaaatacaaaaattagctgggcatggtagtgcacacctgtaatcccagctacttgggaggatgagacaggagaatagcttgaacccgggaggcggaggttgcagtgagctgagatcgcaccactgcactccagcctgggcgacagagtgcgactccgtctcaaaaaaaaagaaaaaaaaagaaaaagtctcaaatagctgagattcagtggtgcattggactcgctgttagaaacttcagtggtaagactttgatacagaatcgaaaaaccaagtggaaggcaccaaaatgacagaatgttcacctcgtccataggaagggtgtaccacctcaaacatctcaccacgttatgaatttccttctagccaatcatttaatagtttcagaacatgctaattgtgatgtgaatgtaagtcgttcataagagttgcatgtctaccttctggaaaaagaagcagttattatataaactcatcccgaagccccgttcacctccttcactcaaaggttgatgatgcacctgatagtggtgtgcaccctactaatgagacgaacgatggtgtcaccttcagcctgcacctgttaacgatggtgtcaccttcagcctgcacctgtttaaacatctacagtgtatggagtttgagtttttcatctctccatagtggaaagccgaatagtaatgaaggatgggtctgaactgcctgtgaattttcattcctggtttaaagtcctgggggagcccctcgtccagccctgtccgcgcagtcatgacctcactgctcatgcctgtgtttccccctccaaaccctagcgattcgttttggacgaatgccaagatctgagaaagcaaaactgaaagcagaaattcttacctgtgaacatgacatagaagattctgaaactgcagatctcaaatctctggccaagagaatctacgaggcctacttgaagaacttcaacatgaacaaggtcaaagcccgggycatcctctcaggaaaggccagtaacaatccagtaggtgtttgcggctgttctgggttctcttggcaacatggaaccagtgtcgtagaggacgattaaggacacatgtgttgaatgttgagaaaattatatttatcccacagttaagcaaaggacagcgaagatggaaacagttcattctgagactctgagctgtagcttaacaacaactcctttcttcttgcttggagccacctcaaagctcttagcaactaagttattatactggctatgtaattaatacacttaaaaaaaaccttaatagcttaccaagtactaagatgatttcttaggagcattttttcttaaatagagataggttcttgctctgttgcccaggctggaatgcagtggtgcaatcatagttcactgcagccttgaactcctgggctcaagcaatcctcctgcctcagcctcccaaggagctgggactacaggtgtgcaccaccacacctggctatgtttgatgttgttgttgttttgttttgtttttgttttttggtagagatgagatgtttcccaggctggtctcaaactcctggcctcaagtgatcttcccacctcggcctcccaaagcactggcattacaggtgtgagtcatggcacccagcattaactggatttaaaaaaaaaaaaactgaccaggcaagatgggtcatgcctgtaatcctggcactctggggaggccaaggtgggcagattgcttgagtccaggagtttgataccagcctggccaacatggagaaaccccaactctactaaagatacaaaaattagctgagcagggtggcacacacctgtaattccagctacttgggtggcttaggcatgagaattgcttcaacccgggaggcagaggttacagcaagctgagatcatgccactgcactccagcctgggtgacagatcgagaccctatctcaaaaaaaaaatagaataataaaataaatccctactttgaggtgtattagtctgctataaagaaatccctgagacctggtaatttataaagaaaagaggtttaattggctcgtggcccacaaggctgtacaggaagcttctgcttctggggaggcctcagggaatttgactcatagcagaaggtgaagtgggagtaggcgtcttgcatggcaggagcaaaaacaagagacacacacttttcacccatcagatcttgtgagaacgctatcactagagtagcaccaagaggatggtgctaaaccattcatgaaggatcacccccatgatccagtccctcccgccaggcctcacctccaccactggggattacagttcaccatgagatttgggtggggacacagagccaaaccatatcataaggctagaaaaggaaaccacttacttcccactcaaaatgtgctcttggtcctttctcctaaaactactccctccctctcagacaaacatgcctacattctttttccgccttcagtgaaaagacagtgacatcttggggcttagaaagggccacttgtaagccaggcgtggtggctcacgcctgtcatcccagcactttgggaggccaagacaggcggatcacgaggtcaggagatcaagaccatcctggctaacatggtgaaacaccatctccactaaaaatacaaaaaattagccgggcgtggtggcgggcgcctgtagtctcagctacttgggaagctgaggcaggagaatggcgtgaacccaggaggcagagcttgcagtgagccgagatcgtgccactgcacttccagcctgggcgacaaagccagctgtgtctgggcgcggtggctcatgtctgtaatcccagcactttgggaggctgaggtgggtggatcacttgaggtcaggagtttgagaccaccctggccaacatggtgaaaccccatctctattaaaaatacaaaaaattagctgggcatggtagcggttgcctgtaatcccagctacttgggaggctgaggcaggagaattgcttgaacctgggagctggaggttgcagtgagctgagatcgcaccactgcactccagcttgggcaacagagtgagactctgtctcaaaaaaaaaaagaaaggaaaagaaaggaccacttgttatagaaagcctgtcttttaaggtagctctggaccttttcagaggcagccaaattgcccctcatggttcgtcccccacatccccgcctgcctggcctaagtcctccttccccctccccaacagttaaataagtctttgtctccattacaaaacaaatctcagagctaccttcaaagaagagccagccctcagttggtgaatgaagatactttgacattttcctatgagcatggtgaaacaggtttaatttgtattaaatagcttgaagcaatccttattgggaattacaaggtggaattttagtcacaggaaaataaagcatttcacaagctacttactttcatgaacaaaccaaacctcttctttactgagtcctttaattcttcagtgaattctccaattaaataggccgagacattttagaagtttccagcagacacccacactaggcagctccagaggcttgtcccaattagaactttcctggattacgagagtgaaagaaaaggtaacttttagcttcgagtctctatcctggatatgattagtacagcccaaaattgggatggctaaaacttttgtttgccagcttatatttctcccttggatttcagaattgaaagcaggctgggcacagtggctcacactgtaatcccagcactttgggaggctgaggcgggaggatcacttgaggcaatccaagagtttgagaccaggcaacacaaggagacctcgtctctacaaaaaatgattttttaaaaaactagctgggcatggtggcatgtgcctgtggtcccaggtacttgggaagctgagatgggaggatggcttgagcccaggagttcaaaaccaacctgggcaacatggcaagaccacatctctacaaaaaataaaaacattatccaggcatggtggcacatgcctatagtccccgcgacttgggaggttgaggaggatgccttgaggccaggagttcaaggctgcagcgagccacgatcgcgccactgcactccagcctaggcgacaaagcgagactctctaaaaaaaattcgaagcagagttaagttgtctttcttcctaacaacctgcccccaccatggggtgcgaatgggactcctggagtcctcctgcacctccccttggagaccaccaagctctaggaaccccatcaccctcagctgagggtcacatgcagcaactagcaggcgggaatctgtttgcattttggccttaaagaaataaataataggccaggcgcggtggctcatgcctgtaatcccagcactttgggaggctgaggcaggtggatcacctgaggtcaggagttggagaccagcctgaccaatatggtgaaaccccgtctctactaaaaatacaaaaattagctaggcatggtcgtgggcacctgtaatcccaactacccaggaggctgaggcaggagaattgcttgaacctggaaggcagaggttgcagtgagccgagatcacaccactgcactccagcctgggtgacagagcgagactccatctcaaaaaaaaaaaaaaaagagggccaggcgtggttgctcatgcttatgcctgtaatcccagcactgtgggaggcagaggagggcggattacctgagctcaggagttcgagaccagcctgggcaacatggtaaaaccccatctctactaaaatacaaaaaattagccgggcatggcagtgtgcgcctgtagtcccatctattcgggaggctgaggcaggagaatggcgtgaacctgggaggtggaggttgcagggagccgagatcacaccggtgcactccagcctgggtgacagagtgagactccatctcaaaaaaaaaaaaaaagaaagaaatgatagatgaatagtttaggattggggttcacaatttggttttctgtagaaaaagagaaccgggcactcttccgagagtcagatgccctcttccacccacacccacaaagccagagcaccgcaggtaccagttttcaaggcaacctccaaccatcatgtgactctttgtgtttgatcacactgtttgctccaagccagggttgcgtcccaccccatgtccttgtctgcgcacgggacgctggaggcacggccccctcctccctgcctagcctgctgacgggctttccagagctggctccttcaggtgcaggataccctctctgcttagtctgggaaaaggccccgttggcaggatgcccaccaccaggccacactgcctgaatctattggcagagctctggttttgtggccaaggtgggtagtggaagaccatagcctgtgtcccttacacatctcagaaagcaaccccatctgtgggcaagaaatctgttagggagaccaagcagcggcctggaaacaccttgatctctgcccagtggcccacatgcggtcgccgtttcatcagtttccagcctgggtgacctcacagccccagccacgccccacagagcctcaggaaggcacactgacctcagggccggcggctgacttcatttctgtttggggatgagaggcggcacagtaaactgtccaggccagtaaactaatggattcatacgaaccgtaatgaacgtgggctgtgtgctggggaaggcaggctcgcctcctccctgcaggggctgctggggtgaaagcaaccctgaaatgttcaaagccttgatggggaagcacgggggatggatagattttaatttcaaagcagccctctggtttgctataagcgggggactgaatttctctttgcagtggccaatgcctttcttctgtcaagatcagctcgtggccttcagatcagatgacgcaaagccccatggctgagctggaacaggctagaatgctgggggggggcctgaaaccggtgggggagttgtgggaggcctagaatcagccaggaggcttgggtcggggttggaaccggccagggtgcacggaggaggctgtgggggcagggggaggccgctgcatggagccgcatagatgccattgcttgaggaaaggtgggctttagctgagggaaggagtgaggggtggatggagaatgtctgtgtccatctggacactgggactgtttgagcccctgagatttcagaaccgtgggccagaaaatggtcagggcccttggtgatggggaagggcgcctctggggaactcactgccccttgatttgagggtaacagggatggaagcagagtcagggggctgagggaggcaataaaaatgggtgcttttcaacagtgtctaaaaacataagatgttgacctgtcaggggttgagaatgtcgtcagaagactttggaggaagcaacagaaaatgagactgaggggcttgggcagagtcagtgccttctgtgtgatgcacgctcatgcacaaatgcacgcacatacccacactcacacatccgtgcacacacgggtacacacacatacacgtgcacccacatgcatgctcacacacatgcacccacagtcacacatccatgcatgcatgtgtacacaaacacacccacacatacacatgcacccacacgtgtacacagatgcacctccacccccatacatgcacatggacacacacatgcacccacacgcacacaagcatccatgctcacatgggtacacactcacacatccatgcatgcacgtgtaaacacacacacccccacacatacacgtgcacccacacatgcacacagacgcacctccacccccacacacgcacacacacacatgcacccacacatggatacacgcacactcacacatgtacccacacctgtgtgtacacacacacatgcatgctcacacacatgcacccaggcacacacaaatccacattcacccatacagtcacacacatgcatacacacacatacaaacacatgcattcacacagatgcatacacacacacacttacaaactacacatgtgcttatacatgctcacatgcatgtatatgcacacacataccctcaccttatgcacacatgtacccacacacgtacccacacatatacaagcatgcacacatatatatatatacacatgctcacacgcatacccacactcacatgtgtgcacatatgctcacacacacgtgcacacacatgctcacacacacacttactgttgctcaggcttagctgctttgggcttaagaagcaaactgcaccttccaaaaaatgagtgtggtgttcagttaaacaaccaaataattctttagcactgaatatgtggactttagaaattcaaactataaggtgataataacgttgtcctgctactttttaatctaacaaacatatcagaactgacactcagttcaaatgaagaaagtaggaattgggcgtgccgtgttattttttcaaagattctcctattgctccaaattgttggggattatcttaaagtctttgaatagcttcagttatggaagattttaccctctgagaatagaactgaattttagacaaaccatgagtccattgtagctagactggcatgcaagttgggattaaacagagtaaaacgtcttgtttaaaaaaataagaaaggccggcttgggcaacatagtgagacctcctctatgaaaagttagctgggcatggtggtgtgcgcctgtggttccagccgctcaggaggccgaggcaggaggatggaggtcaagactgcagtggactgtggttgcgccactgtactccagcctgggtgacacagcaagaccccgtctcaaaaaaagaaaacagaaaaaagaaaaaaaaagttgagcaaggagactaatttgtgacatgcagctgaacatggtttttaagaccagttttgaaagaggaattccaacattattcttaacatttcagaagcctgggcataagggtgacctccagggtgccgtgttataacaggactgctcctttcaacagctatgaccttataccatgtcttggggtgttgcctgccgtgtgacagtccaatattatacctactacttaagttttctttagattaaaaaatgtgcttcatattttatgccatttctacaaatgtatagtaaaacataaccaagagagcttattaaataatttcatccaaagcagttctaccagtgcttcacatttattttttatttatttatttatttttgagactgagtctcactctcttgcccaggctggagtgcagtggcgcaatctcagctcactgcaacctccccctcctgggttcaagcgattctcctgcctcagcctcctaagtagctgggattacaggtgccagccaccacacccgactaatttttgtatttttagtagacacgggcttttgccatgttggccgggctggtctcgaaatcctgacctcaggtcatccacctaccttggcctcccaaagtgctggcattgcgggcatgagctactgcgcctggtccacatttaattttttgcaaaaagatgacagctgctaacagagatgaattctcatgagtgatatcattgagcttcgtaggccacatgagtgtgtgccgggaccagtgtggcagcaagcggggcgttctgctctcggcatggagtgattggggaaaatctaggcagcttcctgcctcacgctgtttaaaacctttataatgtgctttatttcatttatttgaaatgactgcctgtcgtgtcagatatattcatagtcaagcttgagtataaaaggcatattccaaagttaaatataagctgctgcatagatttttttgtaaaatgatctcaccaagaatgtttatccataaagtttagcgaatttgcaagtgtgtttttcaacagcatttctctttagctttaataaacattggtttcttcatggtaccactcattttgaattcagtggtctccagttctccctgctaaatgaggcccactttctaaaaccaaagtgataattttataaaaatgaaatgagatatttgttaccacagaagtcctcatttacgagagtacatccccatagaactagtccacggtgagcctcaggggcatgcaagctgtttaacgatgcccccagcctagaaaggcccaggcttgggtgttcatgctccgctgttgccttcttgaaattcataatcatctttgaacaaggggtcccgcagtgtgtggtggctcacgcctgtaatcccaacactctgggaggctgaagcgggtggatcacctgaggtcgggagtttgagaccagcctgaccaacatggtgaaaccccatctctactaaaaatacagaaattaaccaggcgtggttggtgggtgcctgtaatcccagctactcaggaggctgaggcaggagaatcactcaaacctgggaggtggaggttgcagtgagtcgagatcacgccactgcactccagcctgggcaacagagcgagactccgtctcaaaaaagaaaaaccaaggggtcccacatttgcatttttgctctgggtcctgtaaattacgtagccaggcctgcatttgtcctgggagatgctctaccaaaaaacaataaataacaccaagcattctgtaatcaaacactgtaggaacccctgcttatcctagcctcattctcattctggaagactgcacatttatcatgttaaagactcagctagggaggcccaacttcattcaactcagtgtttcttatttttttaaaacagaactcattttttaaaaaaattattggctgggcgtggtggctcacgcctgtaatcccagcactttgggaggctgaggtgggcggatcacgaggtcaggagatcgagaccatcctggctaacacggtgaaaccccgtctctactaaaaatacaaaaaaaaatgagctgggcatggtggcgggcgcctgtagtcccagctactggggaggctgaggcaggagaatggcatgaacccgggaggcggagcttgctgtgagccaagatcacgccactgcactccagcctgggcaacagagcgagactccatctcaaaaaaaaaaaaaaattatttaacacctttatttctgctgaatgtactttagaaagattgagtgatttgaataaagtgacggtggcctaagagtctattttctggaattgagggaatactgccatcgatccttgaaaaatatttatttagttcctcctagaggccgggcacagtggctcacgcatgtaatcctggcctgcactttggaaggctgaggtggacagattgcctgagctcaggagttcaagaccagcctgggtaacaaggtgaaacccgtctctactaaaatacaaaaaattagctgggcgtggtgatgtgtgcttgtaatcccagctactcgggaggctgaggcaggagaattgcttgaactcaggaggcggaggcagaggttgcaatgagctgggattgcaccactgcactccagcctgggcaacagagcaagactctgtctcaaaaaaaaaaaaaaattatctagcccctcctagaaatgttaattccttaaatctgagcttcagctttctgtgaagcagaattatctccaaactttaacaaacaatggtcagaactgtttttaaggtcttggagagagatcattttcagtctttattaatcggacttgagattatttagaaacttggctctgaatattgtattcagaatgttttcactcatttgtgagtaattttttaaatatcccctttcctcagatgcagaatcagggctttttgtccagcattatgttgcaagtcctggttctgttgaaacattccataccatctgtgtgatggttatcggcacctccaccggtgccctgaagacagttttgtgctgtgagtccagaaacaggaaacacttcaggctgtgtgtcagaagcattgtcagtggttgtgttttgcccactggcagggggcattctttaaatcctgggatgcttctgcgctttgggctccactgttccagcagtgattagaaataacgctgtaggccgggcgcggtggctcacccctgtaatcccagcactttgggaggctgaggtgggcagattacctgaggtcaggagtgcgacaccagcctgaccaacatggtgtaaccccgtctctactaaaaatacaaaattagctgggcgtggtggcgcatgcctgtaatcccagctactcagaaggctgaggcgggagaatcgcttgaacctgggaggccgaggttgcagtgagccgagattgtgccattgcactccagcctgggcaacaagagcaaaactctgtctcaaaaaaaaaagaaataacaccttagcccactgcattattgacctgtgtctgcatgagctgtggaccacattataatcagagagatctctcagatgttgtcactttcctgctctacccgcagatgtaaatttcagccaacagcagtgtttgtgctcattttccccggctctcccacacatgtaatcccttctgagcatgttggcttcaaataatatggccagccacctcttccaccacgagatcttcaggaaatggcaggccactgggtttacatgcagatggcatgggagcacacaaggcacggctgtggggagttggcacttgctccagaatatggagcaccgagtgaaggtttcagtttcctgcactgagagaaacaagggcattccgaggcttttccactttatccctaaagagtttcacaacgcttgtttgccgatttctacatagatgccacctttctgagttgtatgtatttacatgccaaatgtattcattgagcagcgttaaataatggtgttcacccctaaagtgcatatactggtaaaattaagaatgatcgtaattaagcctcttgcaatagtcattagttcagagaatatttaagaatattaaaggtgctttgctaatgtcctcgttagttttgttttgacaaaatcagtacttcagtttcttgtttcttttttttttgagacggagtcttactctcgctctgtcgcccagactggagactggagtgcagtggcacgatcttggctcactgcaacgtccacctcccaggttcaagcgattctcctgcctcagcctcccgagtagctggggttacaggcacacactatgcctggctaattttttttttttttttgagacggagtctcgctctgtcacccaggctggagtgcagtggcgcaatgtcggctcactgcaagctctgcctcctgggttcacgccattctcctgcctcagcctcccgagtagctgggactacaggcgcccgccaccacacccagctaatttttttgtatttttagtagagacggggtttcaccatgctggccaggctggtctcgaactcttgacctcaggtaaaccacccacctcagcctcccaaagttctgggattacaggcgtgagccaccatgcccagcccagtacttcagtttcttagcgatgaaatccacccaatgtcaggcgatgactattattattttactgatttatactgtttgttctctattaatgtcttattttccccaaccgattttgaagttgagtaaggactatgttccgcgggtatcttgagtcctctgaggcactgagcttggtgatttggacgcaggagctgctcattagtgagctgatagctgggagcatagcgcatcccacatcacctgacttaccttggtgtcctcctttgtagccttttgtcatacatgatatggagacactgtgtatggctgagaagacgctggtggccaagctggtggccaatggcatccagaacaaggaggyggaggtccgcatctttcactgctgccagtgcacgtcagtggagaccgtcacggagctcacggaattcgccaaggccatcccaggcttcgcaaacttggayctgaacratcaagtgacattgctaaaatacggagtttatgaggccatattcgccatgctgtcttctgtgatgaacaaagacgggatgctggtagcgtatggaaatgggtttataactcgtgaattcctaaaaagcctaaggaaaccgttctgtgatatcatggaacccaagtttgattttgccatgaagttcaatgcactggaactggatgacagtgatatctccctttttgtggctgctatcatttgctgtggaggtgagtggttgatttaatctgctggtatcatgtcactgacaggctcctgtcttgaaaaatttgacaatgggaaatccagtaccagcctgagctgttccagtggaggggacactcacatggtgggaagacgtctgacccccagtcactgctgagaattcagtgggaattataacaatattgtataatattatagtatatattgttattatctataaatacatatttaatattatgtaaatgtatgacattttaatcataatattagccaggtgtgggggtgcacacctttagtcccagctacttactcagtagactgaggcaaaaggatctcttgagcccaggagttcaggttgcaatgagttatgaatgcaccactgcactctagcctgggcaacagaacaagacctatttctttaaaaaaaaattatatattttgcacaaatatatatatagagaaaaagaggtcggacatgggcctgtaatcccagccctttgggaggctgaggtgggtggatcacttgagcccaggaggttgagaccagcctgggcaacatggcaagaccccgtctctacaaaaaaaaaaatagaaaaaattagtcaagtatggtggcatgtacctgtagtcccagctacttgagaggctgaggtgggaggatcacttaagcccaggagacaaaggttgcagtgagccaaggtcacgccaccacactccagcctgggcgacgaagaatgaccctgtctcaaaaaaaaaaaaaaaaaaaattatacacacacacacacacacacatttcgtttatattatatctaatattataaacagatataatttatatattatgatattcctgtatatattatataatgatgttgtattcatattatagacaatattgtatgaagtgctatacagatgtcagtatagttgctgtcacagttggttatgttgatgaaaagtatatttcctaatgcaaaatataatatcagtcagcagccaagtggcagtgactgcaaggtttgctttgcccgaggaagcagatcccagggaaggccgatctggtcctctctgtggaagctggctctgcagcctccacatttttggctcggtgtcacgttcctttaaatagccccatctcaggtctaggaaggtcatccacctactgcaaactcggctgaccttacccagggttggtggagacagatggggtctcccacactgcctgcagccatactgcgcctgggggattgactcactgtcagcatggagctgactcagccctaccagccgtgcccgttactgtgtggctgggcacaagtcagatgaaggaagtccttgcgctctggcataaagtgtacaaagacaaagcagttatgcataatttgtcctttagtatggtcaggatgtagcattgtgggtaaaatgcagttgcagaactatttatatgtagcatgatcacagttttataaaggaaattataatcctatatcaatcctatgtatatagaaaaatgtccagtgagatatatgttaaacctattatggtgggattaaaattatgaggggggatttctatttttcaaaagattcctccttttttttttttttgagacagagtctccgtctgtcaccctggctggagtgcagtggcacgatctcaggtcactgcaacttccgcctcctgggttcaagtgattcgcctgcctcagcctcctgagtagctgagattacaggaacatgccagcacacctggctaatttttgtatttttagtaaagatggggtttcaccatattggccaggctggtctcaaactcctgaccccgggtgacccacccacctcggcctcccaaagtgctgggattacaggcatgagccactgcacccggcaataattcctctctttagagacttaatagttatagccccagccactctggaggccgaggcaggaggattgcttgagcctaggagttccagtccagcctaagcaacagagcaagaccccatcactaaaacaatacaaaaacaagaattttagaaataaaaacttaataattacatttacaaccaaaaacaatgaagatgtttaaatcctcatcactagcaaccctgttaagaatcatagtaatgactgggtctgtaagggagcaccgcctgctgaacatggctcagggcagtattttctggaccaagaatcaggtctcatgctttgagactgtcccaggatgtctagtgccagctaccccaggcaggtcatctggtgtgaatgttgactcttcctgcaccaagtctcagacctgccccaccctcctccccactctgggtctcctgatcttggctcactgcaatctccgtctcccaggttcaagcgattctcccacctcagcctcccgagtatctgggattacaggcgtgagccaccgtgcctggcctacaaaacctagttctaacacaatcactccttaaatatggtggaacacttgaagcttgatatctagtttggattcaaaagcttcatttcccatattatgcaaaactggtggttgtgatctccagaatgtactgttcctcctactagctctaatttttctccctgacaggtggtcatcaggtaaatcacaagtgaaaaggccgcaccataaggtgtacttagggcactattgccgcctagtagtatgaatatttaggaaagagtactggtcctgtctgtccctacttcacctattgactttggaaaaacctatgtctatcttccagtcaagttgacaatatctaaaggcagctcagtttttttctaagaaaggccacataaaataggcatgtttggttcctgaaactgataagcagttcttgggtgattatcacactcaaacctctctctcttctttcgagactagatcgtcctggccttctaaacgtaggacacattgaaaaaatgcaggagggtattgtacatgtgctcasactccacctgcagagcaaccacccggacgatatctttctcttcccaaaacttcttcaaaaaatggcagacctccggcagctggtgacggagcatgcgcagctggtgcagatcatcaagaagacggagtcggatgctgcgctgcacccgctactgcaggagatctacagggacatgtactgagttccttcagatcagccacaccttttccaggagttctgaagctgacagcactacaaaggagacgggggagcagcacgattttgcacaaatatccaccactttaaccttagagcttggacagtctgagctgtaggtaaccggcatattattccatatctttgttttaaccagtacttctaagagcatagaactcaaatgctgggggtaggtggctaatctcaggactgggaagattacggcgaattatgctcaatggtctgattttaactcacccgatgttaatcaatgcacattgctttagatcacattcgtgatttaccatttaattaactggtaacctcaaaattcgtggcctgtcttcccattcaccccgcttttgactattgtgctcctttataattctgaaaactaatcagcactttttaacaatgtttataatcctataagtctagatgtatccaaaggtgaagtatgtaaaaagcagcaaaatatttatttcaaagacttcacttctgtttcctgaatctaaagaaagacaacatgctgctttttaatcataggatggagaattttaaagaactgtttgggccaggcacagtcgctcatacttgtaatcccagcactttgggaggccgaggcgggtggatcacaaggtcagcagatcgagaccatcctggccaacatggtgaaaccctgtctctactaaaaatacaaaaattagccgggtgtggtggcacatgcctgtaatcccagctactcgggaagctgaggcaggagaattgcttgaaccagggagttggaggttgcagtgagctaagactgcaccactgcactccagcctggtgacagaacgagactctgtcttaaaaacaaacaaacaaaaaaaaaatctgttagataagctatcaaaatgcagctgttgttttgtttttggctcactgttttcgtggttgtaactaatatgtggaaaggcccatttccaggtttgcgtagaagagcccagaaaacagagtctcaagacccccgctctggactgtcataagctagcacccgtggtaagcgggacgagacaagctcccgaagcccgccagcttcctgctccactcagctccgtccagtcaacctgaacccacccagtccagctgtctgtgggaatggtggtgttcttagggacagactgacaccttacttgtcagtgttcctccgggccccatttggcagctcccgtatcttttgttatgttgcttttaaagatatgatgttttattgttttaactcttggtgacagtagatgctctctggagcgcagacgaggcacatgtgtcttcatagcctgggctgggtgggagccagtcaccctgcggatcgagagagggggtagagtcttcttcaaatggcagttttacttcaaatggcagatttcacaagagttggttattttttacaatggtttaggttgttaagtctcctttgtatgtaaggtagttttttcaacatctaaaatttttgttttagccttcaaaaccaacttaccaacctcagtccagctgggaaggcagcgttgattatggtagtttgtcaagaatatatggacctggaaacactttctctctctgtccacctggtagataaattgtcctgttgagaatttttagatctggactggaactgccaggaccaccgcctccagggagtcgctgggcacctggaggtatcgtcgatgcctctcccccatctttagaaaatttggctcttctgaggtcattattattttaagaatgattaggattgataagggtcccatgaccagcattatgaaaatgcgagagtgggaaggacacagtgtgagacttccactagaaaaaagtgaaagttagggttaggacatccttttttaaaaattacaaatttagtccgttttggtttttgtaatcaggctaggcacagtggctcacacatggaatcccagcactttgggaggccgaggtgggaggatcacttgagcccaggagttcgagaccagcctaggcaacatagcaagaccctgtctgtacacaaaatttaaaaattagttcatcggggtggcacacatcagtagtcccagctactctgcaggctgaggtgggaggattgcttgaacccaggaggtcgaggctgcagtgagctgtgatctcaccactgcattccagcctgggtgacagagttagattccaccctctcccaccccggcaaaaaaaaaaaaaaaagatgcaatcaaaggggctgttggccagcaatggcagcagcagcggcgggcagtctgcccaagtgtcttaggaaccaaaagcaaataaaagtgtttccatatatgccaccagccaagtggccatcctaattcagaaagaagctagcctttgagtgtctgtcatggtgcatccgtttcagtattatttcctaaaatgagaagcccctgtgtcaacaagatccaggggctggagcccaatgccaagcctgtgttgtccccagcgaccctgcagctgctcgctctgatgtaccctgtgccattcaaggagatgtggtccaggaaagtgagcctcatggttttcagagaagtcattgttctgtttacattttcataaaacctgtttaaaatagctccccgtctcaggctttcagcagtaacagtgagctgactggcaagttcgatgttagctcccgggacactcagcagcgatggtgagcattttggtttccttaaggcccagcaagacttccagggacatctctggtgaagccagaatggagacacccgtgacctcaggctgaaagtcactcgacattggtctcttgtgttgatagggaaggaaatcaggcattcctatttctttaaataacaaaaccactaattgccactcaatgctggaatattttgggtcacctaatcatagatttctcagggcatcaatactcaaatataggctgattatgccccagttcaaatgggaactattaacagagtgcatttcttgcttgctgggtttcaacagacatcagccaaaagaacaaaagagatgtcaggacagattccaggagtgtcggagcacatgtgtggcacccgctccctctggcagcgaatgtaggaagtcgccaaatttacccactcttcaacaagtcattgtttaaacacggtttttcattttctcaacttttaatagcaaaaagtgccaaagtcctcagagacctaacagccttggtctaccgtgctgaccagggtgaaggcacggcgagggactcctcccagacgtgcctcttgtgtgccagctggctgtggctcgggagcagacgcaggcctctccattgtccaggggagcctggcggcgcatccctcctctcccacctcctggcacttccagctgggtgtcccacatgttggattccgtccccaccacacttccagagaccggagaactgtgcagggcctaaggccgtttggatgaattgtcaaaacaagatgcttccagttacagcggcaggagcgggactgggagcacgggctgacggctgctggtgcctttcttcccacctcgcttgcctgtttccgcttgacccttcctccagctccgatgagaagagtataaagcatcttcctaacgggtgtgtttgctatacgaacataatggacgtgaagtggggcagaaacccagaactcagcattcaaggatgcccaggagagctgtccctgttttaaagagctgtgttttgttttgtttcgcatttagagagcagacaaggcacccttctgctgcgctgatacgtttcttacactgggccattttagacccccagggaaacagccttcctggagcgttgtctggaggttccagggacagggcagcctcccagagccgagcaagagctcaaggtacaaatgagagatttgctataccgtgagaagtcaacaacttagccaccacttccccgcaatggaccatgtaacaaatacctcagcaggccctgcaaaaggccatgctagagctgaggcgcacagcctgtggcctctgtagttagggcaggtgggatggagactccttgagtgcacacacctgagcctgcccacacacaggggagcagcatctcgtatgacgtctggaaggaacttcggttgtgtaaagggagccttgaagatacgtgcaaaaggtgctaccccaatttggtgaaactgacattgggcacgtcttgggcttaggagaagcggccgatggtcccggcctgcagtgacaaacccccctccccgcaccgcccccagcaccccctctcctcttcacctcttcctgctggccacgaggaagccacttcctcagagagaccctaccagatgcggatggaaacagatgcaccaaagcaagccctgatgaaaccgcgacttcctaaggtctgtctcctctgaacttgcacctgggcctctctgtgtttggttccaagcacttcccacctcaaactcccattttcaaaccactgtatctctgcgcacatctgctacttaccagccgcatacatgatggagggttttttggtcctgatccagtggccacacctgtctttgaaatgtctcactgaactccagttttaaaatagattcattgcttcaacacagcaagcccaatgcacccagctaagactggcttgaccgacagcctggcctttggtggggggcttcctggggcctggggaaagctggccaccttcaacagctggtacctcttcaacagtgtggcctttcaaaatgcagatgccaccaggagaacatgcccacagctcaccacctatggatgccatggctctgggcagctttcaaagcaggttcctgtggtctcctcagctgtttgagggggtaacagcaaatcagcctccattttaaaatgaaaacaccagcctccagatgtagggcctgctgggtgttgctagccgctggtccccaggcacggtgcactttctccacctcctgcagcctccctgttgtttctagactcttgcacctggtgagtgcaaggataggtgacccaggggcctgcagccttgtcctcagctcccatctcctggactgccagcctcaccctctgcagttagcatggttggcctgatgcagggatcccgagggattactttttagaccttctttcacattcagaaaagtagtatagattcaggagaggcaagaaaattatgctgtccatagaagtcacccatgaagactgatgccaccacctgaaggctcatgattgttaaaaatgtccacgggaacctctcgtccacaggaggtttgtctcaacacttcccatttttacggcattggcattgccaagcatggggaagtatctgctcttctcatgttaaaagtggcccagcttttcttaactcagtccaagctgacttgtttagctgcactggaatttcttaccaaccaaatatttgcatcgagcaaagggggctgtgtgcacctccctaatggcagcgatgatggctgctgtcattcaagcccatcttcagacgtcacagtctggaagtgaaatgtccacaaacatctgtggcagaaaaggctatacggaccacccagttgtgctgcagctttacagagcaaggaagggttgtggcaaataaatgattaacctgcctcgactgtgctgagggcaacaaaggccatctcaccaaaggattattcgatgccattaaatcatcccgtgaccttcctgcttccgagtccatggcctttgcccagggcatgtactcccctgagaggccttctgcctagaaagatctatgactgggttccaaagttgaggcctaggtttttgctgggatttagatattttcaggcaccattttgacagcattcaggaaaacggttattgaccccatagactagggtaagaataaaggcaataaatttggtctgactcagaatataggagatccatatatttctctggaaaccacagtgtacactaaaatgtgaaattgaaggttttgttaaaaagaaaaagataatgagcttcatgctttgtttaattacataatgatttccattacgctatttctgtgaaatgcagcaggttcttaaacgttatttcagtggcatgggctggaagcttatcacaaaaagccatgtgtgtggccttatcagaacagaaagagacaggctggtgcccaaggctgctgcctgctccaccttttgccagctctggacatctgaggacgtcccggcagatctggaatggggccctcaactgaccatttgcttctcagaatttcagtttgagacatgagaggtataatcagttacttttctccccccagagaaacccttttgtgaggggagaggagctatggtatgtggttcagctgaaacacatacaactgcatccttttggagtcctttgccaacaaaaacagaccaacagaccagatggtgtccatgttcaatatcatgtcttgatggacgcagctgatgacctcaaatacttgagtggtctcatggctgttagatggattatttgaaaaaaaaaaaaaaaaaagagagaaaaaataattgatttttacatcagagatagcaaactaagacctggggaggggggtcagcttttattttattttattttttttaagtttgctagttgggtcaaatgtgaggaggagggagtctacctgccacctcttctcttgcccctcttctgcccacacatccagcatccaaaatccattcatttaatgaattgataaagtgccgtgcaaactggtgcacaaacaggcccccagtccacgcagcctggctcctaggaaaagtggtgaccgggcgtgggggggcatgccgcagccctgggacacagtcgggcaccttccccggacccccaggccttggctgtgcctcaagtcagagagggtcagccttcaggccccggagacgagtgactggccgatcatttcacaataaaatcactcacttttggcaacttcactttttttaaggcacagtcagttccttttctcatgtacctcacaaaagatgaagaccatgtagtactctttttggtaaagttacagtgttcatgttaaatatcacttttttctacattgtgtggtaaaaagaactacgttaatagctatatcttaaatactgtgatttgactttttgaaaaatatcctaatacaaatattttactaacttacaatcactcatttaataagaaacatttggattcttttgaaatcagtgttaattgactcatattcttaaaagcctggctcttgaccctattggaaacacaaaggaagctgaaatcaaacatctaaaatacactgcgtacacgtgtgcgtgcacacacacacacacacacacacacacacagctcttcatttctcctgagccatgcagaatttactttcaatgtggaaatctgttccctttaccacactgtatatgcacagagcacaagagaggctatctctagtcacttccaccagcgaggccttagactccgtattagaggccaccgatttcatacaacagtgtttcgctaaagacccttcactattcttgtttagtaaatagctgtctgctcttcagggaactgttacctatgggttattaccaaagaacgctggcaattggaaatgtcctgatggaaattctttgcacgtgccggttctctggcatcctccaggtggcccaacccaaagcagaaagcagaaaccacagaccccgtgagtctccccataccttgtttccaataacttggcaaaacttcttggtgcatattggttacaccctctgggattcataatgccattaggctaaaaccctaagagagagggttgacagaaacacacgcgagaatgaggcagatcccagagcaaggactgggcccagactctccacatgtgctctactagtgagtgccttatactctcagtattttggggcttacagcttcttatttgtgctaaaaaggtgcagttccaaagtaggaactgccacacaggccccagcatcctctctccaacttcatacctctctcctggtggggggagcgggcatccaggacctccggaatcaaggatgtgcagagaagagcgaaagtaatttttctagtcacatgaactgattggttccaggcaattagaaaatggctataaaataaccttaattttaaaaaaaaatcttgggtcttcgttttcctattaggagactgaactgaccacatgtattgatttatatcctgaatatatgggaacttctgtgtttgggatgtcctactgtaagactgatgaatgtacagagttaatttcagggtacagttttgccttaatggttttaaaaaataaactattttttaaaattttttggtgagttttgagtgatgctgcgagatggactgtttggtctaaaagaaccaagtccccagctgtggggtccgccctccgccagaacagtccctgcgcccctcatggaggctgtctccacctgcacagtcccttttgtgtccttcaacatacagaatggatcaaggacaatctggctccggaatacgacatcctttcgtctgaagtcacatacaacgggaacttttaaagcagtgagcatcttaaagtccatcagtgcacaagtcagtaaaaactctttattcattccttcatgtgacagttggccttgagtagttacaaagacagagcagttcctgcctctcagaattctaagcagacattccagagctcacagatcagtgtcccaccagctgctaccctggaagcttcagggagatggggagcctggagtaggggggtgctgcaggaacccccggcaggcagtggggccaggcttcacaggcacccagggctgagtgcgagatggcagggggcaagcaggcaagcagtgggatggtcttgaggtgtggggtgagggcaggaagcccacggtattcctgacttggagtcgagggagacagaacggaggggcccgagctgaagacagagtctgagggaactggagaggctagaagccagcactcagcttcccaggagaccccaactattgccgggccaagtgtccagggagaacgtcgggtatccaaatgtttcagtggaagattccttttccaaacatgagctggttctttgggtttttaattataaacataatacatggtcaaggttaaaaacattttccaagcaccacataaggatatgaaataaaaattctatccccctctagaccttccagactgggtccagaaggagctgctgtcagtgattttccttacagagagaccattgcagccacctacagtgcaccatggccccgacacagtgtggcatctccccccacacgcacctgcacgcagcctcttaggagctgccctgctgaaggaggtcatccctctcagtcacatctgagcagcactcactcaggcctgggccctgaggggcatgtggggggacctggccgggttcctggctgtgtcttgtctggcatctgcctttggactaccctcttgctacagctccctgggcctgtggctgctccaagctggactccagctcccctgggctgcagtgctgctcccagtggtccagaaacaaggccaggcatggcggctcccagctcagcagcatccctggcgcaggctggcccaggagctctgtgcagggtgccgcccgatgacctagccccagttgcactcacgtccgagaaccgggctggctgggggtcctctttgatggagctttccttttctccaagtcttgccgaatttcctgaggaaaagcattgatgttctcccggacacaagggaggcagaatctcttggagtagaataaactgcattccttaaagaagtggacaaggatggaggcttgagtatcctgggaacatggtgcagctcctcccaggggccagcccttcccacacttggggggctgggctggggccccagcatgcacagtgtgggcaggtggccagagctagaccagcaagagggagagccaggcctgagggcctggcagggtgtccaggagaaaagcaatgcaggcctgggctaccctgggtgggaacagctgggatgggacagaaagactctggggcctgctctgctccatcacggggcagtgacaacaatggaaagctggagcacaggccatgccccgaaaatcccagagtcctcccgtcccagctatcgcagcacgaacagtggccacctggtgctgggagcggcaggggacaggaaggacagccccacagaccaaagacgccatggacgagccagctagtctgggcaggaggccagaaccgtgcagccatgtccgctctcctctctcttacacactagaactcccgagattcatcacggtgcacagcacgtggctaaggatgacattccccagccccctctgtaactgggcatcgccatgtgactcaggctggttaggggcgtcagcaactgtgtgtgccacctcctggtcatgccctttgaaggcatggactgtgctctcacgtcccttcccgctgcctgggatgcaaatatgatggtgggagctccacagccacaagggacccaaatagagactctgctgaagacaacaggggtgcccttccagccaaggccaccagttctggactcttacataggagaaagtaagtaaataactatttgctttacttaggccactcttatttttggtgtctgtctgttcttaaagcttagtgtgctcgcaaacataaggctgccctcctgcagttcttagaaaagacccttcgccatggagatccgctcactaacaacctactgtttaaacaaagaaagctgcagacttgtggtacctgcgtgcgccccccacccccgccaccccgctgctcacgaagagcagcccaaggccaaggtgttgcattcaagacctccacctccccacctggcccacccacctttgcagtcttcctttcccagcttctcctggaccaccagctcctccatgagcctcagatactcccatgccacactgcggaggctcctcccaccctggcccatctcgtccccctggctattgatacagctcccctttgtgtgtgtcaacctgtggcagatgctcagggacttctcagactctggggtcactctgagtacactgggcacgctgtgaaagccatgctgctccacttccatccgtccttgggtttacagctacccaggtgaacctggcccacccagcctctctgaagctcagttccctcatctataaaatgggtggaatataatactgctttacctgactaaggggcaggactgaatgaagcctcagcagtaaaacaggtcccaagcacaggacctggcacacaacaagtgctcatgaaaggacagtcccttctcctggagatccctccaagaacatctagaaagtaagtcaccatcctggtgagagggcgcacaaggactgtctgcactgttggtgggaaggggaggaaccctgaggggccactggggtgggtgcagagccaccaattacctatttccattcctacaccaggctgagaccctctaactgaccctagcttgccctctcacctctccccttcatcagctgaggggccccatctataatggggtgaacccgctgctccctctctcccagccctccctacccagggagagctccaggataaccacagcaaggacaggtggccccagcccacgctgcttacccaccgggcccacacacaccagcctgctgcacaaactgcagcacgagccgaggaccaggaatctgtccttgtcggaggtgaagggatccttcatgacatagctttcctccaggaggctgcaggagagtgaatgcagcgtcagaacagcccaaatctctgccgtcggaaaatcagccatgacctatggccaggcagcagcagcctgcacctctgcagggcctgcatacctgcgtgggctcatcagggcaggggactaggccacccgtgctgcagggcagtgacagaaaggatcagcaattgggggccacactggcactgaaattgaacctgctctcacctctgtaacagccatggtagagagaaccccttgttctcagccaaaagcagggctgctgcaacatgggcccccagaccctgactgattcaccacagtgggggcacaaggctgccacctgaataccccaggcctccctacaccccaagccacaccctcacagcccctggaagccagctggcctcatgctacacatcagaatgagaccccagggtgacatgatgacactcaggctcctggtcagcagagggacgacagccagaaggaaaaagccatgcccctgccactgcatcccatgtgagtaacacaccggccagctttccaacaaagtggcatttgatgaaggacaagctggaggtcaagagataagccccctgacccactggctctaatccaggtctgcaggggagacaggcctcccattaagaaagtgtcatggcaagggtgcagccccaaagggggaggccaccacaggtcccccagctgctgagctgctcccccacagctagagcaggtattggactctgggctaggtgtggaggaaggttctggaagagtcagacctcaagccttgcccaggcctccagagattacaccagaagcattcagagcctggccagggtggggtctaaagctccacacctgagacagttgtcagacctaggcacaccacccaccaaccagcactgggcacaggcctccccggcactggcccaaaaggcatgcactcacaccatcgactgggtgttggggggcttctgtcccacatagctgtacggagctgtcaaggtacagagttcacactcaaacactcccagaggacggcactctacatgggacgccatctgcaagatcaagagatggggtgtccctgggtcacaggctttctcaggaatcctgcccctttcccagctatgaagaccctaaccatatagtcctagcacagtttcttgcctccctaccgtacccttttctctatccccagctgaaacaggttttcaccggtgcccagagtgacaggatttgctgtccatattccaatggtttaaggtttttgtactgctagagagaaggatccagacaggatcaagccagagcacccatctcttcttccaggtctgcaccacggtaaaagcgttccggtctcctgctctgccccattctttaccacaagcccccagtgaacgctggtggagaagggcccagagtgggtgcaaaatctcctgtttctgctgctcctagaagctctatgccctcacattagtccacacgtggtgtggacattagaactgttttcttggccaggcgcagtggctcatgcctgtaatcccagcactttgggaggccaaggcaggcggatcacgaggtcaggagatcgagaccatctagctaacatggtgaaaccccgcctctactaaaaatacaaaaaattagccaggcgtggtggcgggcgcctatagtctcagctacatgggagtctgaggcaggagaatggcgtgaacccaggagacggagcttgcagtgagccgagatcgcgtcactgcactccagcctgggagacagcgagactccatctcaaaaaacaaacaaacaaaaaaactgttttcttatctgcttgtatggaacccccttctcatgttcatggttgggtcttggaggcctctctttccttagatttctggctaaatggctgccatctgatgggttcatcaaaagttacaattttgctgattacccagctccttctttgactatggaatcaagtacgacattatcagctggcccctagctcttggctgtgacctcccctcgcctgaactgagcgtggtgtataaaggcgtatcaaatgcgatggctttccccactgggtgtccccagatgcaactccaggaagcatgggttaccagtgactttaaaagttaagtctggcccggtgtggttgctcatacctgtaatcccagcactctggtagccagaggcgggcggattacctgaggtcaggagttcaagaccaacctagccaacatggcgaaaccccatctctactaaaagtacaaaaattagccgagcatagtggcgggtgcctgtaatcccagctacttaggaagctgaggcaggggaatcacttgaacctaggaggcggagattgcagtgagccgagattgtgccactgtactccagcctgggcgacagggcgagactccttctcaaaaaaataaaaattaaaattaaataaaataaaatattagccgggcgtgggggcgggcgcctctaatctcatctactcaggagactgaggtaggagaatcgcttgagcggggggttggggagcgggggctgggggggcggaggttgcggtgagccgagatcgcgccactgcattccagcctgggcgacagagcgagactctgtctcaaaacaaaagttacgatcgatcgcgggtgtgcgtgaacctgatcccgcttctcttctgcacgccggggcgtcctaggcccctgccgaggctcctgggggcggtgcctgtgaacagggacggctgagagggtccggggatgctggtggtccccgcgagcctccgcctgccctctctcgccccagcgctgcccgaggtcaggggggacagtcccgtgaggcccacaccccgcttctcggtcccgtccccaaagctgagagcggctgcgccgactcctggcgcttggaacccagcaggccacgccggcgcggaactcagtcccggcgagcgggaggcaccctgaggcagcgcagggaagggccacagcacgctcgccccagtcactctgccacctcgaactcaccgccgccctcgtcccacggctccgcgtccaaggcgcctgagcggccccgccccaccgcggcccggccaaccccggtgcagccgcagacgcaggcgcggagcgggaggcgggacctcgcggtggttttccgcctctgccttgcttttccggtcgccgcggtcctctgcgcgggatgtggctctgagcttcggggtagtccgcgaggctgggttccgtgctgaggggcgtcgcgtctggccgtgcgctgcgggagcgcgcgggcctggcccgctgtcctggccgctgcgaggcgctcagcttctttctgagtgttccgccgggttcgacagggtcgaggtcgccggcgcgaggctcccggatagctgaggggcgcgagggcgcctccagtctcccacctgccctcccgcgccgcggactaggctgccagtctaccccaccgggacgtctgcccccgcatgcaggagggggtgtgtcggcccaacggggtctcaggacctgtccctgctcctccaaagcccacgtcgcctgcggcgcccctcgggtctgcgccgcgttctcctttagcctcccgggcctctccgctgcccctcgcccccaggcaccagccacacctgcttccttctgttcccgcccgtggcctctgacacgccgtgccctggacccgaaatgcccgccccactcctggagcctccggtgtgtgagaggcacctttccttggggtcaggggcccggactctgaagccggcctggctttgcctgtgggccccgatcctcccagctcgagtccctcgccccagccgccgcgggccttcacctgtgctttggggacgacaggatggccaccctccttcccccaaagatttgagggttttgctccacgcgggagaaggtctgcctgtgccaccagccggcagcctttcacatgctgttgcgttacggggccctctgtgacctttccctaggccgaggggtgcttcgtctctgttcctgagagaccagaatttattcttggtttgtcaagccacatctgtaggaatgttgcccagttgcagcaggctggggtctgggacttgctcctttggcgaattgagaagtaaggatatagaaggcattttagcactaatggaaaccagaaccactcttagacctgggcaagaggggccccagaaattagcaagcgattagcaagcagctggatttcgatgacctccactgtacttagagcaacggcattcgattcgttggatcatttcaaagatgacatactgggttgtttgtttgtttctggcggtgtctaaagatggaacatcttgaagcattttgaatttgacctgtggctaccctcagatacaatgatggtaaccccatgagtgctgttaaagcaatttgacttattgtagacaggttaacacatgagatgcgttaaaagagttaagtgaaataataggcagcattcaaagagcaaatcgtggtctttggcagaaaatagagttaattttgtatttgagctgtccagttattacaattagaaaacgttttaaagaatccaaaaggatttaaaatattgcataaagttttaggagaaaatggtttccaaaaagcaactgcatatgaaataagcactaaaatgacattaaagtacaatgtaagaaaatcggaactagaagcatttgctggagtctgaagaaaaagatttatggatgaatgattctgaaactaacttctatagagacaattttccggcgatggtagatcaaggtgtagattgtactgaagagagagattcaaacaaagtgagtaatctaccgcagttttcagttttcttggtaataccccaacaacgtaaaagaaaaacgtaagaaatcctgtatggatctaaatgtaagaaacgatgataatggtgaaataataacattattttatataatgaaaatattgtgtaatattttctgcaatcataatttttcatatgcaccctttctacactgtttaaacatgatatcaacagtcatgattcagttctacatataacgctttaagaatttattcataattcgttaccactcagcagagtcaagtttctcccaattgaaataactaaaaactatctgaaagtatactgactcaggaaagtttgtctaatttggcactactgtcaaaagaatgcaattgcgtgaaaatcttcattagtcattcttctgaaatgaagacaagagaaacatttatagaagaaataacataatttatggattatgtgattatgtatgtctttattacccatccaagcattagtggcccatcaacataatacctagagatacacaataaaaattagctatctttaatattttgccaattttcagctatattaaaaccatagttttacacataattttgaattctgttttatgaaggtgtatttgtccaggtaagtggatataaatattttatgtaacagttcgtaagcttttaaatatttagacatacagagatttgtagtcctgccctaggccccacagatgctaggggtgggcttgctgggaaaaatgcatatatgttgtggcaatacttataaaatgttgtatggatgtaatcttaaaagattatcacaatatcagatattgtacattgtgtatttaggtgaagactgtgaaagaaaatctgaaaatcagaaggaattgaatgcagatgaaaatctagagctaaaatattgctaatagcagcaaaagggggtactgtggaagaataacttcttatgtaccccacttcactgaatatcctattcgccccgttctccatttctgccagtaacatggaagtctaaaggcttgcattttgtttctagtgctaactaatgaaattccagaagcaggccttaaaaatgcaaagtcttcataattaacaataagtgttgagatagttgaagggcatatatccccttctctgtacctacagcaattgaactttctctgcatataaaacttgtttttattaaatattaaatactactcttgagtatttaataaggggctaaattgagaacaaaaccaggaatatccatcttcttgttctgtgcaacagagacagtgggagggtattcgtgactatagccaattattctggaatagtcctaccctatccctggaagtataatgagaacaagagtcttaaaagactcctttaggctgggcgcggtggctcatgcctgtaatcccagcactttgggaggccgaggcgggtggatcacttgaggccaggagtttgagaccagtctggccaacatggtgaaaccccatctctactaaaaaaatagaaaaattagctgggcgttggtgggagtgtgcctgtagtcctacctactcgggaggctgaggcaccagaattgcttagtcccacctacctgggaggctgaggcaccagagttgcttgaaccccgtgggcagaggttgcagtgagctcagatcttccagcctgggcaacagagcaatactccatctcaaa